SEQUENCE LISTING (102 sequences)

SEQ ID NO: 1 - DNA, 34 bp, Artificial Sequence
  Synthetic DNA
ataacttcgtatagcatacattatacgaagttat

SEQ ID NO: 2 - DNA, 48 bp, Artificial Sequence
  Synthetic DNA
gaagttcctattccgaagttcctattcttcaaatagtataggaacttc

SEQ ID NO: 3 - DNA, 48 bp, Artificial Sequence
  Synthetic DNA
gaagttcctattccgaagttcctattctctagaaagtataggaacttc

SEQ ID NO: 4 - DNA, 48 bp, Artificial Sequence
  Synthetic DNA
acgcccccaactgagagaactcaaaggttaccccagttggggcactac

SEQ ID NO: 5 - DNA, 772 bp, Drosophila melanogaster
taacattatacctaaacccatggtcaagagtaaacatttctgcctttgaagttgagaacacaattaagcatcccctggttaaacctgacattcatacttgttaatagcgccataaacatagcaccaatttcgaagaaatcagttaaaagcaattagcaattagcaattagcaataactctgctgacttcaaaacgagaagagttgcaagtatttgtaaggcacagtttatagaccaccgacggctcattagggctcgtcatgtaactaagcgcggtgaaacccaattgaacatatagtggaattattattatcaatggggaagatttaaccctcaggtagcaaagtaatttaattgcaaatagagagtcctaagactaaataatatatttaaaaatctggccctttgaccttgcttgtcaggtgcatttgggttcaatcgtaagttgcttctatataaacactttccccatccccgcaataatgaagaataccgcagaataaagagagatttgcaacaaaaaataaaggcattgcgaaaactttttatgggggatcattacactcgggcctacggttacaattcccagccacttaagcgacaagtttggccaacaatccatctaatagctaatagcgcaatcactggtaatcgcaagagtatataggcaatagaacccatggatttgaccaaaggtaaccgagacaatggagaagcaagaggatttcaaactgaacacccacagtgctgtgtactaccactggcgcgtttgg

SEQ ID NO: 6 - DNA, 2362 bp, Artificial Sequence
  Synthetic DNA encoding fusion of tet activator protein optimized for insect expression
atggtaattttaaaagcatatttttttctttgaaattcataagttatcaattatcgatggaaatgtattctatggagaacgttttacccgatgaatgggtgcaaaaattattttaccttcaaatctacaatcaacacacgctaacttttgtgacttgatcaactctcacctggaaaagcaaccaactacaatcaacattctatgggataatcgacaagtgagtaaaattatagccggacctcttagtacagtgtatttaaaaggggaataatattctatcaataggaataaaaataaggtcagcagccatgacttttccatcattttgaatataccttatttgtttcgggattaattgggggtcggaaatcctcttgaattcagaaacgggaaccggaggaaggtgccggtctttcagaaagctgtgaaaaataccaacatttctgctgccaagagctcaataagaagtttcaaaaattgtcttggatgttgcagctgtggctgctaagtaataagacatctattagtatctagatttgttagaccatttaacatagtgttttaaacgatggggttaatagatgagggttaagaagctagttatattactgttgctgtaacgccttcaattgtcggttacagagcaaacattattgaatgttaatgtaaagagtttatttgttttctagtaaacatatagcgattggttagtaatcactaatagaaatttttcataagtatcaaaaaagtaaacctctttttcagtctatgtaataagtaaaccaaggaaagggaaaatatctacaatcaacaagccattgttgcagcaacaaagcaactgaaactacaatcaacattcaataaacttgggtaatttggaatttaattctctgggacacctgtggattacaacaatcaactcgaaacttattatacaatgtaaataaaaattgatatgcatacatgaagatcaagtgaaattccatttagaatcaatttttttcgaatattaagtttcttgctttaatttatctgaaagtaaatagacattccaaattcaagttaacaaattaataatgaattgactagtgatttttaagagaaaaagataagatttaaaaaaggaaagcctttcttgataaatttttgaaccactttatgccgtttcaatcataaaaacttttaagaacacatgactggtaaaattaatttaaaacaaatttaaattttcaacgtaacattcaacaaaaatggtgaaaactatcacggaaattgttaatattaatatgtcccaaaaatagcctttgtatgtatatgatactaatccatacatctatggtatctataggtcagccgcctggataagtccaaagtcatcaactccgcgttggagctgttgaacgaagttggcattgagggactgacgacccgcaagttggcgcagaagctgggcgtggagcagcccaccctctactggcacgtgaagaataagcgggcgctgctggatgccctggccatcgagatgctcgaccgccaccacacgcatttttgcccgttggaaggcgagtcctggcaggacttcctccgcaataacgccaagtcgttccgctgcgctctgctgtcccaccgagacggtgccaaagtccatctcggcacgcgcccgaccgaaaagcaatacgagacactggagaaccagctcgcgttcctgtgccagcaaggcttcagcctggaaaatgctctctacgctctgagcgccgtcggtcactttaccctgggctgcgtgctggaggaccaagagcatcaagtcgcaaaagaggagcgcgagaccccaacaaccgattcgatgcccccactgctgcgtcaggcaatcgagctgttcgatcatcaaggagccgagccggcattcctgttcggcttggagctgattatctgcggattggaaaagcaactgaaatgcgagtcgggctcgggccccgcctacagccgcgcccgcaccaagaacaactacggcagcaccatcgagggcctgctggatctgccggatgatgatgccccggaggaggcgggcctggccgccccgcgcctgagcttcctgccggccggacacacccgccgcctgtcgaccgccccgccgaccgacgtgagcctgggcgatgagctgcacctggatggcgaggatgtggcgatggcccacgccgatgccctggacgacttcgacctggacatgctgggcgatggcgatagcccgggaccgggattcaccccgcacgatagcgccccctacggcgccctggatatggccgatttcgagttcgagcagatgttcaccgacgccctgggcatcgatgagtacggcggctaa

SEQ ID NO: 7 - DNA, 1348 bp, Ceratitis capitata
atggtaattttaaaagcatatttttttctttgaaattcataagttatcaattatcgatggaaatgtattctatggagaacgttttacccgatgaatgggtgcaaaaattattttaccttcaaatctacaatcaacacacgctaacttttgtgacttgatcaactctcacctggaaaagcaaccaactacaatcaacattctatgggataatcgacaagtgagtaaaattatagccggacctcttagtacagtgtatttaaaaggggaataatattctatcaataggaataaaaataaggtcagcagccatgacttttccatcattttgaatataccttatttgtttcgggattaattgggggtcggaaatcctcttgaattcagaaacgggaaccggaggaaggtgccggtctttcagaaagctgtgaaaaataccaacatttctgctgccaagagctcaataagaagtttcaaaaattgtcttggatgttgcagctgtggctgctaagtaataagacatctattagtatctagatttgttagaccatttaacatagtgttttaaacgatggggttaatagatgagggttaagaagctagttatattactgttgctgtaacgccttcaattgtcggttacagagcaaacattattgaatgttaatgtaaagagtttatttgttttctagtaaacatatagcgattggttagtaatcactaatagaaatttttcataagtatcaaaaaagtaaacctctttttcagtctatgtaataagtaaaccaaggaaagggaaaatatctacaatcaacaagccattgttgcagcaacaaagcaactgaaactacaatcaacattcaataaacttgggtaatttggaatttaattctctgggacacctgtggattacaacaatcaactcgaaacttattatacaatgtaaataaaaattgatatgcatacatgaagatcaagtgaaattccatttagaatcaatttttttcgaatattaagtttcttgctttaatttatctgaaagtaaatagacattccaaattcaagttaacaaattaataatgaattgactagtgatttttaagagaaaaagataagatttaaaaaaggaaagcctttcttgataaatttttgaaccactttatgccgtttcaatcataaaaacttttaagaacacatgactggtaaaattaatttaaaacaaatttaaattttcaacgtaacattcaacaaaaatggtgaaaactatcacggaaattgttaatattaatatgtcccaaaaatagcctttgtatgtatatgatactaatccatacatctatggtatctatag

SEQ ID NO: 8 - DNA, 174 bp, Ceratitis capitata
gtaattttaaaagcatatttttttctttgaaattcataagttatcaattatcgatggaaatgtattctatggagaacgttttacccgatgaatgggtgcaaaaattattttaccttcaaatctacaatcaacacacgctaacttttgtgacttgatcaactctcacctggaaaa

SEQ ID NO: 9 - DNA, 40 bp, Ceratitis capitata
gcaaccaactacaatcaacattctatgggataatcgacaa

SEQ ID NO: 10 - DNA, 521 bp, Ceratitis capitata
gtgagtaaaattatagccggacctcttagtacagtgtatttaaaaggggaataatattctatcaataggaataaaaataaggtcagcagccatgacttttccatcattttgaatataccttatttgtttcgggattaattgggggtcggaaatcctcttgaattcagaaacgggaaccggaggaaggtgccggtctttcagaaagctgtgaaaaataccaacatttctgctgccaagagctcaataagaagtttcaaaaattgtcttggatgttgcagctgtggctgctaagtaataagacatctattagtatctagatttgttagaccatttaacatagtgttttaaacgatggggttaatagatgagggttaagaagctagttatattactgttgctgtaacgccttcaattgtcggttacagagcaaacattattgaatgttaatgtaaagagtttatttgttttctagtaaacatatagcgattggttagtaatcactaatagaaatttttcataag

SEQ ID NO: 11 - DNA, 27 bp, Ceratitis capitata
tatcaaaaaagtaaacctctttttcag

SEQ ID NO: 12 - DNA, 174 bp, Ceratitis capitata
tctatgtaataagtaaaccaaggaaagggaaaatatctacaatcaacaagccattgttgcagcaacaaagcaactgaaactacaatcaacattcaataaacttgggtaatttggaatttaattctctgggacacctgtggattacaacaatcaactcgaaacttattatacaat

SEQ ID NO: 13 - DNA, 409 bp, Ceratitis capitata
gtaaataaaaattgatatgcatacatgaagatcaagtgaaattccatttagaatcaatttttttcgaatattaagtttcttgctttaatttatctgaaagtaaatagacattccaaattcaagttaacaaattaataatgaattgactagtgatttttaagagaaaaagataagatttaaaaaaggaaagcctttcttgataaatttttgaaccactttatgccgtttcaatcataaaaacttttaagaacacatgactggtaaaattaatttaaaacaaatttaaattttcaacgtaacattcaacaaaaatggtgaaaactatcacggaaattgttaatattaatatgtcccaaaaatagcctttgtatgtatatgatactaatccatacatctatggtatctatag

SEQ ID NO: 14 - DNA, 130 bp, Drosophila melanogaster
agcgccggagtataaatagaggcgcttcgtctacggagcgacaattcaattcaaacaagcaaagtgaacacgtcgctaagcgaaagctaagcaaataaacaagcgcagctgaacaagctaaacaatctgc

SEQ ID NO: 15 - DNA, 296 bp, Artificial Sequence
  Synthetic DNA contains 7 repeats of the Tn10 tet-operon
tcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtc

SEQ ID NO: 16 - DNA, 229 bp, Artificial Sequence
  Synthetic DNA non-coding fragment based on Simian virus (SV40)
gatcataatcagccataccacatttgtagaggttttacttgctttaaaaaacctccccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 17 - DNA, 1008 bp, Artificial Sequence
  Synthetic DNA encoding the fusion tetracycline transactivator protein optimised for expression in insects.
gtcagccgcctggacaagagcaaggtgatcaacagcgccctggagctgctgaacgaagtgggcatcgagggcctgaccacccgcaagctggcccagaagctgggcgtggaacagccgaccctgtactggcacgtgaagaacaagcgcgccctgctggacgccctggccatcgaaatgctggatcgccaccacacccacttctgcccgctggagggcgagagctggcaggatttcctgcgcaacaacgccaagagcttccgctgcgccctgctgtcgcaccgcgatggcgccaaggtgcacctgggcacccgcccgaccgagaagcagtacgagaccctggagaaccagctggccttcctgtgccagcagggcttcagcctggagaacgccctgtacgccctgagcgccgtgggccacttcaccctgggctgtgtgctggaggatcaggagcaccaggtggccaaggaggagcgcgagaccccgaccaccgatagcatgccgccgctgctgcgccaggccatcgagctgttcgatcaccagggcgccgagccggccttcctgttcggcctggagctgatcatctgcggcctggaaaagcagctgaagtgcgagagcggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcgggtag

SEQ ID NO: 18 - DNA, 971 bp, Bactrocera zonata
atggtaattctaattacttactaaatatagtgatatttaaaaatgctatgagatcttgaggcgaacttaaatgagacaaggtgttagcttgctcaaatgacgcaccaatcaacttaccaagataatttggagcaatatggaatattttcttgtcaaagaactacaatcaacatgcaactggtataataaagtgagtataaagtagaaaggtgcggtataatatttttctctaaagtattaaggtaagtttttaaaaataatttaataatttttcagagtgtgcagtattagtgtctaaaagtattgcaatctttgtgttttaaaaatttgtgcagaatatcaaaaaataggacgacctattgttagtaatattatgtatatttacttattttcaaataaataaaattattttctagcctttagaaacagccttatactacaatcaacaaaccataaatactccctgcaacaattacaaagcaacacaaacaacaatcaacattccatgaactcggacaatttggaaattgatatcttctggcacatgcggattacaacaatcaactcaaacctattagatgatgtaaataaatataaaccttcatattcaataacttaattcgtgatagaaatttagagttaattactgtttaatcgaaatttgaacgtctcgtttgaattcactttaacgtttgaaagatgatctttaaatagtgactggagacttaaattttaatctttttaattaaataatctaacaaagttctttcttattctaatcttaatatataatcttgttttaaaatttgaattaaacttatttcgttgagtaacaattaaatgatttgaatcgtaaaaaatatcattataattattcaacaaaaagctgaaggctgcgatgaaaaattgttgatatataaatcaaatttatttttataaatcactaatagtgacattaaccgtattcacag

SEQ ID NO: 19 - DNA, 187 bp, Bactrocera zonata
gtaattctaattacttactaaatatagtgatatttaaaaatgctatgagatcttgaggcgaacttaaatgagacaaggtgttagcttgctcaaatgacgcaccaatcaacttaccaagataatttggagcaatatggaatattttcttgtcaaagaactacaatcaacatgcaactggtataataaa

SEQ ID NO: 20 - DNA, 226 bp, Bactrocera zonata
gtgagtataaagtagaaaggtgcggtataatatttttctctaaagtattaaggtaagtttttaaaaataatttaataatttttcagagtgtgcagtattagtgtctaaaagtattgcaatctttgtgttttaaaaatttgtgcagaatatcaaaaaataggacgacctattgttagtaatattatgtatatttacttattttcaaataaataaaattattttctag

SEQ ID NO: 21 - DNA, 167 bp, Bactrocera zonata
cctttagaaacagccttatactacaatcaacaaaccataaatactccctgcaacaattacaaagcaacacaaacaacaatcaacattccatgaactcggacaatttggaaattgatatcttctggcacatgcggattacaacaatcaactcaaacctattagatgat

SEQ ID NO: 22 - DNA, 388 bp, Bactrocera zonata
gtaaataaatataaaccttcatattcaataacttaattcgtgatagaaatttagagttaattactgtttaatcgaaatttgaacgtctcgtttgaattcactttaacgtttgaaagatgatctttaaatagtgactggagacttaaattttaatctttttaattaaataatctaacaaagttctttcttattctaatcttaatatataatcttgttttaaaatttgaattaaacttatttcgttgagtaacaattaaatgatttgaatcgtaaaaaatatcattataattattcaacaaaaagctgaaggctgcgatgaaaaattgttgatatataaatcaaatttatttttataaatcactaatagtgacattaaccgtattcacag

SEQ ID NO: 23 - DNA, 246 bp, Drosophila melanogaster
tctaaacaattattgacggaaagtaccagttctttgccgttcttcgcccatttaccaggaaccccagtaggtaaagtagcggatttcgcgaattttcgcgggtatggcaataaaacaggcagatgttttttaatccccaaaataggtcctttctacctgtgcgcttggcaaagtatataaaggtgtcgcgtcgtccgccagaacttagttgaacatttctgtttcccggagcacatctgatagaac

SEQ ID NO: 24 - DNA, 294 bp, Artificial Sequence
  Synthetic DNA contains 14 repeats of the Tn10 tet-operon
actccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagaga

SEQ ID NO: 25 - DNA, 70 bp, Drosophila melanogaster
gtaagttgatcaaaggaaacgcaaagttttcaagaaaaaacaaaactaatttgatttataacacctttag

SEQ ID NO: 26 - DNA, 393 bp, Artificial Sequence
  Synthetic DNA used as a transcriptional activator
atgggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcggg

SEQ ID NO: 27 - DNA, 21 bp, Artificial Sequence
  Snthetic DNA
attaagttttattatatttac

SEQ ID NO: 28 - DNA, 675 bp, Artificial Sequence
  Synthetic DNA derived from Dicosoma (Clontech)
atggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctg

SEQ ID NO: 29 - DNA, 87 bp, Drosophila melanogaster
ttggggtaagttttcccgttcttttctgggttcttcccttttgctcatccttgctgcactaccttcaggtgcaagttgagattcagg

SEQ ID NO: 30 - DNA, 633 bp, Autographa californica nucleopolyhedrovirus
cttgtcgccgccagtgtcaacttgcaactgaaacaatatccaacatgaacgtcaatttatactgccctaatggcgaacacgataacaatatttcttttattatgccctctaaaaccaacgcggttatcgtttatttattcaaattagatatagaacatccgccgacatacaatgttaatgcaaaaacgcgtttggtgagcggatacgaaaacagtcggccgataaacattaatctgaggtcggtaacaccgtccttgaacggaacacgaggagcgtacgtgatcagctgcattcgcgcgccgcgcctttatcgagatttatttgcatacaacaagtacactgcgccgttgggatttgtggtaacgcgcacacatgcagagctgcaagtgtggcacattttgtctgtgcgcaaaacctttgaagccaaaagtacgaggtccgttacgggcatgctactagcgcacacggacaatggacccgacaaattctacgccaaggatttaatgataatgtcgggcaacgtatccgttcattttatcaataacctacaaaaatgtcgcgcgcatcacaaagacatcgatatatttaaacatttatgtcccgaactgcaaatcgataatagtgttgtgcaac

SEQ ID NO: 31 - DNA, 563 bp, Autographa californica nucleopolyhedrovirus
gctttacgagtagaattctacgcgtaaaacacaatcaagtatgagtcataagctgatgtcatgttttgcacacggctcataaccgaactggctttacgagtagaattctacttgtaacgcacgatcagtggatgatgtcatttgtttttcaaatcgagatgatgtcatgttttgcacacggctcataaactcgctttacgagtagaattctacgtgtaacgcacgatcgattgatgagtcatttgttttgcaatatgatatcatacaatatgactcatttgtttttcaaaaccgaacttgatttacgggtagaattctacttgtaaagcacaatcaaaaagatgatgtcatttgtttttcaaaactgaactcgctttacgagtagaattctacgtgtaaaacacaatcaagaaatgatgtcatttgttataaaaataaaagctgatgtcatgttttgcacatggctcataactaaactcgctttacgggtagaattctacgcgtaaaacatgattgataattaaataattcatttgcaagctatacgttaaatcaaacggacg

SEQ ID NO: 32 - DNA, 221 bp, Artificial Sequence
  Synthetic DNA
tcgcgctcgcgcgactgacggtcgtaagcacccgcgtacgtgtccaccccggtcacaaccccttgtgtcatgtcggcgaccctacgcccccaactgagagaactcaaaggttaccccagttggggcactactcccgaaaaccgcttctgacctgggaaaacgtgaagccccggggcatccgctgagggttgccgccggggcttcggtgtgtccgtcagtac

SEQ ID NO: 33 - DNA, 296 bp, Ceratitis capitata
agtcgattaagttaattagtaaatagatataattccaacgttccaaaaattgaatgcaaattgtcagcttttctacgtcataagttcatttagtttacataatttgtattcgatttagcgaacatcattaatgtggtaatataacaactatgactgcttatgttcaattaatgagtataaaaatagaattttcgttgtcgcgttttaagcggcttcttatatctcaaatcaaaggataacttatgaatcgatgtatgagtatgagaatttacatataaataaatgtttaaattaac

SEQ ID NO: 34 - DNA, 1014 bp, Artificial Sequence
  Synthetic DNA based on a fusion of sequences from E. coli (tetR - tetracycline repressor) and HSV-1 (VP16 transcriptional activator)
atgggcagccgcctggataagtccaaagtcatcaactccgcgttggagctgttgaacgaagttggcattgagggactgacgacccgcaagttggcgcagaagctgggcgtggagcagcccaccctctactggcacgtgaagaataagcgggcgctgctggatgccctggccatcgagatgctcgaccgccaccacacgcatttttgcccgttggaaggcgagtcctggcaggacttcctccgcaataacgccaagtcgttccgctgcgctctgctgtcccaccgagacggtgccaaagtccatctcggcacgcgcccgaccgaaaagcaatacgagacactggagaaccagctcgcgttcctgtgccagcaaggcttcagcctggaaaatgctctctacgctctgagcgccgtcggtcactttaccctgggctgcgtgctggaggaccaagagcatcaagtcgcaaaagaggagcgcgagaccccaacaaccgattcgatgcccccactgctgcgtcaggcaatcgagctgttcgatcatcaaggagccgagccggcattcctgttcggcttggagctgattatctgcggattggaaaagcaactgaaatgcgagtcgggctcgggccccgcctacagccgcgcccgcaccaagaacaactacggcagcaccatcgagggcctgctggatctgccggatgatgatgccccggaggaggcgggcctggccgccccgcgcctgagcttcctgccggccggacacacccgccgcctgtcgaccgccccgccgaccgacgtgagcctgggcgatgagctgcacctggatggcgaggatgtggcgatggcccacgccgatgccctggacgacttcgacctggacatgctgggcgatggcgatagcccgggaccgggattcaccccgcacgatagcgccccctacggcgccctggatatggccgatttcgagttcgagcagatgttcaccgacgccctgggcatcgatgagtacggcggc

SEQ ID NO: 35 - DNA, 144 bp, Ceratitis capitata
atcaattgaattggaaaaatacgcttgaaagcacttttgcgcggagcaacaaagaaagtgttcttaaactattataattgcaagtgattaataaaggaattttatactttgttctacgaagttgatacattgaaataaaactag

SEQ ID NO: 36 - DNA, 81 bp, Ceratitis capitata
ctagaacaatctcgtagcttctacacttttgacatttggtttttgtgcctctataaatagggctgttcgcttgcaaccggc

SEQ ID NO: 37 - DNA, 156 bp, Ceratitis capitata
atattttgaaaagatcttagtcagacgggaacgtgttaaaaattagtttttcaaattgcataacttatccaaggatcagtaaccaactataatatttaaagtgtgaatggaaattcacagtatcttcgactaaagaactgcagttggatccgatag

SEQ ID NO: 38 - DNA, 649 bp, Ceratitis capitata
ggacccatgttacaaggctgcaatcctttcgaaaaccaatagtttcgtttgcagctgtacgtagggagtttgaaaagagagcaggtgtgcaaaacgagtagaaaacaacttttcaacgtcgaaccttccttgtgtttgttgacgggcgtttttgagtatattccatatgacaagtttgtgatccgggtcgagtcgaaaatatcataaaaacataatcacaatcatgaaagctatgcttttgtacccttttctttcgcttgttatattcaagggaccattattaaaaacactttctattttgatctttaaataacagttgtttagtgagcagttggtcaaaaacgttgcttaaagttattgctacgtgagagataattgtactgtgatcaactgaagactgaaagagaaacataataaaatggtaactaaattcaccaaaaatgaatgtgagagatttaaaaaaatattttgatagtcatttatactggcatacaagggcgtgccttgaaggggtttctactggacacactggtactgggaattttaataactttgaagctaaaatactcccattttcatttgtagcttttcgtcaaagtaacaatatatatatatttcaaataatgtatcaaaaattttccatataagc

SEQ ID NO: 39 - DNA, 671 bp, Artificial Sequence
  Synthetic DNA contains 21 repeats of the Tn10 tet-operon
tccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagaga

SEQ ID NO: 40 - DNA, 39 bp, Drosophila melanogaster
ccggagtataaatagaggcgcttcgtctacggagcgaca

SEQ ID NO: 41 - DNA, 1626 bp, Ceratitis capitata
gccggcgcgtttcgatcaaatctaactttattccatatagttggcgtaggttttgccgtataccccttataccgggcagtcggtttttttttaaacattagttgtaacttggtacacaaacagttcacaagcaaaatttttccaaaaatttatagtcttatttttatgctcttcttaaaaattagttgtgacaaaaatttggtttttataaaaaacttcataagggtaagtgaaataaaacgaattacgatttatttttacaaaataacataccaacaaatcaattgtttaaaacgaagcgcataattactttctattggtatactaatcagtttcgataaacttcatttgaaaaaaaattttacttttaaaattctcttttaaagaagcttttgtgaaattcagttctgtaaaattcattgagatgcgattaaaataatgtcgactgcaaggttatggggcgtgttgcaccaaagtgttgcaagtcaaagcggaaattcggcggccacgcctttttggcggccggcgtgtttgtctcgatgtcggatggatgtggccctcctatctggctttaaactaatgccggggcagtctgtcgacaggcaaccaccttgccacgccccacgccttgtaatgacgttatttacatgctgcaacaacactggcagcaagcgcgggcaattaaataaatcatagcggttcacgcggttcggccgaaagaaagatccagttaatctaaagagaaactcttagttctttctttcgttgtcagagtgaaattgtaaataaattacacgtcctgattgatcaactttttgttcgttacaaaacaacgtcgggaatcagattgcaactgttattaattatgggctttgttgcagttgcaaccgcttgtctcctgctgttcaggtcttcatcctggtcggtagggcttgggctttacttttggtttgagactcgggttgctcactgattaagtgacttctctaggacgcaactctgcggcttctggggccaacactcacatgcggggggaacataatccatcacagttgatggctgacatcgtacacaacaaataaatcccatataaaacttccctttccttttcttttcagagtttgtaaaaattttctacaatgagttcaaataatgtaaatgagtgcaagagcctgtggaatggcataatttccatttctgcaaaagatgaaagtcctaaaggtctcactgaaatgtgtaatcatccaaagaggagagcacctcaaaaatgtaagccaataaagtcctgtgcaaagccgcgccgaaaggcagcctgtgccaaggcgactcggcccaaggtcaagtgtgcaccgagtcagaagtgcagcaagcagggacctgtcactaacaacgcctatttgaatttcgtgcgtttcttccgaaagaagcactgtgacttgaagccgcaggaattaattgcagaggccgctaaagcgtgggccgagctcccggagcatagaaaggatagataccgccggatggtacgtattttttttttatactatgaagactattaatttgtcctttcaggcatgcaaggtcaccaccagtgaacgccacaagcgccgacggatttgcaccatggcc

SEQ ID NO: 42 - DNA, 1139 bp, Drosophila melanogaster
gccggcgcgtttcgatcaaatctaactttattccatatagttggcgtaggttttgccgtataccccttataccgggcagtcggtttttttttaaacattagttgtaacttggtacacaaacagttcacaagcaaaatttttccaaaaatttatagtcttatttttatgctcttcttaaaaattagttgtgacaaaaatttggtttttataaaaaacttcataagggtaagtgaaataaaacgaattacgatttatttttacaaaataacataccaacaaatcaattgtttaaaacgaagcgcataattactttctattggtatactaatcagtttcgataaacttcatttgaaaaaaaattttacttttaaaattctcttttaaagaagcttttgtgaaattcagttctgtaaaattcattgagatgcgattaaaataatgtcgactgcaaggttatggggcgtgttgcaccaaagtgttgcaagtcaaagcggaaattcggcggccacgcctttttggcggccggcgtgtttgtctcgatgtcggatggatgtggccctcctatctggctttaaactaatgccggggcagtctgtcgacaggcaaccaccttgccacgccccacgccttgtaatgacgttatttacatgctgcaacaacactggcagcaagcgcgggcaattaaataaatcatagcggttcacgcggttcggccgaaagaaagatccagttaatctaaagagaaactcttagttctttctttcgttgtcagagtgaaattgtaaataaattacacgtcctgattgatcaactttttgttcgttacaaaacaacgtcgggaatcagattgcaactgttattaattatgggctttgttgcagttgcaaccgcttgtctcctgctgttcaggtcttcatcctggtcggtagggcttgggctttacttttggtttgagactcgggttgctcactgattaagtgacttctctaggacgcaactctgcggcttctggggccaacactcacatgcggggggaacataatccatcacagttgatggctgacatcgtacacaacaaataaatcccatataaaacttccctttccttttcttttcagagtttgtaaaaattttctaca

SEQ ID NO: 43 - DNA, 893 bp, Ceratitis capitata
gtaagtgaaataaaacgaattacgatttatttttacaaaataacataccaacaaatcaattgtttaaaacgaagcgcataattactttctattggtatactaatcagtttcgataaacttcatttgaaaaaaaattttacttttaaaattctcttttaaagaagcttttgtgaaattcagttctgtaaaattcattgagatgcgattaaaataatgtcgactgcaaggttatggggcgtgttgcaccaaagtgttgcaagtcaaagcggaaattcggcggccacgcctttttggcggccggcgtgtttgtctcgatgtcggatggatgtggccctcctatctggctttaaactaatgccggggcagtctgtcgacaggcaaccaccttgccacgccccacgccttgtaatgacgttatttacatgctgcaacaacactggcagcaagcgcgggcaattaaataaatcatagcggttcacgcggttcggccgaaagaaagatccagttaatctaaagagaaactcttagttctttctttcgttgtcagagtgaaattgtaaataaattacacgtcctgattgatcaactttttgttcgttacaaaacaacgtcgggaatcagattgcaactgttattaattatgggctttgttgcagttgcaaccgcttgtctcctgctgttcaggtcttcatcctggtcggtagggcttgggctttacttttggtttgagactcgggttgctcactgattaagtgacttctctaggacgcaactctgcggcttctggggccaacactcacatgcggggggaacataatccatcacagttgatggctgacatcgtacacaacaaataaatcccatataaaacttccctttccttttcttttcag

SEQ ID NO: 44 - DNA, 402 bp, Drosophila melanogaster
agtttgtaaaaattttctacaatgagttcaaataatgtaaatgagtgcaagagcctgtggaatggcataatttccatttctgcaaaagatgaaagtcctaaaggtctcactgaaatgtgtaatcatccaaagaggagagcacctcaaaaatgtaagccaataaagtcctgtgcaaagccgcgccgaaaggcagcctgtgccaaggcgactcggcccaaggtcaagtgtgcaccgagtcagaagtgcagcaagcagggacctgtcactaacaacgcctatttgaatttcgtgcgtttcttccgaaagaagcactgtgacttgaagccgcaggaattaattgcagaggccgctaaagcgtgggccgagctcccggagcatagaaaggatagataccgccggatg

SEQ ID NO: 45 - DNA, 87 bp, Drosophila melanogaster
attcaattcaaacaagcaaagtgaacacgtcgctaagcgaaagctaagcaaataaacaagcgcagctgaacaagctaaacaatctgc

SEQ ID NO: 46 - DNA, 225 bp, Drosophila melanogaster
gccggcgcgtttcgatcaaatctaactttattccatatagttggcgtaggttttgccgtataccccttataccgggcagtcggtttttttttaaacattagttgtaacttggtacacaaacagttcacaagcaaaatttttccaaaaatttatagtcttatttttatgctcttcttaaaaattagttgtgacaaaaatttggtttttataaaaaacttcataagg

SEQ ID NO: 47 - DNA, 588 bp, Artificial Sequence
  Codon optimized FokI from Flavobacterium okeanokoites
cagctggtgaagagcgagctggaggaaaagaagtccgagctgcgccacaagctgaagtacgtgccccacgagtacatcgagctgatcgagatcgcccgcaatagcacccaggaccgcatcctggagatgaaggtgatggaattcttcatgaaggtgtacggctaccgcggcaagcacctgggcggcagccgcaagcccgacggagccatctacaccgtgggcagccccatcgattacggcgtgatcgtggataccaaggcctacagcggcggctacaacctgcccattggacaggccgacgagatgcagcgctacgtggaggaaaaccagacccgcaacaagcacatcaaccccaacgagtggtggaaggtgtaccccagcagcgtgaccgagttcaagttcctgttcgtgagcggccacttcaagggcaactacaaggcccagctgacccgcctgaaccacatcaccaactgcaacggagccgtgctgtccgtggaggaactgctgatcggcggcgagatgatcaaggccggcaccctgaccctggaagaagtgcgccgcaagttcaacaacggcgagatcaacttc

SEQ ID NO: 48 - DNA, 96 bp, Ceratitis capitata
atatcaatatgaagagtgcgttaattaatgtatgtttgataaattgttatgtgaaattatacttttcgtaaataaaaatatgcaaaagaagtatac

SEQ ID NO: 49 - DNA, 655 bp, Anastrepha ludens
gtcttttgattgtgaaagatggtgaaatgcgttgatgcaaacatatgccaacaacaacagcagcagcaacatcaacaataacaacaactctgcaaatagccagcacaactgctcagacttcgtgcaacgaacaccaataaccagcataacaaataccactacgcattcgagaactgcaattacaacaacaaaagaggcattaatagcaacaacaaaaatgagagtgcatgctaattccaacaaaagcaaaaactacagatatcaacaaggtaatgagaattcaacaaatgcaataaatggcgcttccagtagcgaaagagaaggacacaaaaacaaaaccaaatatataaagaaagcaatctctgaggaaatgcccatgagcgccatctagcaattgaatcgctttacgttgcatttgcatgtgaacatttttaattatatttctttttctggattttatacaataaaatgcgtgtaaaactgaaatttataaatttaactaacagaatatcaaaggaaaattatgaaaatgtagaaaaaaattattaaaaattactaaaatccaaacaaatgaacatcaaataattcactaataagagctaattaagtaaacatctttccattgactaaccaaggcaaaaatactaaaagtaaa

SEQ ID NO: 50 - DNA, 675 bp, Artificial Sequence; identical to SEQ ID NO: 28
  Synthetic DNA encoding variant of red protein from Discocoma (Clontech)

SEQ ID NO: 51 - DNA, 2246 bp, Anastrepha ludens
gacccttgtgccccctgcatattttgatttattttaagctgaactagttctacattttcaaacttagcgcagctcatacatgtacatatctatatatgtaagtatgtatgtatatacgttcgtgtgcatgtgcagtgtgcatgcactgtgcgcaataaaacctgctggaactcatttacaaagcaaaaaattattctttcccatcgattacagcattaaaatcagcgtaagtatgtaaacacgccccatttaagaatccaaaaatacttgcactccatttccatagtgcagccaaatgcatggctatttgtgtgtatttaatgtatgcatatatgttcgtatatttgtatgtatgaatttatgactttatattcaactccactaaaaattgcttttcaccattcggtgggtattcacacattttttacatatatttcatatatcaacgctccagaaatgccgcttgttttactccaaaaggatgcggtgtgccggaaattaagaaatcctgctgtccagccaatttatgtatgccactggaacggcaataacgcgtggccatgtaaacaaatgtgggatcaacaagcaaaacgctggccaagcaaagaaattggttagagaacaaaaataaaaagaaacaagcaattcgaaagctatcatctcaacaacaattacagcaacaatacgagcaaagcgcgcagatcaaagtggcagcacctcgaaaaatccacagaggtggcaacatggagatgaccaaaaatagaaatgtttaacacacagaaaaaggcgccaacaacaaaacaacgtcataaagtattcaacgaaggtgctgagcgcctctacatgcacgtacatacacacaaaaacataaaaattcctacattatgatacatacatacatacataggtgcttacaagtgtacctacatatttattagtatccaatacatacaaatgcaaatttagataccagtgcgtgggggccgccacagctgagcgagagcaatacgcaatgtgagagcaccagcaaccgaaaatatatatttaataaacatatttgaaaaattaaatgttcgaaaatagaatattcaaaatatattttgtttgaaaagcgacaaaatgatcaccgtccggatctattgaagcgcctgacaatgcgtgcaaaagggatagcgattgcgtcagtatgcgcggcaataaacctcaaacacttcatatctctccctccacacacaacccgaaaagatcgcgcgaagcaagacgaggaattcaacaaacaatagcaacaaacacatctgcaatgcttgtatattgaatataaatttgttaaagaaatttcgtgcgcaccccaccacagcatccccattatggtgagcgcttaatagcagccgacataacgttgacgcggcagccttgccttatacggcgcttttggagcgcttttagtggggagccgtaccaattgccatggaatatatatatttgtatgtatgtatgtatgtatgcacaaacaaacccgatgatgccgaactaaacgcgagcaatctgtgagatcagcgaacagcgagcagcgaataacgagcgaaccacccaacaaagtgattatgatcattgctgtgacgctatgacgccacggctcgcttcgttcgtgggctgagtggggtgcttcagtgcgcaggagcacgatcgttgttgctgactcgctcggatgcgcgcacatcgttttccatgcagctcaacattaacgtgtgggtgtgtatgcgtatatacggcagatgacgatgagcgcggtgtgatgggtctatttttagtggcgttgaaaaatcgtaaacacaacgaatccctctttgatgtgaacgtattggtgttgccgctattatctgtcattgtatcagtggatcggtgtatcggtgttttcgtgttggcggtggaggtgagacatttgttggagcgttgcgggtgtgtggggacgtgaagcgacgtgaatgcctaatgttggctattgccgaagaattttttggatttgttaagtgatcaacatgaaaagtgtagttttaagttgattggtataaaatcggctggtagtagccaaggcactttagtcagtcagttgtgttccaaagcgttcagaacacattgccatatttctttacttatttcttcttgctcgtggttaaaaaacacaaaactcagacaaaatgtgtgacgatgaggttgctgctttggtcgttgataatggttccggt

SEQ ID NO: 52 - DNA, 183 bp, Ceratitis capitata
gatgtctagacaaaattgaaaatcaacttttaacgatttgatgtatagtttacatgttctgaaaaattgaaaataaacaaagaataaatattgcgtgtaattaataataatatggcttaaatgaatatgtgagatctccacagctttcaaggttcatttgcttatcagagttctaaaataatg

SEQ ID NO: 53 - DNA, 693 bp, Artificial Sequence
  Synthetic DNA encoding variant of green protein from Zoanthus (Clontech)
atggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccc

SEQ ID NO: 54 - DNA, 761 bp, Ceratitis capitata
tgtatatcattttaaaaccatttgcgcaaatacatttgaaaatgttttcaatcgtacaaattctagtgaaaacgatatacatagttataaaaaatttgttgtattagacagaaaatttaaataaaatcttttatataggaaagttaatccaaattattgcgtccatttttaaaaattgattttcaaattaaaaaaaaaggacttggcatggaggttcaaaaaaccttttcgtcgcgctctacaacaaaacccaaaggcaactcgaaaactgccttttataggaccaaaactggaaaatgtcaacgtcccagcggtattcttcttcggaatccatacctgaattttttacgtgaatttcgcgtgcgcaatacaggtctttcagccgtagaaataatacgacgcggtgccaaggaatggaataatatgcccaaagaggataaattgcattacatagaagaggtttgcctttgttttgctttctctattttgtttaatataattatatatcataggcttttcatgcaccaaagaaaaggaagccaccgtcaattcaaatacaaccacaaccaaaccagatgtgtgcaatgccatatcaaagtgatcgtggtatagtgtgccccagaatgcctaccgcgtgccccaaaaagcgccgcaggaaggcgaaatgtgctcgcaggcggaagcccaaaaaaaggcgatgtggcaaaaaacgccgtcctaagcgcagagtttgcaagccgaggcgaaaaagatgtaaaata

SEQ ID NO: 55 - DNA, 207 bp, Ceratitis capitata
tgtatatcattttaaaaccatttgcgcaaatacatttgaaaatgttttcaatcgtacaaattctagtgaaaacgatatacatagttataaaaaatttgttgtattagacagaaaatttaaataaaatcttttatataggaaagttaatccaaattattgcgtccatttttaaaaattgattttcaaattaaaaaaaaaggacttggc

SEQ ID NO: 56 - DNA, 459 bp, Ceratitis capitata
tgtatatcattttaaaaccatttgcgcaaatacatttgaaaatgttttcaatcgtacaaattctagtgaaaacgatatacatagttataaaaaatttgttgtattagacagaaaatttaaataaaatcttttatataggaaagttaatccaaattattgcgtccatttttaaaaattgattttcaaattaaaaaaaaaggacttggcatggaggttcaaaaaaccttttcgtcgcgctctacaacaaaacccaaaggcaactcgaaaactgccttttataggaccaaaactggaaaatgtcaacgtcccagcggtattcttcttcggaatccatacctgaattttttacgtgaatttcgcgtgcgcaatacaggtctttcagccgtagaaataatacgacgcggtgccaaggaatggaataatatgcccaaagaggataaattgcattacatagaagag

SEQ ID NO: 57 - DNA, 53 bp, Ceratitis capitata
gtttgcctttgttttgctttctctattttgtttaatataattatatatcatag

SEQ ID NO: 58 - DNA, 249 bp, Ceratitis capitata
gcttttcatgcaccaaagaaaaggaagccaccgtcaattcaaatacaaccacaaccaaaccagatgtgtgcaatgccatatcaaagtgatcgtggtatagtgtgccccagaatgcctaccgcgtgccccaaaaagcgccgcaggaaggcgaaatgtgctcgcaggcggaagcccaaaaaaaggcgatgtggcaaaaaacgccgtcctaagcgcagagtttgcaagccgaggcgaaaaagatgtaaaata

SEQ ID NO: 59 - DNA, 1489 bp, Ceratitis capitata
agttctttcttacgcggtattgcagttttgtcgttttctcatttatgtcttatacgcggtattgaaaaaaattatattcatacaagtggattgatttacagaaattgttattaaaaatacatagttttaccaaaactaaataaaattggcatttagtagcatgtttaattaaattaaattaatataaaaatatattatcaggaggtacataaattatataaaaatctttacctatcggccggtataaaattattaagcatcggcaaaaataaaatttatttttaattcttttcaaaaattatttgtgaagtatgcattttttgaatttcactttgtataggcttgtaccgactgatgagtattttagaacccacgagcgaacatcattatctacctcatcagctcgtagattgctcaataggatttcagaccaaatcttgctttcgaattataaaattttgccttttgaatgcaatatatcagaatattgcgctccacaccataaacaccaccttgtgattgtgatgacgactcaagtacctctcttccgtcactaaattgtgcaaataatagatatattcattcttccttatgctttgcattgaatgtattctcgtcgctaaatacgactattttcccctccgtccaaatgacgagctgatttacccatgttcgtatatgcatctggcttgatcggacacaagctccaatacaaccgattactcagatatgtaattttgcttcataaatactttaataatgcatctaagaacacgaacgattgtatgctatgatgtttcgggacacaaattcgtcagagcaaagaatacaaatgtgtgaaattcagtattatccacacatagacatttaaatttaaataagcattgacaccgaaaatcattaaaatcggataatttcatcatatagcccccttacaaccgatttgttcagatatgaatatttggttaagaaaaattcaacaataaaataaaataccataagtttgataagatcataaagactttgttcggcatgacccaaacgaaaattcaccttgaagcaagcaaagaaatttattcttcacaactacctaaactatcttcacccttttccatcgctggctttaaagatagcagggacaatattctaccctcttctaaaatcatctccaaacaaaaaactccaccccatccttcgcaaaaaaaaactttaaaaaacagcatcatcgaaccaagtcaaggtaccaatccatatgtacatacataatttataaaagtacattgcatactaatactggttcaaatatcttaaaaacggaaagtttgaataagtttttaagacacttatttatctctatgcgcaaaaagatggttcttgtagacacttttgctgttggagtttttattcttttaaagtttataaaaagttaaatgttaaaccatctgaaaaaattcatcgaatgttttacactttacacatttaatacttt

SEQ ID NO: 60 - DNA, 296 bp, Ceratitis capitata; identical to SEQ ID NO: 33

SEQ ID NO: 61 - DNA, 1014 bp, Artificial Sequence; identical to SEQ ID NO: 34
  Synthetic DNA based on a fusion of sequences from E. coli (tetR - tetracycline repressor) and HSV-1 (VP16 transcriptional activator)

SEQ ID NO: 62 - DNA, 144 bp, Ceratitis capitata; identical to SEQ ID NO: 35

SEQ ID NO: 63 - DNA, 81 bp, Ceratitis capitata
gccggttgcaagcgaacagccctatttatagaggcacaaaaaccaaatgtcaaaagtgtagaagctacgagattgttctag

SEQ ID NO: 64 - DNA, 156 bp, Ceratitis capitata; identical to SEQ ID NO: 37

SEQ ID NO: 65 - DNA, 649 bp, Ceratitis capitata; identical to SEQ ID NO: 38

SEQ ID NO: 66 - DNA, 671 bp, Artificial Sequence; identical to SEQ ID NO: 39
  Synthetic DNA contains 21 repeats of the Tn10 tet-operon

SEQ ID NO: 67 - DNA, 60 bp, Drosophila melanogaster
ccggagtataaatagaggcgcttcgtctacggagcgacaattcaattcaaacaagcaaag

SEQ ID NO: 68 - DNA, 685 bp, Ceratitis capitata
atacatagttataaaaaatttgttgtattagacagaaaatttaaataaaatcttttatataggaaagttaatccaaattattgcgtccatttttaaaaattgattttcaaattaaaaaaaaaggacttggcatggaggttcaaaaaaccttttcgtcgcgctctacaacaaaacccaaaggcaactcgaaaactgccttttataggaccaaaactggaaaatgtcaacgtcccagcggtattcttcttcggaatccatacctgaattttttacgtgaatttcgcgtgcgcaatacaggtctttcagccgtagaaataatacgacgcggtgccaaggaatggaataatatgcccaaagaggataaattgcattacatagaagaggtttgcctttgttttgctttctctattttgtttaatataattatatatcataggcttttcatgcaccaaagaaaaggaagccaccgtcaattcaaatacaaccacaaccaaaccagatgtgtgcaatgccatatcaaagtgatcgtggtatagtgtgccccagaatgcctaccgcgtgccccaaaaagcgccgcaggaaggcgaaatgtgctcgcaggcggaagcccaaaaaaaggcgatgtggcaaaaaacgccgtcctaagcgcagagtttgcaagccgaggcgaaaaagatgtaaaata

SEQ ID NO: 69 - DNA, 131 bp, Ceratitis capitata
atacatagttataaaaaatttgttgtattagacagaaaatttaaataaaatcttttatataggaaagttaatccaaattattgcgtccatttttaaaaattgattttcaaattaaaaaaaaaggacttggc

SEQ ID NO: 70 - DNA, 252 bp, Ceratitis capitata
atggaggttcaaaaaaccttttcgtcgcgctctacaacaaaacccaaaggcaactcgaaaactgccttttataggaccaaaactggaaaatgtcaacgtcccagcggtattcttcttcggaatccatacctgaattttttacgtgaatttcgcgtgcgcaatacaggtctttcagccgtagaaataatacgacgcggtgccaaggaatggaataatatgcccaaagaggataaattgcattacatagaagag

SEQ ID NO: 71 - DNA, 53 bp, Ceratitis capitata; identical to SEQ ID NO: 57

SEQ ID NO: 72 - DNA, 249 bp, Ceratitis capitata; identical to SEQ ID NO: 58

SEQ ID NO: 73 - DNA, 588 bp, Artificial Sequence; identical to SEQ ID NO: 47
  Codon optimized FokI from Flavobacterium okeanokoites

SEQ ID NO: 74 - DNA, 183 bp, Ceratitis capitata; identical to SEQ ID NO: 52

SEQ ID NO: 75 - DNA, 655 bp, Anastrepha ludens; identical to SEQ ID NO: 49

SEQ ID NO: 76 - DNA, 675 bp, Artificial Sequence; identical to SEQ ID NO: 28
  Synthetic DNA derived from Dicosoma (Clontech)

SEQ ID NO: 77 - DNA, 2246 bp, Anastrepha ludens; identical to SEQ ID NO: 51

SEQ ID NO: 78 - DNA, 183 bp, Ceratitis capitata; identical to SEQ ID NO: 52

SEQ ID NO: 79 - DNA, 693 bp, Artificial Sequence; identical to SEQ ID NO: 53
  Synthetic DNA encoding variant of green protein from Zoanthus (Clontech)

SEQ ID NO: 80 - DNA, 761 bp, Ceratitis capitata; identical to SEQ ID NO: 54

SEQ ID NO: 81 - DNA, 207 bp, Ceratitis capitata; identical to SEQ ID NO: 55

SEQ ID NO: 82 - DNA, 252 bp, Ceratitis capitata; identical to SEQ ID NO: 70

SEQ ID NO: 83 - DNA, 53 bp, Ceratitis capitata; identical to SEQ ID NO: 57

SEQ ID NO: 84 - DNA, 249 bp, Ceratitis capitata; identical to SEQ ID NO: 58

SEQ ID NO: 85 - DNA, 1489 bp, Ceratitis capitata; identical to SEQ ID NO: 59

SEQ ID NO: 86 - DNA, 1014 bp, Artificial Sequence
  Synthetic DNA based on a fusion of sequences from E. coli (tetR - tetracycline repressor) and HSV-1 (VP16 transcriptional activator)
atggtcagccgcctggataagtccaaagtcatcaactccgcgttggagctgttgaacgaagttggcattgagggactgacgacccgcaagttggcgcagaagctgggcgtggagcagcccaccctctactggcacgtgaagaataagcgggcgctgctggatgccctggccatcgagatgctcgaccgccaccacacgcatttttgcccgttggaaggcgagtcctggcaggacttcctccgcaataacgccaagtcgttccgctgcgctctgctgtcccaccgagacggtgccaaagtccatctcggcacgcgcccgaccgaaaagcaatacgagacactggagaaccagctcgcgttcctgtgccagcaaggcttcagcctggaaaatgctctctacgctctgagcgccgtcggtcactttaccctgggctgcgtgctggaggaccaagagcatcaagtcgcaaaagaggagcgcgagaccccaacaaccgattcgatgcccccactgctgcgtcaggcaatcgagctgttcgatcatcaaggagccgagccggcattcctgttcggcttggagctgattatctgcggattggaaaagcaactgaaatgcgagtcgggctcgggccccgcctacagccgcgcccgcaccaagaacaactacggcagcaccatcgagggcctgctggatctgccggatgatgatgccccggaggaggcgggcctggccgccccgcgcctgagcttcctgccggccggacacacccgccgcctgtcgaccgccccgccgaccgacgtgagcctgggcgatgagctgcacctggatggcgaggatgtggcgatggcccacgccgatgccctggacgacttcgacctggacatgctgggcgatggcgatagcccgggaccgggattcaccccgcacgatagcgccccctacggcgccctggatatggccgatttcgagttcgagcagatgttcaccgacgccctgggcatcgatgagtacggcggc

SEQ ID NO: 87 - DNA, 1008 bp, Artificial Sequence
  Synthetic DNA based on a fusion of sequences from E. coli (tetR - tetracycline repressor) and HSV-1 (VP16 transcriptional activator)
atggtcagccgcctggacaagagcaaggtgatcaacagcgccctggagctgctgaacgaagtgggcatcgagggcctgaccacccgcaagctggcccagaagctgggcgtggaacagccgaccctgtactggcacgtgaagaacaagcgcgccctgctggacgccctggccatcgaaatgctggatcgccaccacacccacttctgcccgctggagggcgagagctggcaggatttcctgcgcaacaacgccaagagcttccgctgcgccctgctgtcgcaccgcgatggcgccaaggtgcacctgggcacccgcccgaccgagaagcagtacgagaccctggagaaccagctggccttcctgtgccagcagggcttcagcctggagaacgccctgtacgccctgagcgccgtgggccacttcaccctgggctgtgtgctggaggatcaggagcaccaggtggccaaggaggagcgcgagaccccgaccaccgatagcatgccgccgctgctgcgccaggccatcgagctgttcgatcaccagggcgccgagccggccttcctgttcggcctggagctgatcatctgcggcctggaaaagcagctgaagtgcgagagcggcagcgcctacagccgcgcccgtaccaagaacaactatggcagcaccatcgagggactgctggacctgccggatgacgatgccccggaggaagccggcctggccgccccccgcctgagcttcctgcccgccggacacacgcgccgcctgagcaccgccccgccgaccgatgtgagcctgggcgacgagctgcacctggatggagaggatgtggcaatggcccacgccgacgccctggacgatttcgacctggatatgctgggcgatggagatagcccgggaccgggcttcacgccccacgatagcgccccgtacggcgccctggacatggccgacttcgagttcgagcaaatgttcaccgacgcgctgggcatcgatgagtatggcggg

SEQ ID NO: 88 - protein, 338 aa, Artificial Sequence
  Synthetic protein based on a fusion of sequences from E. coli (tetR - tetracycline repressor) and HSV-1 (VP16 transcriptional activator)
MGSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSGPAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 89 - protein, 338 aa, Artificial Sequence
  Synthetic protein based on a fusion of sequences from E. coli (tetR - tetracycline repressor) and HSV-1 (VP16 transcriptional activator)
MVSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSGPAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 90 - protein, 336 aa, Artificial Sequence
  Synthetic protein based on a fusion of sequences from E. coli (tetR - tetracycline repressor) and HSV-1 (VP16 transcriptional activator)
MVSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 91 - DNA, 49 bp, Drosophila melanogaster
gtacgtattttttttttatactatgaagactattaatttgtcctttcag

SEQ ID NO: 92 - DNA, 57 bp, Drosophila melanogaster
gcatgcaaggtcaccaccagtgaacgccacaagcgccgacggatttgcaccatggcc

SEQ ID NO: 93 - DNA, 1626 bp, Drosophila melanogaster; identical to SEQ ID NO: 41

SEQ ID NO: 94 - DNA, 11380 bp, Artificial Sequence
  Expression vector unit for integration
gttaatttaaacatttatttatatgtaaattctcatactcatacatcgattcataagttatcctttgatttgagatataagaagccgcttaaaacgcgacaacgaaaattctatttttatactcattaattgaacataagcagtcatagttgttatattaccacattaatgatgttcgctaaatcgaatacaaattatgtaaactaaatgaacttatgacgtagaaaagctgacaatttgcattcaatttttggaacgttggaattatatctatttactaattaacttaatcgactcactcattagccgccgtactcatcgatgcccagggcgtcggtgaacatctgctcgaactcgaaatcggccatatccagggcgccgtagggggcgctatcgtgcggggtgaatcccggtcccgggctatcgccatcgcccagcatgtccaggtcgaagtcgtccagggcatcggcgtgggccatcgccacatcctcgccatccaggtgcagctcatcgcccaggctcacgtcggtcggcggggcggtcgacaggcggcgggtgtgtccggccggcaggaagctcaggcgcggggcggccaggcccgcctcctccggggcatcatcatccggcagatccagcaggccctcgatggtgctgccgtagttgttcttggtgcgggcgcggctgtaggcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgcccatggtggtttactagttttatttcaatgtatcaacttcgtagaacaaagtataaaattcctttattaatcacttgcaattataatagtttaagaacactttctttgttgctccgcgcaaaagtgctttcaagcgtatttttccaattcaattgatgccggttgcaagcgaacagccctatttatagaggcacaaaaaccaaatgtcaaaagtgtagaagctacgagattgttctagctatcggatccaactgcagttctttagtcgaagatactgtgaatttccattcacactttaaatattatagttggttactgatccttggataagttatgcaatttgaaaaactaatttttaacacgttcccgtctgactaagatcttttcaaaatatgcttatatggaaaatttttgatacattatttgaaatatatatatattgttactttgacgaaaagctacaaatgaaaatgggagtattttagcttcaaagttattaaaattcccagtaccagtgtgtccagtagaaaccccttcaaggcacgcccttgtatgccagtataaatgactatcaaaatatttttttaaatctctcacattcatttttggtgaatttagttaccattttattatgtttctctttcagtcttcagttgatcacagtacaattatctctcacgtagcaataactttaagcaacgtttttgaccaactgctcactaaacaactgttatttaaagatcaaaatagaaagtgtttttaataatggtcccttgaatataacaagcgaaagaaaagggtacaaaagcatagctttcatgattgtgattatgtttttatgatattttcgactcgacccggatcacaaacttgtcatatggaatatactcaaaaacgcccgtcaacaaacacaaggaaggttcgacgttgaaaagttgttttctactcgttttgcacacctgctctcttttcaaactccctacgtacagctgcaaacgaaactattggttttcgaaaggattgcagccttgtaacatgggtccggtccgaccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatcgcgcgcgaccggagtataaatagaggcgcttcgtctacggagcgacaattcaattcaaacaagcaaagatacatagttataaaaaatttgttgtattagacagaaaatttaaataaaatcttttatataggaaagttaatccaaattattgcgtccatttttaaaaattgattttcaaattaaaaaaaaaggacttggcatggaggttcaaaaaaccttttcgtcgcgctctacaacaaaacccaaaggcaactcgaaaactgccttttataggaccaaaactggaaaatgtcaacgtcccagcggtattcttcttcggaatccatacctgaattttttacgtgaatttcgcgtgcgcaatacaggtctttcagccgtagaaataatacgacgcggtgccaaggaatggaataatatgcccaaagaggataaattgcattacatagaagaggtttgcctttgttttgctttctctattttgtttaatataattatatatcataggcttttcatgcaccaaagaaaaggaagccaccgtcaattcaaatacaaccacaaccaaaccagatgtgtgcaatgccatatcaaagtgatcgtggtatagtgtgccccagaatgcctaccgcgtgccccaaaaagcgccgcaggaaggcgaaatgtgctcgcaggcggaagcccaaaaaaaggcgatgtggcaaaaaacgccgtcctaagcgcagagtttgcaagccgaggcgaaaaagatgtaaaatacagctggtgaagagcgagctggaggaaaagaagtccgagctgcgccacaagctgaagtacgtgccccacgagtacatcgagctgatcgagatcgcccgcaatagcacccaggaccgcatcctggagatgaaggtgatggaattcttcatgaaggtgtacggctaccgcggcaagcacctgggcggcagccgcaagcccgacggagccatctacaccgtgggcagccccatcgattacggcgtgatcgtggataccaaggcctacagcggcggctacaacctgcccattggacaggccgacgagatgcagcgctacgtggaggaaaaccagacccgcaacaagcacatcaaccccaacgagtggtggaaggtgtaccccagcagcgtgaccgagttcaagttcctgttcgtgagcggccacttcaagggcaactacaaggcccagctgacccgcctgaaccacatcaccaactgcaacggagccgtgctgtccgtggaggaactgctgatcggcggcgagatgatcaaggccggcaccctgaccctggaagaagtgcgccgcaagttcaacaacggcgagatcaacttctaagatgtctagacaaaattgaaaatcaacttttaacgatttgatgtatagtttacatgttctgaaaaattgaaaataaacaaagaataaatattgcgtgtaattaataataatatggcttaaatgaatatgtgagatctccacagctttcaaggttcatttgcttatcagagttctaaaataatgcgccggcgatccgaggaaatggaacgtttacttttagtatttttgccttggttagtcaatggaaagatgtttacttaattagctcttattagtgaattatttgatgttcatttgtttggattttagtaatttttaataatttttttctacattttcataattttcctttgatattctgttagttaaatttataaatttcagttttacacgcattttattgtataaaatccagaaaaagaaatataattaaaaatgttcacatgcaaatgcaacgtaaagcgattcaattgctagatggcgctcatgggcatttcctcagagattgctttctttatatatttggttttgtttttgtgtccttctctttcgctactggaagcgccatttattgcatttgttgaattctcattaccttgttgatatctgtagtttttgcttttgttggaattagcatgcactctcatttttgttgttgctattaatgcctcttttgttgttgtaattgcagttctcgaatgcgtagtggtatttgttatgctggttattggtgttcgttgcacgaagtctgagcagttgtgctggctatttgcagagttgttgttattgttgatgttgctgctgctgttgttgttggcatatgtttgcatcaacgcatttcaccatctttcacaatcaaaagactcacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtaccacggcgcgccataccggaaccattatcaacgaccaaagcagcaacctcatcgtcacacattttgtctgagttttgtgttttttaaccacgagcaagaagaaataagtaaagaaatatggcaatgtgttctgaacgctttggaacacaactgactgactaaagtgccttggctactaccagccgattttataccaatcaacttaaaactacacttttcatgttgatcacttaacaaatccaaaaaattcttcggcaatagccaacattaggcattcacgtcgcttcacgtccccacacacccgcaacgctccaacaaatgtctcacctccaccgccaacacgaaaacaccgatacaccgatccactgatacaatgacagataatagcggcaacaccaatacgttcacatcaaagagggattcgttgtgtttacgatttttcaacgccactaaaaatagacccatcacaccgcgctcatcgtcatctgccgtatatacgcatacacacccacacgttaatgttgagctgcatggaaaacgatgtgcgcgcatccgagcgagtcagcaacaacgatcgtgctcctgcgcactgaagcaccccactcagcccacgaacgaagcgagccgtggcgtcatagcgtcacagcaatgatcataatcactttgttgggtggttcgctcgttattcgctgctcgctgttcgctgatctcacagattgctcgcgtttagttcggcatcatcgggtttgtttgtgcatacatacatacatacatacaaatatatatattccatggcaattggtacggctccccactaaaagcgctccaaaagcgccgtataaggcaaggctgccgcgtcaacgttatgtcggctgctattaagcgctcaccataatggggatgctgtggtggggtgcgcacgaaatttctttaacaaatttatattcaatatacaagcattgcagatgtgtttgttgctattgtttgttgaattcctcgtcttgcttcgcgcgatcttttcgggttgtgtgtggagggagagatatgaagtgtttgaggtttattgccgcgcatactgacgcaatcgctatcccttttgcacgcattgtcaggcgcttcaatagatccggacggtgatcattttgtcgcttttcaaacaaaatatattttgaatattctattttcgaacatttaatttttcaaatatgtttattaaatatatattttcggttgctggtgctctcacattgcgtattgctctcgctcagctgtggcggcccccacgcactggtatctaaatttgcatttgtatgtattggatactaataaatatgtaggtacacttgtaagcacctatgtatgtatgtatgtatcataatgtaggaatttttatgtttttgtgtgtatgtacgtgcatgtagaggcgctcagcaccttcgttgaatactttatgacgttgttttgttgttggcgcctttttctgtgtgttaaacatttctatttttggtcatctccatgttgccacctctgtggatttttcgaggtgctgccactttgatctgcgcgctttgctcgtattgttgctgtaattgttgttgagatgatagctttcgaattgcttgtttctttttatttttgttctctaaccaatttctttgcttggccagcgttttgcttgttgatcccacatttgtttacatggccacgcgttattgccgttccagtggcatacataaattggctggacagcaggatttcttaatttccggcacaccgcatccttttggagtaaaacaagcggcatttctggagcgttgatatatgaaatatatgtaaaaaatgtgtgaatacccaccgaatggtgaaaagcaatttttagtggagttgaatataaagtcataaattcatacatacaaatatacgaacatatatgcatacattaaatacacacaaatagccatgcatttggctgcactatggaaatggagtgcaagtatttttggattcttaaatggggcgtgtttacatacttacgctgattttaatgctgtaatcgatgggaaagaataattttttgctttgtaaatgagttccagcaggttttattgcgcacagtgcatgcacactgcacatgcacacgaacgtatatacatacatacttacatatatagatatgtacatgtatgagctgcgctaagtttgaaaatgtagaactagttcagcttaaaataaatcaaaatatgcagggggcacaagggtccattattttagaactctgataagcaaatgaaccttgaaagctgtggagatctcacatattcatttaagccatattattattaattacacgcaatatttattctttgtttattttcaatttttcagaacatgtaaactatacatcaaatcgttaaaagttgattttcaattttgtctagacatcttagggcccttagagtccggagggcaaggcggagccggaggcgatggcgtgctcggtcaggtgccacttctggttcttggcgtcgctgcggtcctcgcgggtcagcttgtgctggatgaagtgccagtcgggcatcttgcggggcacggacttggccttgtacacggtgtcgaactggcagcgcaagcggccaccgtccttcagcagcaggtacatgctcacgtcgcccttcaagatgccctgcttgggcacggggatgatcttctcgcaggagggctcccagttgtcggtcatcttcttcatcacggggccgtcggcggggaagttcacgccgtagaacttggactcgtggtacatgcagttctcctccacgctcacggtgatgtcggcgttgcagatgcacacggcgccgtcctcgaacaggaaggagcggtcccaggtgtagccggcggggcaggagttcttgaagtagtcgacgatgtcctgggggtactcggtgaacacgcggttgccgtacatgaaggcggcggacaagatgtcctcggcgaagggcaaggggccgccctccaccacgcacaggttgatggcctgcttgcccttgaaggggtagccgatgccctcgccggtgatcacgaacttgtggccgtccacgcagccctccatgcggtacttcatggtcatctccttggtcaggccgtgcttggactgggccatggtggaccggtatcgtattttacatctttttcgcctcggcttgcaaactctgcgcttaggacggcgttttttgccacatcgcctttttttgggcttccgcctgcgagcacatttcgccttcctgcggcgctttttggggcacgcggtaggcattctggggcacactataccacgatcactttgatatggcattgcacacatctggtttggttgtggttgtatttgaattgacggtggcttccttttctttggtgcatgaaaagcctatgatatataattatattaaacaaaatagagaaagcaaaacaaaggcaaacctcttctatgtaatgcaatttatcctctttgggcatattattccattccttggcaccgcgtcgtattatttctacggctgaaagacctgtattgcgcacgcgaaattcacgtaaaaaattcaggtatggattccgaagaagaataccgctgggacgttgacattttccagttttggtcctataaaaggcagttttcgagttgcctttgggttttgttgtagagcgcgacgaaaaggttttttgaacctccatgccaagtcctttttttttaatttgaaaatcaatttttaaaaatggacgcaataatttggattaactttcctatataaaagattttatttaaattttctgtctaatacaacaaattttttataactatgtatatcgttttcactagaatttgtacgattgaaaacattttcaaatgtatttgcgcaaatggttttaaaatgatatacaaaagtattaaatgtgtaaagtgtaaaacattcgatgaattttttcagatggtttaacatttaactttttataaactttaaaagaataaaaactccaacagcaaaagtgtctacaagaaccatctttttgcgcatagagataaataagtgtcttaaaaacttattcaaactttccgtttttaagatatttgaaccagtattagtatgcaatgtacttttataaattatgtatgtacatatggattggtaccttgacttggttcgatgatgctgttttttaaagtttttttttgcgaaggatggggtggagttttttgtttggagatgattttagaagagggtagaatattgtccctgctatctttaaagccagcgatggaaaagggtgaagatagtttaggtagttgtgaagaataaatttctttgcttgcttcaaggtgaattttcgtttgggtcatgccgaacaaagtctttatgatcttatcaaacttatggtattttattttattgttgaatttttcttaaccaaatattcatatctgaacaaatcggttgtaagggggctatatgatgaaattatccgattttaatgattttcggtgtcaatgcttatttaaatttaaatgtctatgtgtggataatactgaatttcacacatttgtattctttgctctgacgaatttgtgtcccgaaacatcatagcatacaatcgttcgtgttcttagatgcattattaaagtatttatgaagcaaaattacatatctgagtaatcggttgtattggagcttgtgtccgatcaagccagatgcatatacgaacatgggtaaatcagctcgtcatttggacggaggggaaaatagtcgtatttagcgacgagaatacattcaatgcaaagcataaggaagaatgaatatatctattatttgcacaatttagtgacggaagagaggtacttgagtcgtcatcacaatcacaaggtggtgtttatggtgtggagcgcaatattctgatatattgcattcaaaaggcaaaattttataattcgaaagcaagatttggtctgaaatcctattgagcaatctacgagctgatgaggtagataatgatgttcgctcgtgggttctaaaatactcatcagtcggtacaagcctatacaaagtgaaattcaaaaaatgcatacttcacaaataatttttgaaaagaattaaaaataaattttatttttgccgatgcttaataattttataccggccgataggtaaagatttttatataatttatgtacctcctgataatatatttttatattaatttaatttaattaaacatgctactaaatgccaattttatttagttttggtaaaactatgtatttttaataacaatttctgtaaatcaatccacttgtatgaatataatttttttcaataccgcgtataagacataaatgagaaaacgacaaaactgcaataccgcgtaagaaagaact

SEQ ID NO: 95 - DNA, 12420 bp, Artificial Sequence
  Expression vector unit for integration
ttaacctaggttaatttaaacatttatttatatgtaaattctcatactcatacatcgattcataagttatcctttgatttgagatataagaagccgcttaaaacgcgacaacgaaaattctatttttatactcattaattgaacataagcagtcatagttgttatattaccacattaatgatgttcgctaaatcgaatacaaattatgtaaactaaatgaacttatgacgtagaaaagctgacaatttgcattcaatttttggaacgttggaattatatctatttactaattaacttaatcgactcactcattagccgccgtactcatcgatgcccagggcgtcggtgaacatctgctcgaactcgaaatcggccatatccagggcgccgtagggggcgctatcgtgcggggtgaatcccggtcccgggctatcgccatcgcccagcatgtccaggtcgaagtcgtccagggcatcggcgtgggccatcgccacatcctcgccatccaggtgcagctcatcgcccaggctcacgtcggtcggcggggcggtcgacaggcggcgggtgtgtccggccggcaggaagctcaggcgcggggcggccaggcccgcctcctccggggcatcatcatccggcagatccagcaggccctcgatggtgctgccgtagttgttcttggtgcgggcgcggctgtaggcggggcccgagcccgactcgcatttcagttgcttttccaatccgcagataatcagctccaagccgaacaggaatgccggctcggctccttgatgatcgaacagctcgattgcctgacgcagcagtgggggcatcgaatcggttgttggggtctcgcgctcctcttttgcgacttgatgctcttggtcctccagcacgcagcccagggtaaagtgaccgacggcgctcagagcgtagagagcattttccaggctgaagccttgctggcacaggaacgcgagctggttctccagtgtctcgtattgcttttcggtcgggcgcgtgccgagatggactttggcaccgtctcggtgggacagcagagcgcagcggaacgacttggcgttattgcggaggaagtcctgccaggactcgccttccaacgggcaaaaatgcgtgtggtggcggtcgagcatctcgatggccagggcatccagcagcgcccgcttattcttcacgtgccagtagagggtgggctgctccacgcccagcttctgcgccaacttgcgggtcgtcagtccctcaatgccaacttcgttcaacagctccaacgcggagttgatgactttggacttatccaggcggctgcccatggtggtttactagttttatttcaatgtatcaacttcgtagaacaaagtataaaattcctttattaatcacttgcaattataatagtttaagaacactttctttgttgctccgcgcaaaagtgctttcaagcgtatttttccaattcaattgatgccggttgcaagcgaacagccctatttatagaggcacaaaaaccaaatgtcaaaagtgtagaagctacgagattgttctagctatcggatccaactgcagttctttagtcgaagatactgtgaatttccattcacactttaaatattatagttggttactgatccttggataagttatgcaatttgaaaaactaatttttaacacgttcccgtctgactaagatcttttcaaaatatgcttatatggaaaatttttgatacattatttgaaatatatatatattgttactttgacgaaaagctacaaatgaaaatgggagtattttagcttcaaagttattaaaattcccagtaccagtgtgtccagtagaaaccccttcaaggcacgcccttgtatgccagtataaatgactatcaaaatatttttttaaatctctcacattcatttttggtgaatttagttaccattttattatgtttctctttcagtcttcagttgatcacagtacaattatctctcacgtagcaataactttaagcaacgtttttgaccaactgctcactaaacaactgttatttaaagatcaaaatagaaagtgtttttaataatggtcccttgaatataacaagcgaaagaaaagggtacaaaagcatagctttcatgattgtgattatgtttttatgatattttcgactcgacccggatcacaaacttgtcatatggaatatactcaaaaacgcccgtcaacaaacacaaggaaggttcgacgttgaaaagttgttttctactcgttttgcacacctgctctcttttcaaactccctacgtacagctgcaaacgaaactattggttttcgaaaggattgcagccttgtaacatgggtccggtccgaccggcgctcgttttcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgaaacctggcgcgccccggccatcgagaaagagagagagaagagaagagagagaacattcgagaaagagagagagaagagaagagagagaacatactccctatcagtgatagagaagtccctatcagtgatagagatgtccctatcagtgatagagagttccctatcagtgatagagacgtccctatcagtgatagagaagtccctatcagtgatagagagatccctatcagtgatagagatttccctatcagtgatagagaggtccctatcagtgatagagacttccctatcagtgatagagaaatccctatcagtgatagagacatccctatcagtgatagagaactccctatcagtgatagagacctccctatcagtgatagagatcgatcgcgcgcgaccggagtataaatagaggcgcttcgtctacggagcgacaattcaattcaaacaagcaaagtgaacacgtcgctaagcgaaagctaagcaaataaacaagcgcagctgaacaagctaaacaatctgcaggtacctgccggcgcgtttcgatcaaatctaactttattccatatagttggcgtaggttttgccgtataccccttataccgggcagtcggtttttttttaaacattagttgtaacttggtacacaaacagttcacaagcaaaatttttccaaaaatttatagtcttatttttatgctcttcttaaaaattagttgtgacaaaaatttggtttttataaaaaacttcataagggtaagtgaaataaaacgaattacgatttatttttacaaaataacataccaacaaatcaattgtttaaaacgaagcgcataattactttctattggtatactaatcagtttcgataaacttcatttgaaaaaaaattttacttttaaaattctcttttaaagaagcttttgtgaaattcagttctgtaaaattcattgagatgcgattaaaataatgtcgactgcaaggttatggggcgtgttgcaccaaagtgttgcaagtcaaagcggaaattcggcggccacgcctttttggcggccggcgtgtttgtctcgatgtcggatggatgtggccctcctatctggctttaaactaatgccggggcagtctgtcgacaggcaaccaccttgccacgccccacgccttgtaatgacgttatttacatgctgcaacaacactggcagcaagcgcgggcaattaaataaatcatagcggttcacgcggttcggccgaaagaaagatccagttaatctaaagagaaactcttagttctttctttcgttgtcagagtgaaattgtaaataaattacacgtcctgattgatcaactttttgttcgttacaaaacaacgtcgggaatcagattgcaactgttattaattatgggctttgttgcagttgcaaccgcttgtctcctgctgttcaggtcttcatcctggtcggtagggcttgggctttacttttggtttgagactcgggttgctcactgattaagtgacttctctaggacgcaactctgcggcttctggggccaacactcacatgcggggggaacataatccatcacagttgatggctgacatcgtacacaacaaataaatcccatataaaacttccctttccttttcttttcagagtttgtaaaaattttctacaatgagttcaaataatgtaaatgagtgcaagagcctgtggaatggcataatttccatttctgcaaaagatgaaagtcctaaaggtctcactgaaatgtgtaatcatccaaagaggagagcacctcaaaaatgtaagccaataaagtcctgtgcaaagccgcgccgaaaggcagcctgtgccaaggcgactcggcccaaggtcaagtgtgcaccgagtcagaagtgcagcaagcagggacctgtcactaacaacgcctatttgaatttcgtgcgtttcttccgaaagaagcactgtgacttgaagccgcaggaattaattgcagaggccgctaaagcgtgggccgagctcccggagcatagaaaggatagataccgccggatggtacgtattttttttttatactatgaagactattaatttgtcctttcaggcatgcaaggtcaccaccagtgaacgccacaagcgccgacggatttgcaccatggcccagctggtgaagagcgagctggaggaaaagaagtccgagctgcgccacaagctgaagtacgtgccccacgagtacatcgagctgatcgagatcgcccgcaatagcacccaggaccgcatcctggagatgaaggtgatggaattcttcatgaaggtgtacggctaccgcggcaagcacctgggcggcagccgcaagcccgacggagccatctacaccgtgggcagccccatcgattacggcgtgatcgtggataccaaggcctacagcggcggctacaacctgcccattggacaggccgacgagatgcagcgctacgtggaggaaaaccagacccgcaacaagcacatcaaccccaacgagtggtggaaggtgtaccccagcagcgtgaccgagttcaagttcctgttcgtgagcggccacttcaagggcaactacaaggcccagctgacccgcctgaaccacatcaccaactgcaacggagccgtgctgtccgtggaggaactgctgatcggcggcgagatgatcaaggccggcaccctgaccctggaagaagtgcgccgcaagttcaacaacggcgagatcaacttctaacccatatcaatatgaagagtgcgttaattaatgtatgtttgataaattgttatgtgaaattatacttttcgtaaataaaaatatgcaaaagaagtatacccaaaaagcaattaagtgtacttactaaccatgattcaataattaaagcttggataaataaacatttttctctctccctcttaacaaaaaggcgtatcttaacgcttaagtttacttttagtatttttgccttggttagtcaatggaaagatgtttacttaattagctcttattagtgaattatttgatgttcatttgtttggattttagtaatttttaataatttttttctacattttcataattttcctttgatattctgttagttaaatttataaatttcagttttacacgcattttattgtataaaatccagaaaaagaaatataattaaaaatgttcacatgcaaatgcaacgtaaagcgattcaattgctagatggcgctcatgggcatttcctcagagattgctttctttatatatttggttttgtttttgtgtccttctctttcgctactggaagcgccatttattgcatttgttgaattctcattaccttgttgatatctgtagtttttgcttttgttggaattagcatgcactctcatttttgttgttgctattaatgcctcttttgttgttgtaattgcagttctcgaatgcgtagtggtatttgttatgctggttattggtgttcgttgcacgaagtctgagcagttgtgctggctatttgcagagttgttgttattgttgatgttgctgctgctgttgttgttggcatatgtttgcatcaacgcatttcaccatctttcacaatcaaaagactcacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtaccacggcgcgccataccggaaccattatcaacgaccaaagcagcaacctcatcgtcacacattttgtctgagttttgtgttttttaaccacgagcaagaagaaataagtaaagaaatatggcaatgtgttctgaacgctttggaacacaactgactgactaaagtgccttggctactaccagccgattttataccaatcaacttaaaactacacttttcatgttgatcacttaacaaatccaaaaaattcttcggcaatagccaacattaggcattcacgtcgcttcacgtccccacacacccgcaacgctccaacaaatgtctcacctccaccgccaacacgaaaacaccgatacaccgatccactgatacaatgacagataatagcggcaacaccaatacgttcacatcaaagagggattcgttgtgtttacgatttttcaacgccactaaaaatagacccatcacaccgcgctcatcgtcatctgccgtatatacgcatacacacccacacgttaatgttgagctgcatggaaaacgatgtgcgcgcatccgagcgagtcagcaacaacgatcgtgctcctgcgcactgaagcaccccactcagcccacgaacgaagcgagccgtggcgtcatagcgtcacagcaatgatcataatcactttgttgggtggttcgctcgttattcgctgctcgctgttcgctgatctcacagattgctcgcgtttagttcggcatcatcgggtttgtttgtgcatacatacatacatacatacaaatatatatattccatggcaattggtacggctccccactaaaagcgctccaaaagcgccgtataaggcaaggctgccgcgtcaacgttatgtcggctgctattaagcgctcaccataatggggatgctgtggtggggtgcgcacgaaatttctttaacaaatttatattcaatatacaagcattgcagatgtgtttgttgctattgtttgttgaattcctcgtcttgcttcgcgcgatcttttcgggttgtgtgtggagggagagatatgaagtgtttgaggtttattgccgcgcatactgacgcaatcgctatcccttttgcacgcattgtcaggcgcttcaatagatccggacggtgatcattttgtcgcttttcaaacaaaatatattttgaatattctattttcgaacatttaatttttcaaatatgtttattaaatatatattttcggttgctggtgctctcacattgcgtattgctctcgctcagctgtggcggcccccacgcactggtatctaaatttgcatttgtatgtattggatactaataaatatgtaggtacacttgtaagcacctatgtatgtatgtatgtatcataatgtaggaatttttatgtttttgtgtgtatgtacgtgcatgtagaggcgctcagcaccttcgttgaatactttatgacgttgttttgttgttggcgcctttttctgtgtgttaaacatttctatttttggtcatctccatgttgccacctctgtggatttttcgaggtgctgccactttgatctgcgcgctttgctcgtattgttgctgtaattgttgttgagatgatagctttcgaattgcttgtttctttttatttttgttctctaaccaatttctttgcttggccagcgttttgcttgttgatcccacatttgtttacatggccacgcgttattgccgttccagtggcatacataaattggctggacagcaggatttcttaatttccggcacaccgcatccttttggagtaaaacaagcggcatttctggagcgttgatatatgaaatatatgtaaaaaatgtgtgaatacccaccgaatggtgaaaagcaatttttagtggagttgaatataaagtcataaattcatacatacaaatatacgaacatatatgcatacattaaatacacacaaatagccatgcatttggctgcactatggaaatggagtgcaagtatttttggattcttaaatggggcgtgtttacatacttacgctgattttaatgctgtaatcgatgggaaagaataattttttgctttgtaaatgagttccagcaggttttattgcgcacagtgcatgcacactgcacatgcacacgaacgtatatacatacatacttacatatatagatatgtacatgtatgagctgcgctaagtttgaaaatgtagaactagttcagcttaaaataaatcaaaatatgcagggggcacaagggtccattattttagaactctgataagcaaatgaaccttgaaagctgtggagatctcacatattcatttaagccatattattattaattacacgcaatatttattctttgtttattttcaatttttcagaacatgtaaactatacatcaaatcgttaaaagttgattttcaattttgtctagacatcttagggcccttagagtccggagggcaaggcggagccggaggcgatggcgtgctcggtcaggtgccacttctggttcttggcgtcgctgcggtcctcgcgggtcagcttgtgctggatgaagtgccagtcgggcatcttgcggggcacggacttggccttgtacacggtgtcgaactggcagcgcaagcggccaccgtccttcagcagcaggtacatgctcacgtcgcccttcaagatgccctgcttgggcacggggatgatcttctcgcaggagggctcccagttgtcggtcatcttcttcatcacggggccgtcggcggggaagttcacgccgtagaacttggactcgtggtacatgcagttctcctccacgctcacggtgatgtcggcgttgcagatgcacacggcgccgtcctcgaacaggaaggagcggtcccaggtgtagccggcggggcaggagttcttgaagtagtcgacgatgtcctgggggtactcggtgaacacgcggttgccgtacatgaaggcggcggacaagatgtcctcggcgaagggcaaggggccgccctccaccacgcacaggttgatggcctgcttgcccttgaaggggtagccgatgccctcgccggtgatcacgaacttgtggccgtccacgcagccctccatgcggtacttcatggtcatctccttggtcaggccgtgcttggactgggccatggtggaccggtatcgtattttacatctttttcgcctcggcttgcaaactctgcgcttaggacggcgttttttgccacatcgcctttttttgggcttccgcctgcgagcacatttcgccttcctgcggcgctttttggggcacgcggtaggcattctggggcacactataccacgatcactttgatatggcattgcacacatctggtttggttgtggttgtatttgaattgacggtggcttccttttctttggtgcatgaaaagcctatgatatataattatattaaacaaaatagagaaagcaaaacaaaggcaaacctcttctatgtaatgcaatttatcctctttgggcatattattccattccttggcaccgcgtcgtattatttctacggctgaaagacctgtattgcgcacgcgaaattcacgtaaaaaattcaggtatggattccgaagaagaataccgctgggacgttgacattttccagttttggtcctataaaaggcagttttcgagttgcctttgggttttgttgtagagcgcgacgaaaaggttttttgaacctccatgccaagtcctttttttttaatttgaaaatcaatttttaaaaatggacgcaataatttggattaactttcctatataaaagattttatttaaattttctgtctaatacaacaaattttttataactatgtatatcgttttcactagaatttgtacgattgaaaacattttcaaatgtatttgcgcaaatggttttaaaatgatatacaaaagtattaaatgtgtaaagtgtaaaacattcgatgaattttttcagatggtttaacatttaactttttataaactttaaaagaataaaaactccaacagcaaaagtgtctacaagaaccatctttttgcgcatagagataaataagtgtcttaaaaacttattcaaactttccgtttttaagatatttgaaccagtattagtatgcaatgtacttttataaattatgtatgtacatatggattggtaccttgacttggttcgatgatgctgttttttaaagtttttttttgcgaaggatggggtggagttttttgtttggagatgattttagaagagggtagaatattgtccctgctatctttaaagccagcgatggaaaagggtgaagatagtttaggtagttgtgaagaataaatttctttgcttgcttcaaggtgaattttcgtttgggtcatgccgaacaaagtctttatgatcttatcaaacttatggtattttattttattgttgaatttttcttaaccaaatattcatatctgaacaaatcggttgtaagggggctatatgatgaaattatccgattttaatgattttcggtgtcaatgcttatttaaatttaaatgtctatgtgtggataatactgaatttcacacatttgtattctttgctctgacgaatttgtgtcccgaaacatcatagcatacaatcgttcgtgttcttagatgcattattaaagtatttatgaagcaaaattacatatctgagtaatcggttgtattggagcttgtgtccgatcaagccagatgcatatacgaacatgggtaaatcagctcgtcatttggacggaggggaaaatagtcgtatttagcgacgagaatacattcaatgcaaagcataaggaagaatgaatatatctattatttgcacaatttagtgacggaagagaggtacttgagtcgtcatcacaatcacaaggtggtgtttatggtgtggagcgcaatattctgatatattgcattcaaaaggcaaaattttataattcgaaagcaagatttggtctgaaatcctattgagcaatctacgagctgatgaggtagataatgatgttcgctcgtgggttctaaaatactcatcagtcggtacaagcctatacaaagtgaaattcaaaaaatgcatacttcacaaataatttttgaaaagaattaaaaataaattttatttttgccgatgcttaataattttataccggccgataggtaaagatttttatataatttatgtacctcctgataatatatttttatattaatttaatttaattaaacatgctactaaatgccaattttatttagttttggtaaaactatgtatttttaataacaatttctgtaaatcaatccacttgtatgaatataatttttttcaataccgcgtataagacataaatgagaaaacgacaaaactgcaataccgcgtaagaaagaactacgcggccgcttttaa

SEQ ID NO: 96 - protein, 225 aa, Artificial Sequence
  A variant of red fluorescent protein originally identified in Discosoma (Clontech)
MASSENVITEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFL

SEQ ID NO: 97 - DNA, 1089 bp, Artificial Sequence
  Exons 2 and 3 of Ceratitis capitata Protamine fused to FokI coding region
atggaggttcaaaaaaccttttcgtcgcgctctacaacaaaacccaaaggcaactcgaaaactgccttttataggaccaaaactggaaaatgtcaacgtcccagcggtattcttcttcggaatccatacctgaattttttacgtgaatttcgcgtgcgcaatacaggtctttcagccgtagaaataatacgacgcggtgccaaggaatggaataatatgcccaaagaggataaattgcattacatagaagaggcttttcatgcaccaaagaaaaggaagccaccgtcaattcaaatacaaccacaaccaaaccagatgtgtgcaatgccatatcaaagtgatcgtggtatagtgtgccccagaatgcctaccgcgtgccccaaaaagcgccgcaggaaggcgaaatgtgctcgcaggcggaagcccaaaaaaaggcgatgtggcaaaaaacgccgtcctaagcgcagagtttgcaagccgaggcgaaaaagatgtaaaatacagctggtgaagagcgagctggaggaaaagaagtccgagctgcgccacaagctgaagtacgtgccccacgagtacatcgagctgatcgagatcgcccgcaatagcacccaggaccgcatcctggagatgaaggtgatggaattcttcatgaaggtgtacggctaccgcggcaagcacctgggcggcagccgcaagcccgacggagccatctacaccgtgggcagccccatcgattacggcgtgatcgtggataccaaggcctacagcggcggctacaacctgcccattggacaggccgacgagatgcagcgctacgtggaggaaaaccagacccgcaacaagcacatcaaccccaacgagtggtggaaggtgtaccccagcagcgtgaccgagttcaagttcctgttcgtgagcggccacttcaagggcaactacaaggcccagctgacccgcctgaaccacatcaccaactgcaacggagccgtgctgtccgtggaggaactgctgatcggcggcgagatgatcaaggccggcaccctgaccctggaagaagtgcgccgcaagttcaacaacggcgagatcaacttc

SEQ ID NO: 98 - protein, 363 aa, Artificial Sequence
  Fusion of Exons 2 and 3 of Ceratitis capitata Protamine with FokI
MEVQKTFSSRSTTKPKGNSKTAFYRTKTGKCQRPSGILLRNPYLNFLREFRVRNTGLSAVEIIRRGAKEWNNMPKEDKLHYIEEAFHAPKKRKPPSIQIQPQPNQMCAMPYQSDRGIVCPRMPTACPKKRRRKAKCARRRKPKKRRCGKKRRPKRRVCKPRRKRCKIQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 99 - DNA, 1026 bp, Artificial Sequence
  Exons 2 and 3 of Drosophila melanogaster Protamine B fused to FokI coding region
atgagttcaaataatgtaaatgagtgcaagagcctgtggaatggcataatttccatttctgcaaaagatgaaagtcctaaaggtctcactgaaatgtgtaatcatccaaagaggagagcacctcaaaaatgtaagccaataaagtcctgtgcaaagccgcgccgaaaggcagcctgtgccaaggcgactcggcccaaggtcaagtgtgcaccgagtcagaagtgcagcaagcagggacctgtcactaacaacgcctatttgaatttcgtgcgtttcttccgaaagaagcactgtgacttgaagccgcaggaattaattgcagaggccgctaaagcgtgggccgagctcccggagcatagaaaggatagataccgccggatggcatgcaaggtcaccaccagtgaacgccacaagcgccgacggatttgcaccatggcccagctggtgaagagcgagctggaggaaaagaagtccgagctgcgccacaagctgaagtacgtgccccacgagtacatcgagctgatcgagatcgcccgcaatagcacccaggaccgcatcctggagatgaaggtgatggaattcttcatgaaggtgtacggctaccgcggcaagcacctgggcggcagccgcaagcccgacggagccatctacaccgtgggcagccccatcgattacggcgtgatcgtggataccaaggcctacagcggcggctacaacctgcccattggacaggccgacgagatgcagcgctacgtggaggaaaaccagacccgcaacaagcacatcaaccccaacgagtggtggaaggtgtaccccagcagcgtgaccgagttcaagttcctgttcgtgagcggccacttcaagggcaactacaaggcccagctgacccgcctgaaccacatcaccaactgcaacggagccgtgctgtccgtggaggaactgctgatcggcggcgagatgatcaaggccggcaccctgaccctggaagaagtgcgccgcaagttcaacaacggcgagatcaacttc

SEQ ID NO: 100 - protein, 342 aa, Artificial Sequence
  Fusion of Exons 2 and 3 of D. melanogaster ProtamineB with FokI
MSSNNVNECKSLWNGIISISAKDESPKGLTEMCNHPKRRAPQKCKPIKSCAKPRRKAACAKATRPKVKCAPSQKCSKQGPVTNNAYLNFVRFFRKKHCDLKPQELIAEAAKAWAELPEHRKDRYRRMACKVTTSERHKRRRICTMAQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 101 - protein, 196 aa, Flavobacterium okeanokoites
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 102 - protein, 336 aa, Artificial Sequence
  Fusion of sequences from E. coli (tetR - tetracycline repressor) and HSV-1 (VP16 transcriptional activator)
MELRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

(19) United States
(12) Patent Application Publication
(10) Pub. No.: US 2021/0324409 A1
(43) Pub. Date: Oct. 21, 2021

(54) Self-Selecting Sterile Male Arthropods

(71) Applicant: Oxitec Limited, Abingdon (GB)

(72) Inventors: Ryan Turkel, Abingdon (GB); Martha Koukidou, Abingdon (GB); Tarig Dafa'alla, Abingdon (GB); Nathan Rose, Abingdon (GB); Romisa Asadi, Abingdon (GB); Simon Warner, Abingdon (GB)

(21) Appl. No.: 17/267,645

(22) PCT Filed: Aug. 13, 2019
(86) PCT No.: PCT/GB2019/052272

§ 371 Date: Feb. 10, 2021

Related U.S. Application Data
(60) Provisional application No. 62/718,555, filed on Aug. 14, 2018.

(51) Int. Cl. C12N 15/85; A01K 67/033

(52) U.S. Cl. CPC C12N 15/8509; A01K 67/0339; A01K 2227/706; C12N 2840/002; A01K 2217/075; C12N 2830/42

(57) ABSTRACT

The invention provides a gene expression system that imparts homozygous, sex-specific lethality in arthropods, particularly Tephritid insects, such as Ceratitis capitata. The arthropod strains produce males that are also engineered to be sterile. The sterile may be released to mate with wild female to suppress propagation of the arthropod population.

### CROSS-REFERENCE TO RELATED APPLICATIONS

This Application is a National Stage Application of PCT Application No. PCT/GB2019/052272, filed on Aug. 13, 2019, and claims benefit of U.S. Provisional Application No. 62/718,555, filed Aug. 14, 2018. The disclosure of each is hereby incorporated by reference in its entirety.

### REFERENCE TO SEQUENCE LISTING

This application incorporates by reference a “Sequence Listing” (identified below) which is submitted concurrently herewith in text file format via the U.S. Patent Office's Electronic Filing System (EFS). The text file copy of the Sequence Listing submitted herewith is labeled “INX00443_ST25.txt”, is a file of 128,173 bytes in size, and was created on Aug. 9, 2019; this Sequence Listing is incorporated by reference in its entirety herein.

### BACKGROUND OF THE INVENTION

The Mediterranean fruit fly Ceratitis capitata is one of the world's most destructive agricultural pests, affecting more than 250 fruit and vegetables and is a major quarantine pest for the US, European and Japanese markets (Diamantidis et al. (2008) J. Appl. Entomol. 132:695-705. Female medflies lay their eggs in fruit, and developing larvae feed within the fruit causing premature drop and decay. Current pest control measures are still highly dependent on the use of insecticides and/or the use of the Sterile Insect Technique (SIT). The SIT involves the rearing and mass-release of self-limiting male insects, to suppress the population of established pest species (Alphey, L. et al. (2002) Insect Biochem. Mol. Biol. 32:1243-1247; Alphey and Andreasen (2002) Mol Biochem Parasitol. 121(2):173-8; Ant, T. et al. (2012) BMC Biol. 10:51; Fu G. et al. (2007) Nat Biotechnol. 25(3):353-7; Gong, F. et al. (2005) Nat Biotechnol. 23(4):453-456; Leftwich, P. T. et al. (2014) Proc Biol Sci. 281(1792) 20141372; Morrison, N. et al. (2010) Asia Pacif J. Mol. Biol. Biotechnol. 18(2):275-295). Radiation, the conventional method for inducing reproductive sterility to released insects, can have a negative impact on their mating performance and longevity in the field, which leads to higher operational costs (Hafez and Shoukry (1972) Z. Ang. Entomol. 72:59-66, (Robinson et al. (2002) Florida Entomologist 85(1):171-181.

There is a need in the art for a biological solution to control Ceratitis capitata that allows the sterile male flies to compete with wild males for mating and therefore be more effective in limiting the propagation of Medflies.

### BRIEF SUMMARY OF THE INVENTION

The invention provides a gene expression system for controlled expression of an effector gene in an arthropod comprising:
(a) a first expression unit comprising:
   i. a first promoter that functions in an arthropod operably linked to a 5′UTR/CDS gene sequence;
   ii. an effector gene operably linked to said 5′UTR/CDS;
   iii. a 3′UTR operably linked to the effector gene; and
   iv. a tetracycline repressible element operably linked to the promoter, wherein transcription of the effector gene is repressed by tetracycline or a tetracycline analog;
(b) a second expression unit comprising a coding sequence for a transcription factor operably linked to an upstream regulatory element, in which the transcription factor is capable of acting on the first promoter of the first expression unit to drive expression of an effector gene, wherein the upstream regulatory element comprises:
   i. a first promoter/5′UTR comprising an arthropod gene promoter operably linked to a corresponding arthropod gene 5′UTR;
   ii. a second promoter/5′UTR operably linked to the first promoter/5′UTR wherein the second promoter/5′UTR is adjacent to a start site for the transcription of the transcription factor coding sequence;
   wherein said first promoter/5′UTR and said second promoter/5′UTR together have testes specificity; and wherein the upstream regulatory element drives sufficient expression of the transcription factor such that the transcription factor in turn drives transcription of the effector gene; and
(c) at least one third expression unit comprising:
   i. a heterologous polynucleotide encoding a functional protein, the coding sequence of which is defined between a start codon and a stop codon;
   ii. a second promoter capable of initiating transcription in the arthropod operably linked to the heterologous polynucleotide; and
   iii. a splice control polynucleotide which, in cooperation with a spliceosome in the arthropod, is capable of sex-specifically mediating in the arthropod
      (A) a first splicing of an RNA transcript of the heterologous polynucleotide to produce a first spliced mRNA product, which does not have a continuous open reading frame extending from said start codon to the stop codon; and
      (B) an alternative splicing of the RNA transcript to yield an alternatively spliced mRNA product which comprises a continuous open reading frame extending from the start codon to the stop codon, wherein said functional protein has a lethal effect on the arthropod wherein said third expression unit is repressible.

In some embodiments, the gene expression system is an inducible system, where induction or repression occurs by provision or absence of a chemical entity, such as, but not limited to tetracycline or an analogue thereof.

In some embodiments, the first promoter is a minimal promoter. In some embodiments, the first promoter is an HSP70 minipro promoter, a mini p35 promoter, a mini CMV promoter (CMVm), an Ac5 promoter, a polyhedron promoter, or a UAS promoter.

In some embodiments, the 5′UTR/CDS sequence is testes-specific. In some embodiments, the 5′UTR/CDS sequence is a protamine 5′UTR/CDS or 5′ Protamine B gene sequence. In some embodiments, the 5′UTR/CDS is a Ceratitis capitata Protamine 5′UTR/CDS or a Drosophila melanogaster Protamine B 5′UTR/CDS. In some embodiments, the 5′UTR/CDS gene sequence comprises a polynucleotide sequence that is 80%, 85%, 90%, 95%, 98% or 100% identical to SEQ ID NO:41, SEQ ID NO:69 or SEQ ID NO:93.

In some embodiments, the 3′UTR is testes-specific. In some embodiments, the 3′UTR is from the same gene as the 5′UTR/CDS gene sequence. In some embodiments, the 3′UTR is a protamine or protamine-like 3′UTR. In certain embodiments, the 3′UTR comprises a polynucleotide sequence that is 80%, 85%, 90%, 95%, 98% or 100% identical to SEQ ID NO:52 or SEQ ID NO:48.

In some embodiments, the effector gene encodes a nuclease or an interfering RNA. In some embodiments, nuclease is a 3-Zn finger nuclease. In certain embodiments, the 3-Zn finger nuclease is a FokI nuclease. In some embodiments, the FokI nuclease is the endonuclease domain of FokI without a DNA-binding domain. In certain embodiments, the FokI nuclease has a polypeptide sequence that is 80%, 85%, 90%, 95%, 98% or 100% identical to SEQ ID NO:101.

In some embodiments, the first promoter/5′UTR comprises a topi, aly or β-tubulin promoter or homologue thereof, operably linked to a corresponding topi, aly or β-tubulin 5′UTR.

In some embodiments, the transcription factor is a heterologous transcriptional activator. In some embodiments, the transcription factor in the second expression unit is tTA or a variant thereof. In some embodiments, tTA is tTAV, tTAV2, or tTAV3.

In some embodiments, the transcription factor of the second expression unit is tTA or a variant thereof, and the first expression unit comprises a tet operator (tetO).

In some embodiments, the functional protein is an apoptosis-inducing factor, Hid, Reaper (Rpr), or NipplDm.

In some embodiments, the RNA transcript comprises two or more coding exons for the functional protein. In some embodiments, the functional protein is conditionally suppressible.

In some embodiments of the gene expression system, the third expression unit comprises at least one positive feedback mechanism, having at least one functional protein to be differentially expressed, via alternative splicing, and at least one promoter therefor, wherein a product of a gene to be expressed serves as a positive transcriptional control factor for the at least one promoter therefor, and whereby the expression of said product is suppressible.

In some embodiments, an enhancer is associated with the second promoter, and the functional protein enhances activity of said second promoter via the enhancer. In some embodiments, one or more tetO operator units are operably linked with the promoter and act as the enhancer. The tTA or its analogue serves to enhance activity of the promoter via tetO.

In some embodiments, the functional protein itself a transcriptional transactivator, such as the tTAV system, (e.g., tTAV, tTAV2 or tTAV3).

In some embodiments, the first expression unit is activated by the presence or absence of a chemical entity. In some embodiments, the second expression unit is activated by the presence or absence of a chemical entity. In some embodiments, the third expression unit is activated by the presence or absence of a chemical entity. In some embodiments, a plurality of expression units are activated by the presence or absence of a chemical entity (e.g., first and second; second and third; first and third; first, second and third). In some embodiments, the chemical entity is tetracycline or an analog thereof.

In some embodiments of the gene expression system, the second promoter is a srya embryo-specific promoter, or a homologue thereof, a Drosophila Hsp70 (e.g., a DmHsp70) promoter or a homologue thereof, or a Drosophila slow as molasses (slam) promoter or a homologue thereof.

In some embodiments, the splice control polynucleotide is derived from the Ceratitis capitata transformer gene (Cctra), the Drosophila transformer gene (e.g., from D. melanogaster (Dmtra); D. suzukii (Dstra); etc.), the Ceratitis rosa transformer gene (Crtra), or the Bactrocera zonata transformer gene (Bztra). In some embodiments, the splice control polynucleotide is derived from a Drosophila spp. doublesex (dsx) gene, Bombyx mori dsx gene, Pink Boll Worm dsx gene, Ceratitis capitata dsx gene, Codling Moth dsx gene, or a mosquito dsx gene, such as from an Aedes spp. (e.g., Aedes gambiae, Aedes aegypti, etc.).

In some embodiments of the gene expression system, at least one splice control polynucleotide comprises an intron and wherein said intron comprises on its 5′ end, a guanine (G) nucleotide, in RNA. In some embodiments, the splice control polynucleotide comprises an intron and wherein said intron comprises on its 5′ end, UG nucleotides, and UT at its 3′ end, in RNA. In some embodiments, the system comprises a consensus core sequence of WWCRAT, where W=A or T, and R=A or G.

In some embodiments, the arthropod is an insect. In some embodiments, the insect is a Tephritid. In certain embodiments, the Tephritid is of the genus Ceratitis. In some embodiments, the insect is Ceratitis capitata.

The invention also provides arthropods comprising the gene expression system of the invention. In some embodiments, the arthropod is an insect. In some embodiments, the insect is a Tephritid. In some embodiments, the insect is a Medfly (Ceratitis capitata), a Mexfly (Anastrepha ludens), an Oriental fruit fly (Bactrocera dorsalis), a Spotted-wing drosophila (Drosophila suzukii), an Olive fruit fly (Bactrocera oleae), a Melon fly (Bactrocera cucurbitae), a Natal fruit fly (Ceratitis rosa), a Cherry fruit fly (Rhagoletis cerasi), a Queensland fruit fly (Bactrocera tyroni), a Peach fruit fly (Bactrocera zonata), a Caribbean fruit fly (Anastrepha suspensa) or a West Indian fruit fly (Anastrepha obliqua). In certain embodiments, the Tephritid is of the genus Ceratitis. In some specific embodiments, the insect is Ceratitis capitata. In some embodiments, the insect is female. In some embodiments, the insect is male.

The invention also provides a method of suppressing a wild population of an arthropod comprising breeding a stock of male arthropods comprising the gene expression system of the invention and distributing said stock of male arthropods at a locus of a population of wild arthropods of the same species to be suppressed, whereby matings between said stock male arthropods and said wild arthropods are non-productive due to a detrimental effect on the sperm cells of said male arthropods, thereby suppressing said wild population.

In some embodiments, the detrimental effect on said sperm cells of said male arthropods is conditional and occurs by expression of said effector gene, the expression of said effector gene being under the control of a repressible transactivator protein, the said breeding being under permissive conditions in the presence of a substance, the substance being absent from the said natural environment and able to repress said transactivator. During breeding males for release, the rearing is also done in the absence of the chemical ligand to produce sterile males for release. In some embodiments, the substance is a chemical ligand. In some embodiments, the chemical ligand is tetracycline or an analogue thereof.

In some embodiments, the method suppresses an insect population. In some embodiments, the insect is a Tephritid. In some embodiments, the insect is a Medfly (Ceratitis capitata), a Mexfly (Anastrepha ludens), an Oriental fruit fly (Bactrocera dorsalis), a Spotted-wing drosophila (Drosophila suzukii), an Olive fruit fly (Bactrocera oleae), a Melon fly (Bactrocera cucurbitae), a Natal fruit fly (Ceratitis rosa), a Cherry fruit fly (Rhagoletis cerasi), a Queensland fruit fly (Bactrocera tyroni), a Peach fruit fly (Bactrocera zonata), a Caribbean fruit fly (Anastrepha suspensa) or a West Indian fruit fly (Anastrepha obliqua).

The invention also provides a method of rearing sterile male arthropods comprising rearing arthropods comprising the gene expression system of the invention in the absence of a chemical entity that represses the expression system, thereby activating expression of the effector gene and heterologous polynucleotide encoding functional protein of the expression system, producing sterile, male arthropods.

In some embodiments, the sterile, male arthropods are insects. In some embodiments, the sterile male insect is a Tephritid. In some embodiments, the sterile male insect is a Medfly (Ceratitis capitata), a Mexfly (Anastrepha ludens), an Oriental fruit fly (Bactrocera dorsalis), a Spotted-wing drosophila (Drosophila suzukii), an Olive fruit fly (Bactrocera oleae), a Melon fly (Bactrocera cucurbitae), a Natal fruit fly (Ceratitis rosa), a Cherry fruit fly (Rhagoletis cerasi), a Queensland fruit fly (Bactrocera tyroni), a Peach fruit fly (Bactrocera zonata), a Caribbean fruit fly (Anastrepha suspensa) or a West Indian fruit fly (Anastrepha obliqua).

In specific embodiments, the invention provides a Ceratitis gene expression system for controlled expression of an effector gene in a Ceratitis spp. comprising:

(a) a first expression unit comprising:
i. a first promoter that functions in Ceratitis operably linked to a 5′UTR/CDS gene sequence;
ii. an effector gene operably linked to the 5′UTR/CDS;
iii. a 3′UTR operably linked to the effector gene; and
iv. a tetracycline repressible element operably linked to the promoter, wherein transcription of the effector gene is repressed by tetracycline or an analog thereof;
(b) a second expression unit comprising a coding sequence for a transcription factor (such as tTAV or a homolog, for example) operably linked to an upstream regulatory element, the transcription factor being capable of acting on the first promoter of the first expression unit to drive expression of the effector gene,
wherein the upstream regulatory element comprises:
iii. a first promoter/5′UTR comprising a Ceratitis gene promoter operably linked to a corresponding insect gene 5′UTR (e.g., topi, aly or β-tubulin or homologue thereof);
iv. a second promoter/5′UTR operably linked to the first promoter/5′UTR wherein the second promoter/5′UTR is adjacent to a start site for the transcription of the transcription factor coding sequence;
wherein said first promoter/5′UTR and said second promoter/5′UTR together have testes specificity; and wherein the upstream regulatory element drives sufficient expression of the transcription factor such that the transcription factor drives transcription of the effector gene; and
(c) at least one third expression unit comprising:
i. a heterologous polynucleotide encoding a functional protein, the coding sequence of which is defined between a start codon and a stop codon;
ii. a second promoter capable of initiating transcription in Ceratitis operably linked to the heterologous polynucleotide; and
iii. a splice control polynucleotide which, in cooperation with a spliceosome in Ceratitis, is capable of sex-specifically mediating in Ceratitis
(A) a first splicing of an RNA transcript of the heterologous polynucleotide to produce a first spliced mRNA product, which does not have a continuous open reading frame extending from the start codon to the stop codon; and
(B) an alternative splicing of the RNA transcript to yield an alternatively spliced mRNA product which comprises a continuous open reading frame extending from the start codon to the stop codon,
wherein the functional protein has a lethal effect on Ceratitis
wherein said third expression unit is repressible.

In some embodiments, the Ceratitis gene expression system is an inducible system, where induction or repression occurs by provision or absence of a chemical entity, such as, but not limited to tetracycline or an analogue thereof.

In some embodiments of the Ceratitis gene expression system, the first promoter is a minimal promoter. In some embodiments, the first promoter is an HSP70 minipro promoter, a mini p35 promoter, a mini CMV promoter (CMVm), an Ac5 promoter, a polyhedron promoter, or a UAS promoter.

In some embodiments of the Ceratitis gene expression system, the 5′UTR/CDS is testes-specific. In some embodiments, the 5′UTR/CDS gene sequence is a protamine 5′UTR/CDS or 5′ Protamine B gene sequence. In some embodiments, the 5′UTR/CDS is a Ceratitis capitate Protamine 5′UTR/CDS or a Drosophila melanogaster Protamine B 5′UTR/CDS. In some embodiments, the 5′UTR/CDS gene sequence comprises a polynucleotide sequence that is 80%, 85%, 90%, 95%, 98% or 100% identical to SEQ ID NO:41, SEQ ID NO:69 or SEQ ID NO:93.

In some embodiments of the Ceratitis gene expression system, the 3′UTR is testes-specific. In some embodiments, the 3′UTR is from the same gene as the 5′UTR/CDS gene sequence. In some embodiments, the 3′UTR is a protamine or protamine-like 3′UTR. In certain embodiments, the 3′UTR comprises a polynucleotide sequence that is 80%, 85%, 90%, 95%, 98% or 100% identical to SEQ ID NO:52 or SEQ ID NO:48.

In some embodiments of the Ceratitis gene expression system, the effector gene encodes a nuclease or an interfering RNA. In some embodiments, nuclease is a 3-Zn finger nuclease. In certain embodiments, the 3-Zn finger nuclease is a FokI nuclease. In some embodiments, the FokI nuclease is the endonuclease domain of FokI without a DNA-binding domain. In certain embodiments, the FokI nuclease has a polypeptide sequence that is 80%, 85%, 90%, 95%, 98% or 100% identical to SEQ ID NO:101.

In some embodiments of the Ceratitis gene expression system, the first promoter/5′UTR comprises a topi, aly or β-tubulin promoter or homologue thereof, operably linked to a corresponding topi, aly or β-tubulin 5′UTR.

In some embodiments of the Ceratitis gene expression system, the transcription factor is a heterologous transcriptional activator. In some embodiments, the transcription factor in the second expression unit is tTA or a variant thereof. In some embodiments, tTA is tTAV, tTAV2, or tTAV3.

In some embodiments of the Ceratitis gene expression system, the transcription factor of the second expression unit is tTA or a variant thereof, and the first expression unit comprises a tet operator (tetO).

In some embodiments of the Ceratitis gene expression system, the functional protein is an apoptosis-inducing factor, Hid, Reaper (Rpr), or NipplDm.

In some embodiments of the Ceratitis gene expression system, the RNA transcript comprises two or more coding exons for the functional protein. In some embodiments, the functional protein is conditionally suppressible.

In some embodiments of the Ceratitis gene expression system, the third expression unit comprises at least one positive feedback mechanism, having at least one functional protein to be differentially expressed, via alternative splicing, and at least one promoter therefor, wherein a product of a gene to be expressed serves as a positive transcriptional control factor for the at least one promoter therefor, and whereby the expression of said product is suppressible.

In some embodiments of the Ceratitis gene expression system, an enhancer is associated with the second promoter, and the functional protein enhances activity of said second promoter via the enhancer. In some embodiments, one or more tetO operator units are operably linked with the promoter and act as the enhancer. The tTA or its analogue serves to enhance activity of the promoter via tetO.

In some embodiments of the Ceratitis gene expression system, the functional protein itself a transcriptional transactivator, such as the tTAV system, (e.g., tTAV, tTAV2 or tTAV3).

In some embodiments of the Ceratitis gene expression system, the first expression unit is activated by the presence or absence of a chemical entity. In some embodiments, the second expression unit is activated by the presence or absence of a chemical entity. In some embodiments, the third expression unit is activated by the presence or absence of a chemical entity. In some embodiments, a plurality of expression units are activated by the presence or absence of a chemical entity (e.g., first and second; second and third; first and third; first, second and third). In some embodiments, the chemical entity is tetracycline or an analog thereof.

In some embodiments of the Ceratitis gene expression system, the second promoter is a srya embryo-specific promoter, or a homologue thereof, a Drosophila Hsp70 (e.g., a DmHsp70) promoter or a homologue thereof, or a Drosophila slow as molasses (slam) promoter or a homologue thereof.

In some embodiments of the Ceratitis gene expression system, the splice control polynucleotide is derived from the Ceratitis capitata transformer gene (Cctra), the Drosophila transformer gene (e.g., from D. melanogaster (Dmtra); D. suzukii (Dstra); etc.), the Ceratitis rosa transformer gene (Crtra), or the Bactrocera zonata transformer gene (Bztra). In some embodiments, the splice control polynucleotide is derived from a Drosophila spp. doublesex (dsx) gene, Bombyx mori dsx gene, Pink Boll Worm dsx gene, Ceratitis capitata dsx gene, Codling Moth dsx gene, or a mosquito dsx gene, such as from an Aedes spp. (e.g., Aedes gambiae, Aedes aegypti, etc.).

In some embodiments of the Ceratitis gene expression system, at least one splice control polynucleotide comprises an intron and wherein said intron comprises on its 5′ end, a guanine (G) nucleotide, in RNA. In some embodiments, the splice control polynucleotide comprises an intron and wherein said intron comprises on its 5′ end, UG nucleotides, and UT at its 3′ end, in RNA. In some embodiments, the system comprises a consensus core sequence of WWCRAT, where W=A or T, and R=A or G.

The invention provides plasmids for making genetically engineered Tephritid insects. In specific embodiments, these comprise pOX5257 (SEQ ID NO:95), and pOX5242 (SEQ ID NO:94).

The invention also provides methods of rearing populations of sterile, male insects (e.g., Ceratitis spp. such as Ceratitis capitata), by raising a genetically engineered insect (e.g., C. capitata) comprising an expression system of the invention in the absence of a chemical entity that represses the expression system, thereby activating expression of the effector gene and heterologous polynucleotide encoding functional protein of the expression system, producing sterile, male insects.

The invention also provides methods of suppressing populations of wild-type Ceratitis spp. (e.g., Ceratitis capitata), by releasing genetically engineered male Ceratitis comprising an expression system of the invention, among a population of wild-type Ceratitis, whereupon the genetically engineered Ceratitis males mate with the wild-type female Ceratitis. However, as the male Ceratitis that are released are sterile, the matings are non-productive and no offspring result from such matings, thereby suppressing the population of wild-type Ceratitis.

The invention also provides methods reducing, inhibiting or eliminating crop damage from Ceratitis spp. (e.g., Ceratitis capitata) comprising releasing genetically engineered male Ceratitis comprising an expression system of the invention, among a population of wild-type Ceratitis, whereupon the genetically engineered male Ceratitis mate with the wild-type female Ceratitis. However, as the male Ceratitis that are released are sterile, the matings are nonproductive and no offspring result from such matings, thereby suppressing the population of wildtype Ceratitis and reducing, inhibiting or eliminating crop damage caused by the wild Ceratitis.

### BRIEF DESCRIPTION OF THE DRAWINGS

FIG. 1 shows a schematic diagram of the pOX3864 plasmid regions incorporated into the medfly genome in OX3864A. Four genes (DsRed2, Cctra-tTAV, Bztra-tTAV, VP16) are present in the OX3864A rDNA construct. Due to the two splice modules (Cctra-tTAV and BztratTAV), the tTAV protein is only expressed in females in the absence of tetracycline. Bztra: transformer gene from Bactrocera zonata; Cctra: transformer gene from Ceratitis capitata; DmHsp70 promoter, heat shock protein 70 gene promoter from Drosophila melanogaster; HR5, homologous region 5 enhancer from Autographa californica nucleopolyhedrovirus; IE1 promoter, immediately early gene 1 promoter from Autographa californica nucleopolyhedrovirus; K10 3′-UTR, fs(1) K10 gene 3′-untranslated region; Sry-a, serendipity alpha gene promoter from D. melanogaster; SV40 3′-UTR, Simian virus 40 3′-untranslated region; tTAV, tetracycline transactivator; UTR, untranslated region; VP16, Herpes Simplex Virus 1 Virion Protein 16.

FIG. 2 shows a schematic of the OX3864A rDNA expression vector. Bztra Splicing Module, splice control elements from Bactrocera zonata; Cctra Splicing Module, splice control elements from Ceratitis capitata; DmHsp70 minipro, heat shock protein 70 promoter from Drosophila melanogaster; HR5, homologous region 5 enhancer from Autographa californica nucleopolyhedrovirus; IE1 promoter, immediately early gene 1 promoter from Autographa californica nucleopolyhedrovirus; fs(1) K10 3′-UTR; Sry-a, serendipity alpha gene promoter from D. melanogaster; SV40 3′-UTR, Simian virus 40 3′-untranslated region; tTAV, tetracycline transactivator; UTR, untranslated region; VP16, Herpes Simplex Virus 1 Virion Protein 16.

FIG. 3 shows the splicing patterns for male and female transcripts from the Cctra and Bztra Splicing modules on the OX3864A expression construct. Exons and introns are numbered; octagons represent stop codons. Only female-specific splicing leads to expression of functional tTAV.

FIG. 4 shows a schematic diagram of the OX5257 plasmid.

FIG. 5 shows a schematic diagram of the OX5242 plasmid.

FIG. 6 shows schematic diagrams of the OX5242 and OX5257 rDNA constructs. Four genes are present in each construct; OX5242 and OX5257 differ only in the sperm nuclease gene. Both constructs contain tTAV (expressed only in the male germline through the use of the CcB2Tub2 promoter/5′UTR and the CcHsp83 minipromoter/5′UTR and the CcHsp83 3′ UTR, from C. capitata. Both also contain the DsRed2 transformation marker, which utilises the Mexfly muscle actin (MexMAct) promoter (from Anastrepha ludens) and 3′ UTR to drive strong somatic expression of DsRed2. Both contain the ZsGreen1 sperm marker, which utilises the CcProt promoter, 5′UTR, coding sequence (fused to ZsGreen1) and 3′ UTR, from C. capitata, to drive sperm-specific expression. The OX5242 sperm nuclease uses the D. melanogaster DmHsp70 minipromoter together with the tetracycline operator (tetO x21) to drive expression of the sperm nuclease, which is a fusion protein consisting of protamine from medfly (CcProt) and the endonuclease domain from FokI (derived from Flavobacterium okeanokoites). The OX5257 sperm nuclease uses the D. melanogaster DmHsp70 minipromoter together with the tetracycline operator (tetO x21) to drive expression of the sperm nuclease, which is a fusion protein consisting of protamine from D. melanogaster (DmProtB) and the endonuclease domain from FokI (derived from F. okeanokoites).

FIG. 7 shows a detailed schematic diagram of the OX5257 nuclease cassette showing exons and introns of D. melanogaster ProtB which are spliced to form an open reading frame with FokI endonuclease domain under permissive conditions. Expression is repressible with tetracycline.

### DETAILED DESCRIPTION OF THE INVENTION

### Definitions

This description contains citations to various journal articles, patent applications and patents. These are herein incorporated by reference as if each was set forth herein in its entirety.

The term “penetrance,” as used herein, refers to the proportion of individuals carrying a particular variant of a gene that also express the phenotypic trait associated with that variant. Thus, “penetrance”, in relation to the present invention, refers to the proportion of transformed organisms which express the lethal or sterile phenotype.

The term “construct,” as used herein, refers to an artificially constructed segment of DNA for insertion into a host organism, for genetically modifying the host organism. At least a portion of the construct is inserted into the host organism's genome and alters the phenotype of the host organism. The construct may form part of a vector or be the vector.

The term “transgene,” as used herein, refers to the polynucleotide sequence comprising a first and a second gene expression system to be inserted into a host organism's genome, to alter the host organism's phenotype. The portion of the plasmid vector containing the genes to be expressed is referred to herein as the transfer DNA or recombinant DNA (rDNA).

The term “gene expression system,” as used herein, refers to a gene to be expressed together with any genes and DNA sequences which are required for expression of said gene to be expressed.

The term “splice control sequence,” as used herein, refers to a DNA sequence associated with a gene, wherein the DNA sequence, together with a spliceosome, mediates alternative splicing of a RNA product of said gene. It is believed that the splice control sequence, together with the spliceosome, mediates splicing of a RNA transcript of the associated gene to produce an mRNA coding for a functional protein and mediates alternative splicing of said RNA transcript to produce at least one alternative mRNA coding for a non-functional protein. A “splice control module” may contain multiple splice control sequences that join multiple exons to form a polypeptide encoding nucleic acid.

The term “transactivation activity,” as used herein, refers to the activity of an activating transcription factor, which results in an increased expression of a gene. The activating transcription factor may bind a promoter or operator operably linked to said gene, thereby activating the promoter and, consequently, enhancing the expression of said gene. Alternatively, the activating transcription factor may bind an enhancer associated with said promoter, thereby promoting the activity of said promoter via said enhancer.

The term “lethal gene,” as used herein, refers to a gene whose expression product has a lethal effect, in sufficient quantity, on the organism within which the lethal gene is expressed.

The term “lethal effect,” as used herein, refers to a deleterious or sterilising effect, such as an effect capable of killing the organism per se or its offspring, or capable of reducing or destroying the function of certain tissues thereof, such as reproductive tissues, for example, so that the organism or its offspring are sterile. Therefore, some lethal effects, such as poisons, will kill the organism or tissue in a short time-frame relative to their life-span, whilst others may simply reduce the organism's ability to function, for instance reproductively.

The term “tTAV gene variant,” as used herein, refers to a polynucleotide encoding the functional tTA protein but which differ in the sequence of nucleotides. These nucleotides may encode different tTA protein sequences, such as, for example, tTAV2, tTAV3 and tTAF3.

The term “promoter,” as used herein, refers to a DNA sequence, generally directly upstream to the coding sequence, required for basal and/or regulated transcription of a gene. In particular, a promoter is sufficient to allow initiation of transcription, generally having a transcription initiation start site and a binding site for the RNA polymerase transcription complex.

The term “minimal promoter,” as used herein, refers to a promoter as defined above, generally having a transcription initiation start site and a binding site for the polymerase complex, and further generally having sufficient additional sequence to permit these two to be effective. Other sequences, such as that which determines tissue specificity, for example, may be lacking.

The term “exogenous control factor,” as used herein, refers to a substance which is not found naturally in the host organism and which is not found in a host organism's natural habitat, or an environmental condition not found in a host organism's natural habitat. Thus, the presence of the exogenous control factor is controlled by the manipulator of a transformed host organism in order to control expression of the gene expression system.

The term “tetO element,” as used herein, refers to one or more tetO operator units positioned in series. The term, for example, “tetOx(number),” as used herein, refers to a tetO element consisting of the indicated number of tetO operator units. Thus, references to “tetOx7” indicate a tetO element consisting of seven tetO operator units. Similarly, references to “tetOx14” refer to a tetO element consisting of 14 tetO operator units, and so on.

Where reference to a particular nucleotide or protein sequence is made, it will be understood that this includes reference to any mutant or variant thereof, having substantially equivalent biological activity thereto. In certain embodiments, the mutant or variant has at least 80%, 85%, 90%, 95%, 99%, or 99.9% sequence identity with the reference sequences.

However, it will be understood that despite the above sequence homology, certain elements, in particular the flanking nucleotides and splice branch site must be retained, for efficient functioning of the system. In other words, whilst portions may be deleted or otherwise altered, alternative splicing functionality or activity, to at least 30%, preferably 50%, preferably 70%, more preferably 90%, and most preferably 95% compared to the wild type should be retained. This could be increased compared to the wild type, as well, by suitably engineering the sites that bind alternative splicing factors or interact with the spliceosome, for instance.

As used herein, “splice control module” means a polynucleotide construct in that is incorporated into a vector that, when introduced into an insect, undergoes differential splicing (e.g., stage-specific, sex-specific, tissue-specific, germline-specific, etc.) and thus, for example, creates a different transcript in females than males if the splice control module confers differential splicing in a sex-specific manner.

As used herein, “5′UTR,” refers to an untranslated region of an RNA transcript that is 5′ of the translated portion of the transcript and often contains a promoter sequence.

As used herein, “3′UTR,” refers to an untranslated region of an RNA transcript that is 3′ of the translated portion of the transcript and often contains a polyadenylation sequence.

As used herein, “effector gene” is a gene that when expressed encodes an RNA or protein that has a lethal effect on the organism.

The invention provides plasmids, expression constructs and Mediterranean fruit flies (Medflies) that have elements for sex-specific expression of a lethal gene that results in the death of one sex of Ceratitis spp. The plasmids, constructs and Medflies containing such expression constructs also have elements for testes-specific expression of an effector gene, that when expressed, is detrimental to sperm development, rending the males sterile. The elements are repressible, such as by a chemical entity (e.g., tetracycline or an analog thereof). The constructs to impart sex-selection and male sterility may be found on a single plasmid or expression construct or may be on different plasmids or expression constructs. In particular, the invention relates to Medflies transformed with these constructs, particularly Ceratitis capitata.

The expression system of the invention comprises three particular features: (1) an expression unit that provides alternative splicing of a transcript that leads to expression of a gene in one sex that is lethal, but does not lead to expression in the other sex (allowing sex selection of the insects); (2) an expression unit that provides a positive feedback mechanism to promote transcription of a transcription factor to drive expression of the transcription factor to act on each expression unit; and (3) an expression unit that confers sterility to males.

### 1. Sex Selection

i. Splice Control Modules

The present invention provides a splice control module polynucleotide sequence which provides for differential splicing (such as, for example, sex-specific, stage-specific, germline-specific, or tissue-specific splicing) in an insect. In particular, the invention provides a splice control module which provides for sufficient female-specificity of the expression of a gene of interest. In certain embodiments of the invention, the gene of interest is a gene that imparts a lethal effect. For convenience, the description will refer to a lethal effect, however, it will be understood that the splice module may be used on other genes of interest as described in further detail below. In the specific embodiments, the splicing module provides female-specific splicing to allow expression of a lethal gene in the female insect such that under conditions in which transcription and splicing occurs (e.g., in the absence of tetracycline) only females produce the lethal protein and die while male insects survive, but pass on the lethal gene to their offspring. The splice control module allows an additional level of control of protein expression, in addition to the promoter.

The gene of the splice control module comprises a coding sequence for a protein or polypeptide, i.e., at least two exons, and in some embodiments, for example, three or more exons, capable of encoding a polypeptide, such as a protein or fragment thereof. An “exon” in this context could also simply be a start codon. In certain embodiments, the different exons are differentially spliced together to provide alternative mRNAs. In certain embodiments, the alternative spliced mRNAs have different coding potential, i.e., encode different proteins or polypeptide sequences. Thus, the expression of the coding sequence is regulated by alternative splicing.

Each splice control module in the system comprises at least one splice acceptor site and at least one splice donor site. The number of donor and acceptor sites may vary, depending on the number of segments of sequence that are to be spliced together.

In some embodiments, the splice control module regulates the alternative splicing by means of both intronic and exonic nucleotides. It will be understood that in alternative splicing, sequences may be intronic under some circumstances (i.e., in some alternative splicing variants where introns are spliced out), but exonic under other. In other embodiments, the splice control module is an intronic splice control module. In other words, the splice control sequence is substantially derived from polynucleotides that form part of an intron and are thus excised from the primary transcript by splicing, such that these nucleotides are not retained in the mature mRNA sequence.

As mentioned above, exonic sequences may be involved in the mediation of the control of alternative splicing, but it is preferred that at least some intronic control sequences are involved in the mediation of the alternative splicing.

The splice control module may be removed from the pre-mRNA, by splicing or retained so as to encode a fusion protein of at least a portion of the gene of interest to be differentially expressed. In some embodiments, the splice control module does not result in a frameshift in the splice variant produced. In some embodiments, this is a splice variant encoding a full-length functional protein.

Interaction of the splice control module with cellular splicing machinery, e.g., the spliceosome, leads to or mediates the removal of a series of, for example, at least 20, 30, 40 or 50 consecutive nucleotides from the primary transcript and ligation (splicing) together of nucleotide sequences that were not consecutive in the primary transcript (because they, or their complement if the antisense sequence is considered, were not consecutive in the original template sequence from which the primary transcript was transcribed). The series of at least 50 consecutive nucleotides comprises an intron. In some embodiments, this mediation acts in a sex-specific manner. In some embodiments, it is female-specific such that equivalent primary transcripts in different sexes, and optionally also in different stages, tissue types, etc., tend to remove introns of different size or sequence, or in some cases may remove an intron in one case but not another. This phenomenon, the removal of introns of different size or sequence in different circumstances, or the differential removal of introns of a given size or sequence, in different circumstances, is known as “alternative splicing.” Alternative splicing is a well-known phenomenon in nature, and many instances are known.

In some embodiments in which the mediation of alternative splicing is sex-specific, the splice variant encoding a functional protein to be expressed in an organism is the F1 splice variant, i.e., a splice variant where the F denotes it is found only or predominantly in females, although this is not essential.

When exonic nucleotides are to be removed, then these must be removed in multiples of three (entire codons), if it is desired to avoid a frameshift, but as a single nucleotide or multiples of two (that are not also multiples of three) if it is desired to induce a frameshift. It will be appreciated that if only one or certain multiples of two nucleotides are removed, then this could lead to a completely different protein sequence being encoded at or around the splice junction of the mRNA.

This is particularly the case in an embodiment of the system where cassette exons are used to interrupt an open reading frame in some splice variants but not others, such as in, for example, tra, especially Cctra (see below).

Correspondingly, for configurations where all or part of a functional open reading frame is on a cassette exon, this cassette exon may be included in transcripts found only or predominantly in females, and preferably such transcripts are, individually or in combination, the most abundant variants found in females, although this is not essential.

In one embodiment, sequences are included in a hybrid or recombinant sequence or construct which are derived from naturally occurring intronic sequences which are themselves subject to alternative splicing, in their native or original context. Therefore, an intronic sequence may be considered as one that forms part of an intron in at least one alternative splicing variant of the natural analogue. Thus, sequences corresponding to single contiguous stretches of naturally occurring intronic sequence are envisioned, but also hybrids of such sequences, including hybrids from two different naturally occurring intronic sequences, and also sequences with deletions or insertions relative to single contiguous stretches of naturally occurring intronic sequence, and hybrids thereof. Said sequences derived from naturally occurring intronic sequences may themselves be associated, in the invention, with sequences not themselves part of any naturally occurring intron. If such sequences are transcribed, and preferably retained in the mature RNA in at least one splice variant, they may then be considered exonic.

It will also be appreciated that reference to a “frame shift” could also refer to the direct coding of a stop codon, which is also likely to lead to a non-functioning protein as would a disruption of the spliced mRNA sequence caused by insertion or deletion of nucleotides. Production from different splice variants of two or more different proteins or polypeptide sequences of differential function is also envisioned, in addition to the production of two or more different proteins or polypeptide sequences of which one or more has no predicted or discernable function. Also envisioned is the production from different splice variants of two or more different proteins or polypeptide sequences of similar function, but differing subcellular location, stability or capacity to bind to or associate with other proteins or nucleic acids.

Some embodiments of the invention include a modified tra, sxl or dsx intron. One may choose to delete, as we have done in the Examples, sizable amounts from alternatively spliced introns whilst still retaining the alternative splicing function. Thus, whilst large deletions are envisioned, it is also envisaged that smaller, e.g., even single nucleotide insertions, substitutions or deletions may be used.

a. Splice module Transformer (Tra)

In some embodiments, at least one splice control module is derived from a tra intron. The Ceratitis capitata tra intron from the transformer gene was initially characterised by Pane et al. (2002) Development 129:3715-3725. In insects, for instance, the tra protein is differentially expressed in different sexes. In particular, the tra protein is known to be present largely in females and, therefore, mediates alternative splicing in such a way that a coding sequence is expressed in a sex-specific manner, i.e., that in some cases a protein is expressed only in females or at a much higher level in females than in males or, alternatively, in other cases a protein is expressed only in males, or at a much higher level in males than in females. The mechanism for achieving this sex-specific alternative splicing mediated by the tra protein or the TRA/TRA-2 complex is known and is discussed, for instance, in Pane et al. (2002) Development 129:3715-3725.

It will be appreciated that homologues of the Ceratitis capitata tra intron from the transformer gene exist in other species, and these can be easily identified in said species and also in their various genera. Thus, when reference is made to tra it will be appreciated that this also relates to tra homologues in other species. Thus, in some embodiments each of the alternative splicing mechanisms is independently derived from a tra intron of a C. capitata ortholog or homolog. In some embodiments, the ortholog or homologue is from an arthropod, such as an insect of the order Diptera, such as a tephritid. In other embodiments, the ortholog or homologue is from the genus Cochliomyia, Glossina, Lucilia, Musca, Ceratitis, Bactrocera, Anastrepha or Rhagoletis. In other embodiments, the ortholog or homolog is from Ceratitis rosa, or Bactrocera zonata. In further embodiments, the ortholog or homolog is from a Drosophilid, such as, but not limited to Drosophila americana, Drosophila erecta, Drosophila hydei, Drosophila mauritania, Drosophila melanogaster, Drosophila sechellia, Drosophila simulans, Drosophila suzukii, and Drosophila virilis. In further embodiments, the ortholog or homolog is from Bactrocera zonata, B. tryoni, B. cucurbitae or B. oleae and this ortholog or homolog is referred to herein as Bztra (GenBank accession number BzTra KJ397268). Orthologs may also be from the Order Hymenoptera, or Coleoptera. Examples, include, but are not limited to Apis cerana, Apis dorsata, Apis florea, Apis mellifera, Atta cephalotes, Bombus impatiens, Bombus terrestris, Camponotus floridanus, Euglossa hemichlora, Harpegnathos saltator, Linepithema humile, Melipona compressipes, Megachile rotundata, Nasonia giraulti, Nasonia longicornis, Nasonia vitripennis, Pogonomyrmex barbatus, Solenopsis invicta, and Tribolium castaneum.

The splicing pattern among tra genes in particular is well conserved, with those transcripts found in males containing additional exonic material relative to the F1 transcript, such that these transcripts do not encode full-length, functional tra protein. By contrast, the F1 transcript may encode full-length, functional TRA protein; this transcript is substantially female-specific at most life-cycle stages, though it is speculated that very early embryos of both sexes may contain a small amount of this transcript. In other embodiments, a truncated tra is used with fewer tra exons and introns. In each case, the sequence is spliced out of the Cctra F1 transcript, but not the male-specific or non-sex-specific transcripts, as the tra intron, or even the tra F1 intron. In other embodiments, the tra gene is derived from B. zonata (Bztra). For clarity, the tra intron is a general term, but a specific tra intron derived from a particular species will be referred to by the species designation: e.g., Ceratitis capitata (Cctra intron), B. zonata, (Bztra intron), etc.

Thus the tra gene is regulated in part by sex-specific alternative splicing, while its key product, the tra protein, is itself involved in alternative splicing. In insects, sex-specific alternative splicing mediated by the TRA protein, or a complex comprising the TRA and TRA2 proteins, include Dipteran splice control sequences derived from the doublesex (dsx) gene and also the tra intron itself, although this would exclude the tra intron from Drosophila (Dmtra), which is principally mediated by the Sxl gene product in Drosophila, rather than tra or the TRA/TRA2 complex.

By “derived” it will be understood that, using reference to the tra intron, this refers to sequences that approximate to or replicate exactly the tra intron, as described in the art, in this case by Pane et al. (2002), supra. However, it will be appreciated that, as these are intronic sequences, that some nucleotides can be added or deleted or substituted without a substantial loss in function.

If more than one splice control module is incorporated into a gene expression system of the invention, the splice control module may be the same or different. In some embodiments, the splice control modules are derived from different species in order to reduce the risk of recombination. Thus, in some embodiments, one of the first and second splice control sequences is Cctra and the other is derived from a different species. In one embodiment, one of the first and second splice control sequences is Cctra and the other is Bztra (GenBank accession number BzTra KJ397268).

In a particular embodiment, the first splice control sequence is Cctra and the second splice control sequence is Bztra (GenBank accession number BzTra KJ397268). The exact length of the splice control sequence derived from the tra intron is not essential, provided that it is capable of mediating alternative splicing. In this regard, it is thought that around 55 to 60 nucleotides is the minimum length for a modified tra intron, although the wild type tra intron (F1 splice variant) from C. capitata is in the region of 1345 nucleotides long. In some embodiments, the splice control module has a sequence of SEQ ID NO:7. In other embodiments the Splice control module has at least one intron that is spliced which may be Intron 1 or Intron 2 of Cctra or truncations or derivatives thereof. In some embodiments, the Splice control module has an Intron 1 of SEQ ID NO:10 and/or an Intron 2 of SEQ ID NO:13. In other embodiments, the Splice control module further comprises (in addition to one or more introns) at least two exons selected from Exon 1a, Exon 1b, Exon 2a, and Exon 2b, or derivatives or truncations thereof. In other embodiments, the Splice control module comprises the following elements: an Exon 1a of SEQ ID NO:8, an Exon 1b of SEQ ID NO:9, an Intron 1 of SEQ ID NO:10, an Exon 2a of SEQ ID NO:11, an Exon 2b of SEQ ID NO:12, and an Intron 2 of SEQ ID NO:13.

In a particular embodiment, the second splice control module is derived from a Bztra and comprises the sequence of SEQ ID NO:18. In other embodiments the Splice control module has at least one intron that is spliced which may be Intron 1 or Intron 2 of Bztra or truncations or derivatives thereof. In some embodiments, the Splice control module has an Intron 1 of SEQ ID NO:20 and/or an Intron 2 of SEQ ID NO:22. In other embodiments, the Splice control module further comprises (in addition to one or more introns) Bztra Exon 1 and Exon 2 or truncations or derivatives thereof. In other embodiments, the Splice control module comprises the following elements: an Exon 1 of SEQ ID NO:19, an Intron 1 of SEQ ID NO:20, an Exon 2 of SEQ ID NO:21, and an Intron 2 of SEQ ID NO: 22.

b. Splice Module Doublesex (Dsx)

The splice module may also be derived from the doublesex (dsx) gene. The dsx gene may be derived from insect species such as from Dipteran or Lepidopteran insects, including but not limited to Drosophila, Bombyx, Pectinophora, Cydia, Bactrocera, Ceratitis, and mosquitoes such as Anopheles and Aedes. In some embodiments, the dsx gene is derived from Ceratitis capitata, Drosophila melanogaster, Bactrocera zonata, Bactrocera oleae, Bactrocera tyroni, Bombyx mori, Pectinophora gossypiella, Cydia pomonella, Anopheles gambiae or Aedes aegypti.

Various forms of dsx splice modules may be used in the invention. A Bombyx mori dsx mini-gene construct (containing exonic sequence and truncated intronic sequence) has been transformed into B. mori and the germline transformants show sex-specific splicing (Funaguma, S. et al., (2005) J. Insect Sci. 5(17):1-6). In a dsx Splice module based on the B. mori dsx, the minigene may have the first exon deleted as well as the intron between Exons 3 and 4 (Female specific exons). Various splice modules using dsx components derived from various insects are described in U.S. Pat. No. 9,970,025. The splicing of the Aedes aegypti gene appears to be similar to Anopheles gambiae dsx (Scali, C. et al. (2005) J Exp. Biol. 208(19):3701-37009). The Ae. Aegypti dsx male-specific transcript (M1) is produced which does not include exons 5a or 5b. Two female specific splice variants (F1 and F2) have the following structure; F1 comprises exons 1-4, 5a, 6 and 7 but not 5b, F2 comprises exons 1-4 and 5b, and perhaps 6 and 7. In addition, a further transcript (C1) is present in both males and females; this comprises exons 1-4 and 7, but not exons 5a, 5b or 6. Thus, a Splice module derived from Ae. aegypti dsx, the splice modules to produce a female transcript in-frame with a gene of interest. The splice module comprises at least two exons and at least one intron from the dsx gene. In some embodiments, the exons and/or intron(s) are truncated to provide for smaller polynucleotide sequences or better splicing. In some embodiments, the splice control module comprises an exon 4 of dsx; an intron 4 of dsx, or a truncated version thereof comprising a 5′ terminal fragment of the dsx intron 4 and a 3′ fragment of the dsx intron 4; and an exon 5a of dsx. In other embodiments, the Splice module comprises an exon 4 of dsx; an intron 4 of dsx (or a truncated version thereof comprising a 5′ terminal fragment of the dsx intron 4 and a 3′ fragment of the dsx intron 4); an exon 5a of dsx, an intron 5 of dsx (or portion thereof); an exon 5b of dsx (or modified version thereof, a truncated intron 6 of dsx comprising a 5′ terminal fragment of the dsx intron 6 and a 3′ fragment of the dsx intron 6; and a 5′ fragment of exon 6. Splice modules based on Aedes aegypti dsx may be found, for example, in WO 2018/029534.

iii. Splicing

Introns typically consist of the following features (given here as the sense DNA sequence 5′ to 3′); in RNA thymine (T) will be replaced by uracil (U)):
a. 5′ end (known as the splice “donor”): GT (or possibly GC)
b. 3′ end (known as the splice “acceptor”): AG
c. Upstream/5′ of the acceptor (known as the “branch point”): A-polypyrimidine tract, i.e. AYYYYY . . . Yn

The terminal nucleotides of exons immediately adjacent to the 5′ intronic splice “donor” and the 3′ intronic splice “acceptor” are typically G.

In some embodiments, the splice control module is immediately adjacent, in the 3′ direction, to the start codon, so that the G of the ATG is 5′ to the start (5′ end) of the splice control module. This may be advantageous as it allows the G of the ATG start codon to be the 5′ G flanking sequence to the splice control module.

Alternatively, the splice control module is 3′ to the start codon but within 10,000 exonic bp, 9,000 exonic bp, 8,000 exonic bp, 7,000 exonic bp, 6,000 exonic bp, 5,000 exonic bp, 4,000 exonic bp, 3,000 exonic bp, 2000 exonic bp, or 1000 exonic bp, 500 exonic bp, 300 exonic bp, 200 exonic bp, 150 exonic bp, 100 exonic bp, 75 exonic bp, 50 exonic bp, 30 exonic bp, 20 exonic bp, 10 or even 5, 4, 3, 2, or 1 exonic bp.

In some embodiments, branch points are included in each splice control sequence, as described above. A branch point is the sequence to which the splice donor is initially joined which shows that splicing occurs in two stages, in which the 5′ exon is separated and then is joined to the 3′ exon.

The sequences provided can tolerate some sequence variation and still splice correctly. There are a few nucleotides known to be important. These are the ones required for all splicing. In some embodiments, the initial GU and the final AG of the intron are present, as discussed elsewhere, though ˜5% of introns start GC instead. In some embodiments, the consensus sequence is used, although it applies to all splicing, not specifically to alternative splicing.

### 2. Positive Feedback

The expression systems of the invention also comprise a positive feedback mechanism, wherein the product (which may either be RNA or the translation product thereof) acts at a transcription enhancer, normally by binding the enhancer site, and enhancing promoter activity. Enhancement of the promoter activity then serves to increase transcription of the gene for the product which, in turn, further serves to either lift inhibition or enhance promotion, thereby leading to a positive feedback loop.

Control of the product may be by any suitable means, and may be effective at any level. For example, the control may be effective either to block transcription of the control effector gene or to block translation of the RNA product thereof, or to prevent or inhibit action of the translation product of the gene.

For example, the gene product of tTA (tetracycline-repressible transcription activator) acts at the tetO operator sequence (Baron and Bujard, 2000; Gossen et al., 1994; Gossen and Bujard, 1992). Upstream of a promoter, in either orientation, tetO is capable of enhancing levels of transcription from a promoter in close proximity thereto, when bound by the product of the tTA gene. If the tTA gene is part of the cassette comprising the tetO operator together with the promoter, then positive feedback occurs when the tTA gene product is expressed.

Control of this system is readily achieved by exposure to tetracycline, which binds to the gene product and prevents transactivation at tetO.

The tTA system also has the advantage of providing stage-specific toxicity in a number of species. In particular, “squelching” is observed in the development phases of many insects, the precise phase of susceptible insects being species-dependent (Gill, G. and M. Ptashne (1988) Nature 334(6184):721-724). Some insects may reach pupation before the larva dies, while others die early on. Susceptibility ranges from 100% fatality to a small reduction in survival rates. In general, though, adult insects appear to be immune to the squelching effect of tTA, so that it is possible to raise insects comprising a tTA positive feedback system in the presence of tetracycline, and then to release the adult insects into the wild. These insects are at little or no competitive disadvantage to the wild type, and will breed with the wild type insects, but larvae carrying the tTA positive feedback cassette will die before reaching maturity, in the absence of tetracycline or tetracycline analogs.

There are various forms of tTAV and each may be used in the invention provided it acts as a transactivator and binds to the enhancer site to enhance transcription in the gene expression system. Examples of useful tTAVs are tTAV, tTAV2, and tTAV3. Examples of polypeptide sequences of these are SEQ ID NO:88, SEQ ID NO:89, and SEQ ID NO:90, respectively. Examples of polynucleotides encoding these proteins are provided as SEQ ID NO:61, SEQ ID NO:86, and SEQ ID NO:87, respectively.

While, where at least one of the gene expression systems is a positive feedback loop, the activating transcription factor of said positive feedback loop activates the promoter of said gene expression system, in some embodiments the activating transcription factor also activates the promoter of another gene expression system.

In some embodiments, one of the gene expression systems is a linear gene expression system, and the other is a positive feedback loop, as described above.

In some embodiments, there are two or more gene expression systems that act as positive feedback loops. Each of the first and second gene expression systems expresses a different lethal gene product, such that the lethal gene product of the first gene expression system acts as the activating transcription factor for only the first gene expression system, and vice versa. Such expression systems are described in more detail in US2017/0188559 (WO 2015/185933).

In some embodiments, both the first and the second gene expression systems act as positive feedback loops and express the same or similar lethal products. Thus, the lethal gene product expressed by the first gene expression system acts as an activating transcription factor for both the first and the second gene expression system, and vice versa. Accordingly, in some embodiments, both the first and the second gene expression systems comprise tTA or a tTAV gene variant as both the lethal gene and the gene encoding the activating transcription factor. Accordingly, both gene expression systems comprise an enhancer which is a tetO element as described above, which drives expression from the associated promoter. The first activating transcription factor (i.e. the first lethal gene product) can bind both the first and the second enhancers, and the second activating transcription factor can bind both the first and the second enhancers.

### 3. Male Sterility

The expression system of the invention also includes an insect male germline expression unit for use in combination with the sex selection and positive feedback expression units suitable for conditional expression of an effector gene in an insect male germline.

The sterility expression unit comprises an effector gene and a promoter therefor operably linked thereto in which the promoter may be acted upon by a transcription factor to drive expression of the effector gene before or during meiosis. Without wishing to be bound by any particular theory of operability, it is believed that the effector gene is transcribed before meiosis and translated after, however, it is also possible that transcription and translation may occur during or after meiosis. It is within the scope of the invention that the effector gene is transcribed such that it is translated into the effector protein in a way that permits sperm production but prevents effective fertilization of the egg by causing DNA damage to the sperm DNA. The expression unit also contains an upstream regulatory element including: a promoter for the transcription factor; and a 5′UTR adjacent a translation start site for the transcription factor coding sequence; the upstream regulatory element driving sufficient expression of the transcription factor such that the transcription factor protein in turn drives transcription of the effector gene before or during meiosis. The unit also contains a repressible element operably linked to the promoters linked to the effector gene and transcription promoter, wherein transcription of both the transcription factor and the effector gene is repressed, for example, by addition of a chemical ligand (e.g., tetracycline or a tetracycline analog).

In some embodiments, the transcription factor is a transcriptional activator, such as tTA, GAL4 or their variants. The effector may be, for example but not by way of limitation, an endonuclease (e.g., a 3-Zn finger nuclease, a restriction endonuclease, etc.) (described in more detail below).

In some embodiments, the sterile expression unit results in sterilization allowing the organism to compete in the natural environment (“in the wild”) with wild-type organisms, but the sterile organism cannot then produce viable offspring. In this way, the present invention achieves a similar or better result to techniques such as the Sterile Insect Technique (SIT) in insects, without the problems associated with SIT, such as the cost, danger to the user, and reduced competitiveness of the irradiated organism.

The promoter of the sterility expression unit that is operably linked to the effector gene should have a germline effect and in some embodiments, expression of the system is conditional. Ideally, spermatogenesis should be substantially completed before any negative effects of the expression of the effector are seen. It is preferred that there is no discernable effect on sperm function until after egg entry. While DNA damage could perhaps be seen as “a negative effect,” one can view DNA in a sperm merely as “cargo” as there is no transcription in the sperm. Any DNA damage caused by the effector must be sufficient to prevent the production of viable progeny. Thus, the present invention provides conditional germline specificity (in terms of expression).

In respect of the regulatory elements, particularly the promoter and/or 5′UTR (sometimes referred to herein as the “promoter/5′UTR”) of the upstream regulatory element, it is desirable that there is no delayed translational effect for the present transcription factor. One way to achieve this is to use the regulatory elements from a gene known to transcribe and translate at a sufficient, and in some embodiments, strong, level before meiosis. Suitable examples would include chaperone genes, such as, but not limited to the HSP family of genes, in particular hsp83. In another embodiment, the 3′UTR may be derived from a virus, such as SV40.

In some embodiments, the sterility expression unit comprises a promoter from Beta 2 tubulin (B2T) combined with a modified B2T 5′UTR or a 5′UTR from Hsp83. Optionally, a 3′UTR from SV40 may be used. Either or both of the promoter and the 5′UTR may be from topi. The term “topi” refers to the Drosophila gene matotopetli. However, the present invention includes functional homologues and paralogues from other species. These can be identified by reference to the conserved open reading frame (ORF) as described above. In the case of a 5′UTR from topi, the promoter may also be from topi, although it is envisaged that it could be from any other of the promoters disclosed herein, for instance B2T. Again, when the promoter from topi is used and/or the 5′UTR from topi is used, the 3′UTR may also be from topi, as are the remaining regulatory elements such as the 5′ cap and the polyA tail. The reason for this is that topi has an “early” expression pattern in spermatogenesis, such that it is able to drive suitable transcription and translation after mitotic divisions but prior to meiosis.

In the case of B2T, while the promoter is useful, the 5′UTR of B2T can be replaced or supplemented by the 5′UTR from, for instance, a chaperone such as Hsp83. Thus, in some embodiments, the promoter and regulatory elements are homologous to each other, in other embodiments, the promoters and regulatory elements are heterologous to each other. In some embodiments both the promoter and 5′UTR are from B2T In other embodiments, both the promoter and 5′UTR are from Hsp83.

In another aspect, the present invention also provides an arthropod, transformed with the present system or by the present methods. In other words, the invention also provides a transformant or a genetically modified arthropod, as further defined herein. It will be appreciated that in some embodiments, the arthropod is a male, preferably whose gonads carry the present system, such that expression of the effector occurs during spermatogenesis.

It is an advantage of the present invention that the promoter and the regulatory elements act together in synergy to provide the desired expression pattern.

As mentioned above, the promoter may be from a testis-specific gene or at least one sufficient to provide “early” expression during spermatogenesis. Alternatives include promoters in the tubulin family, particularly the beta tubulins such as, for example, the B2T promoter, and homologues thereof. When this is used, it is necessary to use upstream regulatory element that does not have the translational delay signals seen with at least some instances of B2T's upstream regulatory element. An advantage to using the B2T promoter is that the B2T gene coding sequence is highly conserved and it and a suitable promoter fragment can be readily identified and isolated from a given arthropod species by a skilled person.

Examples of a B2T promoter sequence are provided as SEQ ID NO:38 and SEQ ID NO:65. An example of the B2T promoter 5′UTR sequence is provided as SEQ ID NO:64 and SEQ ID NO:37.

If B2T promoters from other species are used in the present invention, then a skilled person will be readily able to identify the 5′UTR based on its conserved nature from the above SEQ ID NO. They will then be able to replace it with another 5′UTR. Examples include the 5′UTR from chaperones, particularly the hsp family, particularly hsp83. A suitable example, the 5′UTR from hsp83 is provided as SEQ ID NO:62 and SEQ ID NO:35.

An example of a 3′UTR that may be used in the gene expression systems of the invention includes that from SV40. An example of the sequence is provided as SEQ ID NO:16.

The topi coding sequence is largely conserved between mosquitoes such as Aedes aegypti and Medfly (C. capitata). As for B2T, one can clone the coding region of topi (or part of it) by sequence similarity (there are many methods of determining sequence similarity including molecular and sequence-based ones as is known to those of skill in the art). Generally the promoter may be found 5′ to the transcription start. It is not always clear how much 5′ sequence will be needed that will contain the promoter, but a conservative approach would be to use all the 5′ DNA before the next transcribed region. However, in practice, male germline promoters tend to be relatively short (a few hundred bases). Thus, one of skill in the art would know to use about 1 kb of DNA 5′ of the transcription start site to comprise the promoter. One of skill in the art would also know how to make and test smaller amounts of the 5′ DNA to narrow the sequence necessary that acts as the promoter.

Topi is useful because it has early expression and is linked to spermatogenesis. It is also advantageous because it is a relatively compact system, i.e., consists of relatively few polynucleotides. It is testes-specific and is expressed earlier than B2T. The expression of topi compared to a B2T promoter is weaker, but this may be advantageous in embodiments for which expression needs to be modified. Topi is an example of a transcription factor and so promoters and/or regulatory elements from other transcription factors that express in the testes and are testes-specific (i.e., expressed only in the testes) are preferred.

A stronger overall sterilisation effect was seen in crosses where nuclease expression was driven by topi promoter, compared to B2-tubulin, particularly in Aedes aegypti. Nevertheless, significant male sterility was observed in both cases, rendering both topi and the altered form of B2-tubulin suitable promoters for the “paternal lethality effect” in mosquitoes, especially Aedes aegypti.

Genes whose product (e.g. encoded protein) is required only at or after meiosis are likely to be translated only shortly before, or after, meiosis, even if transcribed earlier. In contrast, transcription factors needed to drive the expression of such genes must be expressed (transcribed and translated) early enough for their protein product to accumulate sufficiently to drive adequate expression of target genes prior to the cessation of transcription before the meiotic divisions. Therefore, where it is desired to express a transcriptional activator such as tTA in the male germline, the regulatory elements of a male germline transcription factor may be suitable with minimal modification.

Suitable endonucleases are described in greater detail below. However, certain embodiments include zinc-finger endonucleases. Other alternatives include IppO1, also referred to as I-PpoI, as used by Crisanti et al (Catteruccia et al., (2009) Malar J. 8 (Suppl 2):S7; Windbichler et al., (2011) Nature 473(7346):212-215; Windbichler et al., (2007) Nucleic Acids Res. 35(17): 5922-5933; Windbichler et al., (2008) PLoS Genet. 4(12): e1000291. IppO1 has certain advantages such as that it has a very long recognition sequence, which is correspondingly rare in random sequences. However, IppO1 does not have high specificity relative to some restriction enzymes. For example, it will tolerate (i.e. still cut) sequences that have a degree of divergence from canonical recognition sequences.

Windbichler et al. (2008) PLoS Genet. 4(12):e1000291, showed that expression of IPpoI, (which was thought only to cut the X chromosome in An. gambiae) gave completely sterile males, producing no viable female offspring due to damage to the paternally-derived X chromosome, but did produce viable male offspring. Their proposed explanation, for which they provide some supporting data, was that the I-PpoI itself is transmitted in the sperm to the fertilized egg, where it cuts the maternally-derived X chromosome as well.

An alternative endonuclease is the FokI protamine fusion endonuclease. The FokI nuclease may be encoded by a codon-optimized polynucleotide encoding an amino acid sequence of SEQ ID NO:101. As used herein, “FokI nuclease” includes a polypeptide having the FokI endonuclease without a DNA-binding domain. The FokI protein may be at least 80%, 85%, 90%, 95%, 98% identical to the polypeptide of SEQ ID NO:101, provided the protein retains nuclease activity. In some embodiments, the polynucleotide sequence of FokI is 80%, 85%, 90%, 95%, 98% or 100% identical to the polynucleotide sequence of SEQ ID NO:73 or SEQ ID NO:47. Further alternatives include the EcoRI protamine fusion endonuclease. Protamine is a DNA binding protein and generally has very low sequence specificity. Without wishing to be bound by any particular theory of operability, it is believed that this is combined with FokI, to form a cleavage domain. This cleavage domain must dimerise in order to cleave its target, giving rise to non-linear concentration effects. An effector that acts as a monomer is expected to have its effect (i.e., DNA cleavage) in proportion to its concentration. For some embodiments, a non-linear dose-response curve may be advantageous, so that the effect is near zero up to a certain concentration, but increases to full effectiveness above that concentration, the limit of this being a binary threshold effect. The protamine-FokI is an example having a degree of non-linearity.

Protamine binds DNA but has little or no sequence specificity. Therefore, at low concentrations (e.g. molecules per nucleus) the protamine-FokI proteins will tend to be scattered randomly around the chromatin, rarely being in sufficiently close proximity/orientation to dimerise and cut a chromosome. However, as the concentration increases the probability of such proximity greatly increases, leading to a non-linear relationship between concentration and cutting. This facilitates the selection of a promoter (and specific transgene insertion), as the system is relatively inert even with low-but non-zero levels of off-target (basal) expression, while still having the desired effect at higher expression (in the intended expression domain, de-repressed in the case of a repressible expression system). A similar effect can be achieved where the effector must dimerise (or form a larger complex, e.g. tetramer) prior to binding to DNA. Where a more linear effect is desired, this may readily be accomplished within the method of the invention, by using a nuclease domain that does not need to dimerise, or where the necessary subunits are provided in a single polypeptide (e.g. two copies of the FokI domain rather than one). Additional manipulation of the system can be achieved by using nucleases of greater or lesser sequence specificity, as the available protein molecules will be focused by the specificity and affinity of the DNA binding domain to a larger or smaller number of sites, leading to a greater or lesser degree of concentration at those sites.

In some embodiments, the protamine gene (or protein coding sequence) is obtained from the same species as that of the target species. In some embodiments, the protamine gene is derived from D. melanogaster. In some embodiments, the protamine gene is derived from Aedes aegypti.

Other type II endonucleases include but are not limited to Eco32I, BfiI, and MboII. These endonucleases are homodimeric (they only cleave DNA when dimerized) and make double stranded DNA breaks.

Other endonucleases may include, for example, HEG's (Homing Endonucleases) which can be monomers or dimers but generally have low specificity as they tolerate a relatively high level of imperfect matches in their very long recognition sequences, but they certainly don't just cut random sequences). Other alternatives include restriction endonucleases from bacteria, which also have low specificity.

Accordingly, one of skill in the art can select the desired level of specificity for the application. Thus, a further level of fine-tuning is possible by appropriate selection of endonucleases as the effector. The nuclease effector fusion protein has been found to be fully functional in three different Diptera species tested so far, namely C. capitata, B. oleae and Aedes aegypti. These species are useful in the invention as are other species in the same genus.

In some embodiments, the promoter of the promoter/5′UTR is a promoter such as an Hsp70 minipro, a P minimal promoter, a CMV minimal promoter, an Act5C-based minimal promoter, a BmA3 promoter fragment, and an Adh core promoter (Bieschke, E. et al. (1998)Mol. Gen. Genet., 258:571-579) and a 5′UTR derived from a protamine gene (e.g., a protamine or Protamine B gene).

### Heterologous Genes of Interest

The system may also be capable of expressing at least one protein of interest, i.e., said functional protein to be expressed in an organism. Said at least one protein of interest may have a therapeutic effect or may be a marker (for instance AmCyan, Clavularia, ZsGreen, ZsYellow, Discosoma striata, DsRed2, AsRed, Discosoma Green, Discosoma Magenta, HcRed-2A, mCherry, Green Fluorescent Protein (GFP), Red Fluorescent Protein (RFP), and HcRed-Crl-tandem, and the like, or one or more of their mutants or variants), or other markers that are well known in the art such as drug resistance genes. Other proteins of interest may be, for example, proteins that have a deleterious, lethal or sterilizing effect. Alternatively, a gene of interest may encode an RNA molecule that has an inhibitory effect. Further proteins to be expressed in the organism are, or course, envisaged, in combination with said functional protein, such as a lethal gene as discussed below.

In some embodiments, the expression of the heterologous polynucleotide sequence leads to a phenotypic consequence in the organism. In some embodiments, the functional protein is associated with visible markers (including fluorescence), viability, fertility, fecundity, fitness, flight ability, vision, and behavioural differences. It will be appreciated, of course, that, in some embodiments, the expression systems are typically conditional, with the phenotype being expressed only under some, for instance restrictive, conditions.

The at least one heterologous polynucleotide sequence to be expressed in an organism is a heterologous sequence. By “heterologous,” it would be understood that this refers to a sequence that would not, in the wild type, be normally found in association with, or linked to, at least one element or component of the at least one splice control sequence. For example, where the splice control sequence is derived from a particular organism, and the heterologous polynucleotide is a coding sequence for a protein or polypeptide, i.e. is a polynucleotide sequence encoding a functional protein, then the coding sequence could be derived, in part or in whole, from a gene from the same organism, provided that the origin of at least some part of the transcribed polynucleotide sequence was not the same as the origin of the at least one splice control sequence. Alternatively, the coding sequence could be from a different organism and, in this context, could be thought of as “exogenous”. The heterologous polynucleotide could also be thought of as “recombinant,” in that the coding sequence for a protein or polypeptide are derived from different locations, either within the same genome (i.e., the genome of a single species or sub-species) or from different genomes (i.e., genomes from different species or subspecies), or synthetic sources.

Heterologous can refer to a sequence other than the splice control sequence and can, therefore, relate to the fact the promoter, and other sequences such as 5′UTR and/or 3′UTR can be heterologous to the polynucleotide sequence to be expressed in the organism, provided that said polynucleotide sequence is not found in association or operably linked to the promoter, 5′UTR and/or 3′UTR, in the wild type, i.e., the natural context of said polynucleotide sequence, if any.

It will be understood that heterologous also applies to “designer” or hybrid sequences that are not derived from a particular organism but are based on a number of components from different organisms, as this would also satisfy the requirement that the sequence and at least one component of the splice control sequence are not linked or found in association in the wild type, even if one part or element of the hybrid sequence is so found, as long as at least one part or element is not. It will also be understood that synthetic versions of naturally occurring sequences are envisioned. Such synthetic sequences are also considered as heterologous, unless they are of identical sequence to a sequence which would, in the wild type or natural context, be normally found in association with, or linked to, at least one element or component of the at least one splice control sequence.

This applies equally to where the heterologous polynucleotide is a polynucleotide for interference RNA.

In one embodiment, where the polynucleotide sequence to be expressed comprises a coding sequence for a protein or polypeptide, it will be understood that reference to expression in an organism refers to the provision of one or more transcribed RNA sequences, such as mature mRNAs, but this may also refer to translated polypeptides in said organism.

### Fusion Leaders

In some embodiments it will be desirable to have the functional protein of interest free of the Splice Control Module protein sequence. In some embodiments, the Splice Control Module is operatively linked to a polypeptide-encoding polynucleotide that stimulates proteolytic cleave of a translated polypeptide (“Fusion Leader Sequences” for the polynucleotide and “Fusion Leader Polypeptide” for the encoded polypeptide). An example of such a Fusion Leader Sequence is a ubiquitin-encoding polynucleotide. Such a Fusion Leader Sequence may be operatively linked in frame to the 3′ end of the Splice Control Module and operatively linked in frame to the protein encoding gene of interest (i.e., from 5′ to 3′: Splice Control Module-Fusion Leader Sequence-Gene of interest). In such a case, the Splice Control Module/Fusion Leader Polypeptide is cleaved from the protein of interest by specific proteases in the cell. Aside from ubiquitin, any other similar fusion may be made in place of ubiquitin that would have the effect of stimulating a cleavage of the N-terminal Splice Control Module. The ubiquitin fusion leader may be any polynucleotide encoding a functional ubiquitin leader polypeptide from any organism, provided that the ubiquitin leader is faithfully cleaved in the arthropod system. An example would be a Drosophila melanogaster ubiquitin that is cleaved from the functional protein that causes the lethal effect.

### Promoters and 5′UTRs

Each lethal gene is operably linked to a promoter, wherein said promoter is capable of being activated by an activating transcription factor or trans-activating encoded by a gene also included in at least one of the gene expression systems. Any combination of promoter and Splice Control Module is envisaged. In some embodiments, the promoter is specific to a particular protein having a short temporal or confined spatial effect, for example a cell-autonomous effect.

The promoter may be a large or complex promoter, but these often suffer the disadvantage of being poorly or patchily utilised when introduced into non-host insects. Accordingly, in some embodiments, it is preferred to employ minimal promoters. It will be appreciated that minimal promoters may be obtained directly from known sources of promoters, or derived from larger naturally-occurring, or otherwise known, promoters. Suitable minimal promoters and how to obtain them will be readily apparent to those skilled in the art. For example, suitable minimal promoters include a minimal promoter derived from Hsp70, a P minimal promoter, a CMV minimal promoter, an Act5C-based minimal promoter, a BmA3 promoter fragment, and an Adh core promoter (Bieschke, E. et al. (1998)Mol. Gen. Genet., 258:571-579). It is readily apparent to those skilled in the art as to how to ensure that the promoter selected is active. In some embodiments, at least one of the operably-linked promoters present in the invention is active during early development of the host organism, and preferably during embryonic stages, in order to ensure that the lethal gene is expressed during early development of the organism.

In some embodiments, the promoter can be activated by environmental conditions, for instance the presence or absence of a particular factor such as tetracycline (or analogue thereof) in the tet system described herein, such that the expression of the gene of interest can be easily manipulated by the skilled person.

Alternatively, the promoter may be specific for a broader class of proteins or a specific protein that has a long-term and/or wide system effect, such as a hormone, positive or negative growth factor, morphogen or other secreted or cell-surface signaling molecule. This would allow, for instance, a broader expression pattern so that a combination of a morphogen promoter with a stage-specific alternative splicing mechanism could result in the morphogen being expressed only once a certain life-cycle stage was reached, but the effect of the morphogen would still be felt (i.e., the morphogen can still act and have an effect) beyond that life-cycle stage. Examples include but are not limited to the morphogen/signaling molecules Hedgehog, Wingless/WNTs, TGFβ/BMPs, EGF and their homologues, which are well-known evolutionarily-conserved signaling molecules.

It is also envisaged that a promoter that is activated by a range of protein factors, for instance transactivators, or which has a broad systemic effect, such as a hormone or morphogen, could be used in combination with an alternative splicing mechanism to achieve a tissue and sex-specific control or sex and stage-specific control, or other combinations of stage-, tissue, germline- and sex-specific control.

It is also envisaged that more than one promoter, and optionally an enhancer therefor, can be used in the present system, either as alternative means for initiating transcription of the same protein or by virtue of the fact that the genetic system comprises more than one gene expression system (i.e., more than one gene and its accompanying promoter).

In some embodiments, at least one of the promoters is the minimal promoter is a heat shock promoter, such as Hsp70. Examples of sequences comprising Hsp70 promoters (HSP70 minipro) are SEQ ID NO:14, SEQ ID NO:40, and SEQ ID NO:67. In other embodiments, at least one of the promoters is the sry-α embryo-specific promoter (Horn and Wimmer (2003) Nat. Biotechnol. 21(1):64-70) from Drosophila melanogaster, or its homologues (an example is provided as SEQ ID NO:23), or promoters from other embryo-specific or embryo-active genes, such as that of the Drosophila gene slow as molasses (slam), or its homologues from other species.

In some embodiments, at least one of the promoters is a minimal promoter. In some embodiments, each of the promoters is independently Baculovirus Autographica californica nucleopolyhedrosisvirus (AcNPV) promoter IE1 (e.g., SEQ ID NO:30), Hsp70 (SEQ ID NO:14, SEQ ID NO:40, and SEQ ID NO:67), Hsp83 (SEQ ID NO:36), sry-α (SEQ ID NO:23) β-tubulin promoter (SEQ ID NO:38; SEQ ID NO:65), Protamine (SEQ ID NO:59; SEQ ID NO:85) and Mexfly actin promoter (SEQ ID NO:51 or SEQ ID NO:77 (with 5′UTR)) or Act5C or 3×P3. In some embodiments, one of the first and second promoters is Hsp70 and the other is IE1. In some embodiments, one of the first and second promoters is Hsp70 and the other is srya. In one embodiment, the first promoter is Hsp70 and the second promoter is srya. Each gene expression system further comprises a gene encoding an activating transcription factor, wherein each activating transcription factor activates the expression of a lethal gene of the transgene. Thus, each gene encoding an activating transcription factor is able to be expressed by the host organism, to produce a functional protein. In particular, each activating transcription factor is capable of activating at least one promoter, wherein the promoter is operably linked to a lethal gene. Consequently, when an activating transcription factor activates a promoter, the expression of the lethal gene operably linked to the promoter is up-regulated. Each activating transcription factor may act on either the first or the second promoter, or each activating transcription factor may act on both the first and the second promoter. In some embodiments, when more than one activating transcription factor is expressed, more than one promoter is activated. Thus, when both the first and the second activating transcription factors are expressed, both the first and the second promoters are activated. The gene products serving as activating transcription factors may act in any suitable manner. For example, the activating transcription factors may bind to an enhancer located in proximity to the at least one promoter, thereby serving to enhance polymerase binding at the promoter. Other mechanisms may be employed, such as repressor countering mechanisms, such as the blocking of an inhibitor of transcription or translation. Transcription inhibitors may be blocked, for example, by the use of hairpin RNA's or ribozymes to block translation of the mRNA encoding the inhibitor, or the product may bind the inhibitor directly, thereby preventing inhibition of transcription or translation.

### Repressible Elements

In some embodiments, the polynucleotide expression system is a recombinant dominant lethal genetic system, the lethal effect of which is conditional. Suitable conditions include temperature, so that the system is expressed at one temperature but not, or to a lesser degree, at another temperature, for example. The lethal genetic system may act on specific cells or tissues or impose its effect on the whole organism. It will be understood that all such systems and consequences are encompassed by the term lethal as used herein. Similarly, “killing”, and similar terms refer to the effective expression of the lethal system and thereby the imposition of a deleterious or sex-distorting phenotype, for example death.

In some embodiments, the polynucleotide expression system is a recombinant dominant lethal genetic system, the lethal effect of which is conditional and is not expressed under permissive conditions requiring the presence of a substance which is absent from the natural environment of the organism, such that the lethal effect of the lethal system occurs in the natural environment of the organism.

In some embodiments, the coding sequences encode a lethal effector protein linked to a system such as the tet system described in WO 01/39599 and/or WO 2005/012534.

In some embodiments, the expression of said lethal gene is under the control of a repressible transactivator protein. In certain embodiments, that the gene whose expression is regulated by alternative splicing encode a transactivator protein such as tTA, or variant thereof such as tTAV2 or tTAV3. Non-limiting examples of polynucleotides encoding tTAV proteins and variants include SEQ ID NO:34 and SEQ ID NO:61 (tTAV); SEQ ID NO:86 (tTAV2) and SEQ ID NO:87 (tTAV3). Proteins encoded by these are provided as SEQ ID NO:88 (tTAV), SEQ ID NO:89 (tTAV2) and SEQ ID NO:90 (tTAV3). This is not incompatible with the regulated protein being a lethal. In certain embodiments, it is both. In this regard, certain embodiments include a positive feedback mechanism as taught in WO2005/012534.

In some embodiments, the lethal effect of the dominant lethal system is conditionally repressible. In some embodiments, the lethal effect is exerted only in females. In other embodiments, the lethal effect is exerted only in males; that is, the lethal effect is expressed in males or females (as needed). For example, if the dominant lethal system is present in an insect, it is preferred that it leads to the death of at least 40% of the insects. In some embodiments, it leads to the death of at least 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% of the insects inheriting the system in the absence of the repressor.

Thus, in some embodiments wherein one or more of the dominant, lethal genes is tTA or a tTAV gene variant, an enhancer is a tetO element, comprising one or more tetO operator units. Upstream of a promoter, in either orientation, tetO is capable of enhancing levels of transcription from a promoter in close proximity thereto, when bound by the product of the tTA gene or a tTAV gene variant. In some embodiments, each enhancer is independently one of tetOx1, tetOx2, tetOx3, tetOx4, tetOx5, tetOx6, tetOx7, tetOx8, tetOx9, tetOx10, tetOx11, tetOx12, tetOx13, tetOx14, tetOx15, tetOx16, tetOx17, tetOx18, tetOx19, tetOx20 and tetOx21. In some embodiments, each enhancer is independently one of tetOx1, tetOx14 and tetOx21. In embodiments comprising more than one enhancer, the first enhancer is the same as or different from the second enhancer. An example of the tetOx7 element is shown in SEQ ID NO:15. An example of the tetOX14 is shown in SEQ ID NO:24. An example of tetOx21 element is shown in SEQ ID NO:39 or SEQ ID NO:66.

### Other Elements

In some embodiments, the system comprises other upstream, 5′ factors and/or downstream 3′ factors for controlling expression. Examples include enhancers such as the fat-body enhancers from the Drosophila yolk protein genes, and the homology region (hr) enhancers from baculoviruses, for example AcNPV Hr5. It will also be appreciated that the RNA products will include suitable 5′ and 3′ UTRs, for instance. Examples of 3′UTRs include, but are not limited to Drosophila melanogaster fs(1)K10 3′UTR (SEQ ID NO:5); SV40 3′UTR (SEQ ID NO:16; HSP83 3′UTR (SEQ ID NO:33 or SEQ ID NO:60); Protamine-like 3′UTR (SEQ ID NO:48); Mexfly Actin 3′UTR (SEQ ID NO:49 or SEQ ID NO:75); and Protamine 3′UTR (SEQ ID NO:78).

It will be understood that reference is made to start and stop codons between which the polynucleotide sequence to be expressed in an organism is defined, but that this does not exclude positioning of the at least one splice control sequence, elements thereof, or other sequences, such as introns, in this region. In fact, it will be apparent form the present description that the splice control sequence, can, in some embodiments, be positioned in this region.

Furthermore, the splice control sequence, for instance, can overlap with the start codon at least, in the sense that the G of the ATG can be, in some embodiments, be the initial 5′ G of the splice control sequence. Thus, the term “between” can be thought of as referring to from the beginning (3′ to the initial nucleotide, i.e., A) of the start codon, preferably 3′ to the second nucleotide of the start codon (i.e., T), up to the 5′ side of the first nucleotide of the stop codon. Alternatively, as will be apparent by a simple reading of a polynucleotide sequence, the stop codon may also be included.

### Fluorescence Expression

The invention also provides a method of quality control, for instance, by including a reporter such as a fluorescent protein, such as, for example, Green Fluorescent Protein (GFP) or any of the other colored fluorescent proteins known in the art. This may be the effector proteinper se, acting as a transformation marker. Other examples of fluorescent proteins used as transformation markers include ZsGreen, DsRed, DsRed2 and AmCyan.

Separate transformation markers may also be used, including those described here. Transcription of these transformation markers may be under the control of a separate promoter to that of the first or second expression unit. Examples of such promoters include muscle actin promoter, 3×P3, hrIE and hr5IE1.

In some embodiments, the fluorescent protein can be linked to the effector protein in the present system, so that this reporter protein and expression thereof will allow one to assess the degree of inclusion of a transgene or other effector into the population. This has at least some of the following advantages: first, as with any such marker it identifies the presence of the transgene, so one can follow inheritance. The more tightly the marker is linked to the trait of interest e.g. the lethal system, the less likely it is that mutations occur which will inactivate one but not the other. In practice, though, if they (the marker and transgene) are on the same inserted DNA segment this is extremely unlikely in any case. Second, if linked in the sense of fused, a marker shows expression of the effector protein. This would allow one to look at actual expression. For example, in certain embodiments of tet-repressible expression of a nuclease, fusion of the nuclease to a fluorescent reporter would allow one to check that insects to be released were expressing the nuclease. The presence of a fluorescent marker indicates (i) the male has at least one copy of the transgene; (ii) that the expression system is functioning correctly in that it expresses the effector in the absence of the repressor; (iii) that the insects are expressing the nuclease-FP fusion (and therefore were not, for example, inadvertently reared in the presence of the repressor); (iv) assuming male-specific expression, are male; (v) by inference they are indeed sterile. In quality control (QC) terms, the expression of a fluorescent protein gives much more certainty over whether the males are sterile than merely knowing that the male possesses a copy of the transgene. Third, with a higher-powered microscope one can see when the expression begins and where the protein is localized within the cell. This is a helpful development tool and also provides ease for monitoring consistency, such as in ongoing QC to establish whether the system remains consistent as in past performance, but also in the context of sperm-to-sperm consistency of expression; and (4) a further advantage is in respect of fluorescent sperm, discussed below.

There is a clear functional connection for a nuclease to cleave DNA, so if it is not in the nucleus it is unlikely to have the desired DNA-cleaving effect. Protamine acts as a nuclear localization signal (NLS) sequence, but in some embodiments, a nuclear localisation signal is provided to ensure that the nuclease is localized to the nucleus.

In a further aspect, a method of quality control is hereby provided, comprising inducing or de-repressing expression of the present expression system in a target group of individuals and determining whether those individuals meet expected criteria such as size, number, developmental stage or localization. For instance, if the system includes means to express a reporter such as a fluorescent protein, either as the effector or as part of a fusion protein for instance, then the individuals where expression from the system has been induced or de-repressed will become visible under suitable wavelengths of light.

Some embodiments of the present system include at least one spacer. Such spacers can advantageously be positioned between any of the present elements of the system. For instance, a spacer may be provided between the promoter and the regulatory elements and/or between the regulatory elements and the coding sequence, to thereby provide a “buffer” between these elements to ensure proper functionality thereof. As such, the spacer has no function in gene expression other than to separate these elements although it may optionally include a number of restriction sites, if this is deemed to be useful. Ideally, it should not include any transcription binding factor sites, etc. as these might interfere with expression of the effector.

In some embodiments, the effector may be in the form of a fusion sequence or protein, such that, for instance, a nuclease is fused to a marker such that transcription and translation of the effector also leads to transcription and translation of the marker. This has the advantage of showing exposure of a sperm to a nuclease, the presence of the fluorescent protein being indicative that the nuclease has been expressed. The fluorescent proteins may be viewed under fluorescence microscopy using excitation filters suitable for the particular fluorescent protein.

It is also envisaged that the present system and methods can be used to produce fluorescent sperm. For instance, a reporter such as those mentioned above could be linked to the promoter or, indeed, under a separate promoter, such as tetO promoter enhancer system if the effector is tTA or any of its variants. Fluorescent sperm would be advantageous for visual separation of sperm or gonads, particularly in methods of dissection or sex selection. In particular, it infers the ability to determine with which male individual a female has mated, which is useful in the context of a field release program. Such a method might include, providing (e.g. trapping) wild females; dissecting them; looking for stored sperm and see whether such sperm carry the present system, i.e. are fluorescent. This will very quickly demonstrate whether a female:
(i) is unmated (has not yet mated);
(ii) mated with a wild type male (as it shows non-fluorescent sperm);
(iii) mated with a transgenic male carrying the present system (which would show fluorescent sperm; or
(iv) mated both types of male (shown by the presence of fluorescent and nonfluorescent sperm).
   Introduction of Constructs into Organisms

Methods of introduction or transformation of the gene system constructs and induction of expression are well known in the art with respect to the relevant organism. It will be appreciated that the system or construct may be administered as a plasmid, but generally tested after integrating into the genome. Plasmid vectors may be introduced into the desired host cells by methods known in the art, such as, for example by transfection, electroporation, microinjection, transduction, cell fusion, DEAE dextran, calcium phosphate precipitation, lipofection (lysosome fusion), use of a gene gun, or a DNA vector transporter (see, e.g., Wu et al., (1992) J. Biol. Chem. 267:963; Wu et al. (1988) J. Biol. Chem. 263:14621; and Canadian Patent Application No. 2,012,311 to Hartmut et al.). Administration by microinjection into embryos is the typical method of creating genetically engineered arthropods (e.g. insects). The plasmid may be linearised before or during administration. The plasmid vector may be integrated into the host chromosome by any means known. Well-known methods of locus-specific insertion may be used, including homologous recombination and recombinase-mediated genome insertion. In another embodiment, locusspecific insertion may be carried out by recombinase-site specific gene insertion. In one example piggyBac sequences may be incorporated into the vector to drive insertion of the vector into the host cell chromosome. Other technologies such as CRISPRs, TALENs, AttP/AttB recombination may also be employed. Not all of the plasmid may be integrated into the genome. Where only part of the plasmid is integrated into the genome, it is preferred that this part include the at least one splice control module capable of mediating alternative splicing, and the cassette to provide male sterility.

The expression units of the gene expression system may be introduced on the same or separate constructs. For example, the expression unit that imparts female lethality may be introduced on a separate construct than the expression unit that imparts male sterility. One of skill in the art would readily be able to fashion various constructs to incorporate the expression units into the target organism in order to achieve an integrated, functional gene expression system comprising the expression units described herein.

### Genetically Engineered Insects

The vectors of the invention may be used to create transgenic tephritid fruit flies such as Medfly (Ceratitis capitata), Mexfly (Anastrepha ludens), Oriental fruit fly (Bactrocera dorsalis), Spotted-wing drosophila (Drosophila suzukii), Olive fruit fly (Bactrocera oleae), Melon fly (Bactrocera cucurbitae), Natal fruit fly (Ceratitis rosa), Cherry fruit fly (Rhagoletis cerasi), Queensland fruit fly (Bactrocera tyroni), Peach fruit fly (Bactrocera zonata) Caribbean fruit fly (Anastrepha suspensa) or West Indian fruit fly (Anastrepha obliqua).

### Specific Embodiments

In a specific embodiment, a Cctra splice control module is used for sex-specific expression in an insect. In this embodiment, the Cctra splice control module is derived from Ceratitis capitata and incorporates both introns and exons from the C. capitata transformer gene (Cctra). In some embodiments, the Cctra splice control module comprises, Exon 1a, Exon 1b, Intron 1, Exon 2a, Exon 2b, and Intron 2 of Cctra. In some embodiments, the Cctra splice control module has a polynucleotide sequence of SEQ ID NO:7, wherein the Introns and Exons have the following polynucleotide sequences: Exon 1a (SEQ ID NO:8); Exon 1b (SEQ ID NO:9); Intron 1 (SEQ ID NO:10); Exon 2a (SEQ ID NO:11); Exon 2b (SEQ ID NO:12); and Intron 2 (SEQ ID NO:13). The splice control module may also be a Bactrocera zonata tra splice control module (Bztra). The module may comprise, for example, Exon 1, Intron 1, Exon 2, and Intron 2. In some embodiments, the Bztra splice control module has a polynucleotide sequence of SEQ ID NO:18, wherein the Introns and Exons have the following polynucleotide sequences: Exon 1 (SEQ ID NO:19); Intron 1 (SEQ ID NO:20); Exon 2 (SEQ ID NO:21); and Intron 2 (SEQ ID NO:22).

In some embodiments, the constructs and transformed arthropods such as insects (e.g., Ceratitis capitata) contain more than one splicing module operably connected to a gene of interest such as a gene imparting a lethal effect. In some embodiments, the splicing modules are the same type of splicing module, such as 2 or more Cctra splicing modules or two or more Bztra splicing modules. In other embodiments, there may be different types of splicing modules such as at least one Cctra and at least one Bztra splicing module.

In some embodiments, portions of the Introns and Exons are used (preserving the splice donor and splice acceptor sites of each) but that are truncated to reduce the size of the overall splice control module. Alterations of the introns and exons may be made, provided that splice donor and acceptor sites are preserved and that exons are spliced for sex-specific expression and open reading frames are preserved for sex-specific expression of the gene of interest (e.g., a gene to impart lethality to one of the sexes). Variations of the splice control modules maybe found, for example in US 2009/0183269A1.

In certain embodiments, the sex-specific alternative splicing is a construct with elements from OX3864A, which is more fully described in US 2009/0183269A1. These elements may be incorporated on the same construct or a separate construct from the elements that impart male sterility (discussed in further detail below in the Examples).

The insects of the specific embodiment also contain a cassette for testes-specific expression of a gene that is damaging to sperm cells to render the males sterile. In some embodiments, the cassettes comprise a promoter (such as a D. melanogaster Hsp70 mini promoter) operatively linked to a splicing cassette to direct testes-specific expression of a gene of interest. The splicing cassette may be for example a C. capitata Protamine or Protamine B cassette comprising a Protamine or Protamine B 5′UTR and Protamine or Protamine B introns and exons to allow for testis-specific expression of a gene that is damaging to sperm cells which is operatively linked 3′ of the cassette. In some embodiments, the cassette comprises a Protamine cassette with the sequence of SEQ ID NO:68. In other embodiments, the cassette comprises a Protamine B cassette with the sequence of SEQ ID NO:41. In the Protamine splicing module of SEQ ID NO:68, the module comprises a C. capitata Protamine 5′UTR/CDS of SEQ ID NO:69, a C. capitate Protamine Exon 1 of SEQ ID NO:70, a C. capitata Protamine Intron of SEQ ID NO:71, and a C. capitata Protamine Exon 2 of SEQ ID NO:72. In the Protamine B splicing module of SEQ ID NO:41, the module comprises a D. melanogaster Protamine B 5′UTR of SEQ ID NO:42, a D. melanogaster Protamine B Exon 1 of SEQ ID NO:46, a D. melanogaster Protamine B Intron 1 of SEQ ID NO:43, a D. melanogaster Protamine B Exon 2 of SEQ ID NO:44, a D. melanogaster Protamine B Intron 2 of SEQ ID NO:91, and a D. melanogaster Protamine B Exon 3 of SEQ ID NO:92.

### Methods of Suppressing Populations of Arthropods/Insects and Reducing Crop Damage

The invention also provides methods of suppressing populations of wild-type arthropods, such as tephritid fruit flies, by releasing genetically engineered male arthropods (e.g., tephritid fruit flies) comprising an expression system of the invention, among a population of wildtype male insects of the same species, whereupon the genetically engineered insects mate with the wild-type insects and the offspring of such matings do not hatch as the parental males produce non-functional sperm and are therefore sterile, so none of the parental female's eggs are fertilized.

Insects may be reared for breeding by including a compound to repress expression of the functional protein and rescuing the insects from the lethal effect such that more adult insects may be produced. The male insects will also not express the nuclease which renders them sterile. Thus, in the presence of the repressing compound or condition, both male and female insects survive and are fertile. In the absence of the repressing compound or condition, the protein having a lethal effect is expressed in both males and females, such that females die before reaching adulthood, leaving only males, which due to the expression of the sterilizing gene, are rendered sterile.

The invention also provides methods reducing, inhibiting or eliminating crop damage from arthropods (such as tephritid fruit flies) comprising releasing genetically engineered male arthropods (e.g., tephritid fruit flies) comprising an expression system of the invention, among a population of wild insects of the same species, whereupon the genetically engineered insects mate with the wild insects and the offspring of such matings do not hatch as the parental males produce non-functional sperm and are therefore sterile, so none of the parental females' eggs are fertilized with viable sperm, thereby suppressing the population of wild insects and reducing, inhibiting or eliminating crop damage caused by the wild insects.

### EXAMPLES

### Example 1. Construction of Medfly Cassettes for Male Sterility

A. Ceratitis capitata Strains and Constructs for Inhibiting Sperm Development

The following experiments utilized the TOLIMAN (wildtype (WT)) strain of Ceratitis capitata. This strain was originally collected in Guatemala and transferred to Oxitec from the Food and Agriculture Organization/International Atomic Energy Agency (FAO/IAEA) El Piño mass rearing facility in 2004, and has been maintained for approximately 210 generations. All strains were reared under standard insectary conditions: 25° C. (±2° C.), 55% (±10%) relative humidity (RH), 12 h: 12 h light: dark cycle. Eggs were collected and resulting larvae allowed to develop on larval diet. Adults were fed with a ratio of 1:4 volumes yeast powder and sugar. For on-tet reared groups, the larval rearing medium contained 100 μg/ml tetracycline hydrochloride; adults were supplied ultrapure (MilliQ) water with equivalent tetracycline.

### (i) Microinjection and Strain Development

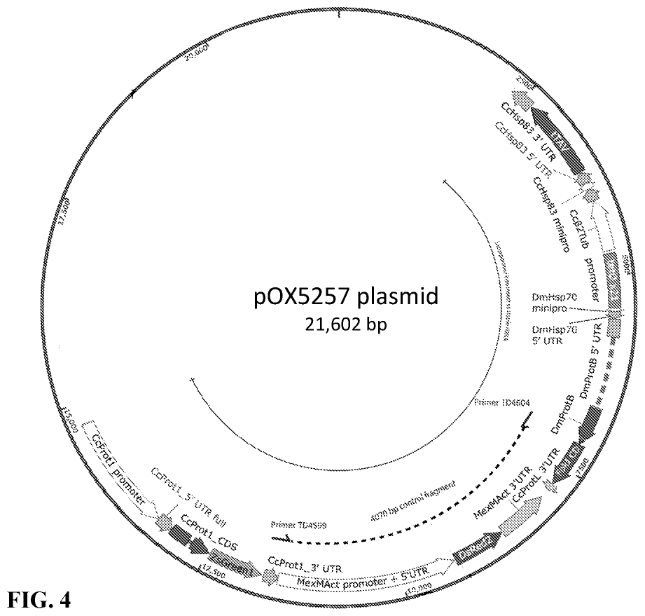

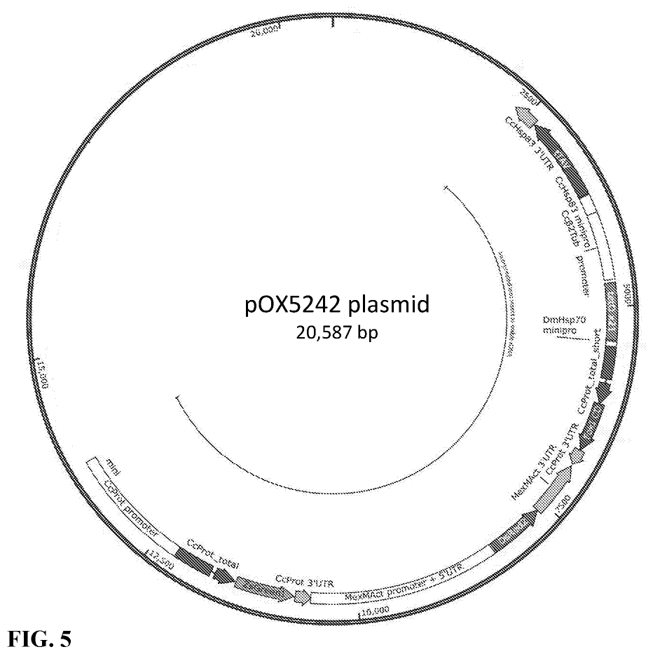

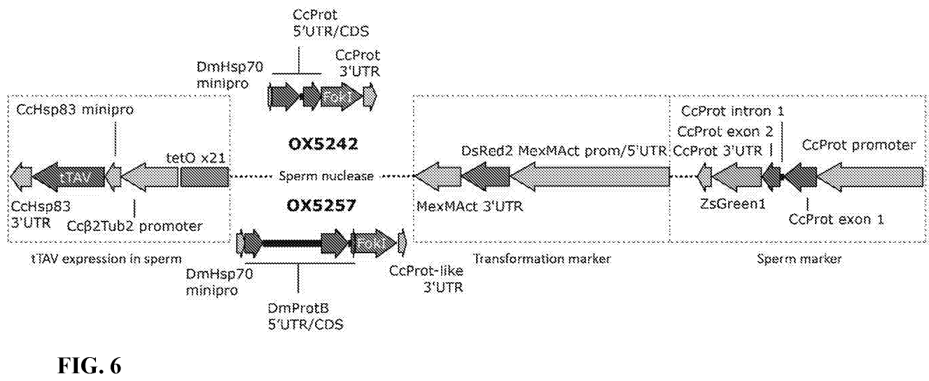

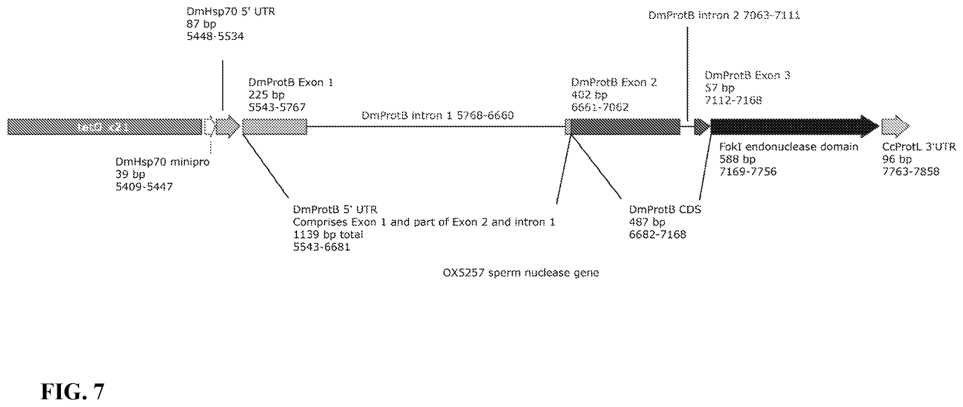

C. capitata WT eggs were transformed by microinjection. Either pOX5257 (FIG. 4) or pOX5242 plasmid DNA (FIG. 5) (600 ng/μl) with piggyBac helper plasmid pOX3022 (300 ng/l), which when translated is the source of transposase, were injected into eggs. Table 1A shows the genetic elements present on the pOX5257 plasmid, while Table 1B shows the genetic elements present on the pOX5242 plasmid. The piggyBac DNA construct (either of pOX5242 or pOX5257) and the transposase pDNA (pOX3022) were reconstituted in injection buffer (5 mM KCl, 0.1 mM NaH2PO4, pH 6.8) made with standard laboratory grade reagents, to which 100 μg/ml tetracycline was added (Handler et al. (1998) Proc. Natl. Acad. Sci. USA 95:7520-7525). The OX5242 and OX5257 constructs were injected into 3310 and 6315 pre-blastoderm WT embryos, respectively. Adult survivors that had been injected with pOX5242 or pOX5257, at the preblastodermal egg stage (G0) were screened for fluorescence and 826 OX5242 and 607 OX5257 G0 adults were back-crossed with WT, in small pools (5-15 G0 males were crossed with 15-30 WT females and 10-30 G0 females were crossed with 10 WT males). G1 pupae were screened for fluorescence (MexMAct-DsRed2 fluorescent marker). Fluorescent scoring of G1 progeny identified 7 and 3 transgenic strains for OX5242 and OX5257, respectively. Detailed maps of the pOX5257 and pOX5242 are shown in FIG. 6 and FIG. 7.

Table 1A. Genetic elements of OX5257 incorporated into the medfly genome
Table 1B. Genetic elements of OX5242 incorporated into the medfly genome

### (ii) Penetrance and Repressibility

Viable eggs were produced from all transgenic strains (OX5242H, OX5242(2)H1, OX5242G, OX5242P, OX5242Y, OX5242AC, OX5242AL; OX5257B, OX5257V, and OX5257AX). All strains were assessed for the penetrance and repressibility of male sterility. A single transgenic G1 male or female from a given pool was backcrossed with WT (2 males or 3 females). Thereafter, strains were maintained by backcrossing hemizygous male individuals with WT females in a 1:2 ratio.

To assess penetrance and repressibility of the early bisex self-limiting trait in OX5242 or OX5257, eggs from the WT and hemizygous early bisex research colonies (ten strains of OX5242 or OX5257) were independently collected off-tet and on-tet. Eclosing males (n=5) taken from each experimental group, were crossed independently with off-tet reared WT females (n=10), in cages (10 cm×10 cm×10 cm). Eggs were collected in 24-hour intervals (n=100 eggs) three times, from each group (days 5, 6 and 7 post-eclosion). The eggs were maintained in a humid chamber (an inverted Petri dish sealed with Parafilm) and the hatch rate was assessed after five days. Six WT (off-tet) and six WT (on-tet) controls had been performed, as the strains were generated across a period of months. The mean hatch rates from all six observations were used as the off-tet and on-tet control values. Penetrance and repressibility were calculated based on mean hatch rates of progeny from the hemizygous early bisex strains (reared off-tet and on-tet), relative to the equivalently reared WT controls. The results are shown in Table 2.

TABLE 2

| | Mean % Hatch Rate (±SE) | | Penetrance | | | Repressibility | | |
|---|---|---|---|---|---|---|---|---|
| Line | Off-tet | On-tet | % | X² | P_{(df)} | % | X² | P_{(df)} |
| OX5242P | 0 | 78.0 ± 2.4 | 100 | 514.6 | <0.001_{[1]} | 87 | 16.1 | <0.001_{[1]} |
| OX5257V | 0 | 67.3 ± 2.7 | 100 | 514.6 | <0.001_{[1]} | 75 | 45.9 | <0.001_{[1]} |
| OX5242AL | 0 | 53.0 ± 2.9 | 100 | 514.6 | <0.001_{[1]} | 59 | 100.8 | <0.001_{[1]} |
| OX5242H | 1.3 ± 0.7 | 73.7 ± 2.5 | 99 | 498.9 | <0.001_{[1]} | 82 | 26.9 | <0.001_{[1]} |
| OX5257AX | 2.0 ± 0.8 | 59.3 ± 2.8 | 98 | 491.2 | <0.001_{[1]} | 66 | 74.6 | <0.001_{[1]} |
| OX5257B | 3.0 ± 1.0 | 69.0 ± 2.7 | 97 | 479.9 | <0.001_{[1]} | 77 | 40.6 | <0.001_{[1]} |
| OX5242AC | 4.7 ± 1.2 | 67.0 ± 2.7 | 95 | 461.5 | <0.001_{[1]} | 75 | 47.0 | <0.001_{[1]} |
| OX5242G | 86.0 ± 2.0 | 84.3 ± 2.1 | 7 | 6.2 | 0.01_{[1]} | | | |
| OX5242(2)H1 | 86.7 ± 2.0 | 85.7 ± 2.0 | 6 | 5.1 | 0.02_{[1]} | | | |
| OX5242Y | 93.3 ± 1.4 | 90.3 ± 1.7 | 0 | 0.23 | 0.63_{[1]} | | | |
| WT | 92.4 ± 1.5 | 89.4 ± 1.7 | | | | | | |

¹OX5242(2)H denotes that the strain was produced in round 2 of microinjection. Underlining indicates inadequate penetrance or repressibility.

Three lines (OX5242P, OX5257V and OX5242AL) appeared to demonstrate fully penetrant male sterility (>99.90%) in the hemizygous state; hatching was not observed (n=300 eggs). A further four lines were >9500 penetrant (OX5242H, OX5242AC, OX5257B and OX5257AX). Three lines (OX5242Y, OX5242G and OX5242(2)H) were insufficiently penetrant (<95%). The repressibility of the seven penetrant lines varied widely (61-87%; mean: 74%). Of these, two lines (OX5242AX and OX5242AL) were substantially less repressible than the mean (A=130%), and hence discarded.

(iii) Performance of Fluorescent Sperm Marking

The visibility of the fluorescent sperm marker (Ccprot1-ZsGreen) in testes of hemizygous males, and in the reproductive tract of females to which these males had mated, were next assessed. Crosses identical to those described above were initiated. Mating was allowed for 7-9 days. Individuals were removed from the cage and from males and females respectively, the testes and spermathecae were dissected in “testis buffer” (187 mM KCl, 47 mM NaCl, 10 mM Tris pH 6.8) and imaged (Motic BA210 microscope, Fraen fluorescence FLUOLED lamp, and Lumenera Infinity 2 camera).

Off-tet reared hemizygous males of four of five strains (all except OX5242P), demonstrated reliable transfer of fluorescently labelled sperm to females. However, the number of sperm transferred was reduced, relative to WT controls. Furthermore, sperm were frequently immotile and/or demonstrated morphological differences (lack of curliness). For on-tet reared males, the quantity and morphology of sperm transferred to females, were similar to controls (summarised in Table 3).

TABLE 3

| | Visibility of fluorescent sperm | |
|---|---|---|
| | marking in the female reproductive tract, post-mating | |
| | Females mated to off-tet | Females mated to on-tet |
| | reared males | reared males |
| OX5242H | Sperm marked in 10/11 | Sperm marked in 11/11 |
| | females, generally | females, generally |
| | 500-1000 present. | >1500 present |
| OX5257V | Sperm marked in 9/9 | Sperm marked in 10/10 |
| | females, generally 100 or | females, generally |
| | fewer sperm present | about 1000 present |
| OX5257B | Sperm marked in 9/11 | Sperm marked in 11/11 |
| | females, generally | females, generally |
| | 100-1000 present | >1500 present |
| OX5242AC | Sperm marked in 9/11 | Sperm marked in 11/11 |
| | females, generally | females, generally |
| | 100 sperm present | 500-1500 present |
| OX5242P | Sperm marked in only 1/13 | Sperm marked in 12/12 |
| | females, generally fewer | females, generally |
| | than 50 sperm present | >1500 present |

| | |
|---|---|
| WT | >1500 sperm generally present, with no |
| | obvious difference between off-tet and |
| | on-tet reared samples (>100 females dissected) |

Consistent with these findings, sperm were deficient in testes of off-tet reared hemizygous males. The localisation of the fluorescent sperm marker was also diffused in sperm. For males reared on-tet, these effects were mostly reversed. Mature spermatozoa were frequently observed, and the localisation of the fluorescent sperm marker was sharper and brighter, relative to off-tet reared males. Off-tet and on-tet reared WT controls were indistinguishable and demonstrated normal development. Therefore, the observed effects result from sperm nuclease expression, rather than an independent effect of tetracycline. From these results, OX5242P, OX5242AC, OX5257B, OX5242H, and OX5257V were selected for further studies.

### (iv) Insertion Site and Homozygous Viability

Hemizygous males of the five strains (OX5242H, OX5242P, OX5242AC, OX5257B & OX5257V) were backcrossed with WT females. The inheritance pattern of the fluorescent marker linked to OX5242 or OX5257 (MexMAct-DsRed2), indicated that all five strains carried single insertions, without significant fitness penalty in the hemizygous state (Table 4). Furthermore, the observation of male and female progeny that carried the fluorescent marker, from all crosses, indicated that the strains were all autosomal insertions (Y-linked insertions would yield male-only fluorescent progeny; X-linked insertions would yield female-only fluorescent progeny for males crossed with wild-type females). These assumptions were confirmed by Southern blot in a parallel analysis (not shown).

TABLE 4

| | Fluorescent progeny | | | | Fluorescent males and females |
|---|---|---|---|---|---|
| Strain | % | n | χ2 | P_{[df]} | in ratio of 1:1 observed |
| OX5242H | 46.8 | 675 | 1.4 | 0.24_{[1]} | Yes |
| OX5257B | 47.8 | 408 | 0.4 | 0.53_{[1]} | Yes |
| OX5242AC | 48.9 | 219 | 0.1 | 0.81_{[1]} | Yes |
| OX5242P | 48.9 | 374 | 0.1 | 0.77_{[1]} | Yes |
| OX5257V | 53.0 | 332 | 0.1 | 0.4_{[1]} | Yes |

“n”: number of individuals.
χ2: chi-square value.
P: significance value.
_{[df]}degrees of freedom.

Finally, hemizygous males of the five strains were independently crossed with hemizygous females of the same strain. The inheritance ratio of the MexMAct-DsRed2 fluorescent marker in the progeny, indicated that all five strains were homozygous viable (Table 5). The inheritance ratio in the progeny of all strains did not significantly differ from the expected value for a homozygous viable line without fitness penalties (75%), except for OX5257V. The expected value for a completely inviable line is 66.6%. The inheritance ratio in OX5257V (70%) was significantly different to the expected value (75%) for a fully viable strain (n=332, χ2=4.1, p=0.04, df=1), indicating a potential fitness penalty of homozygosity.

TABLE 5

| | Fluorescent progeny | | | |
|---|---|---|---|---|
| Strain | % | n | χ2 | P[df] |
| OX5242H | 78.5 | 256 | 0.9 | 0.35[1] |
| OX5242AC | 74.1 | 559 | 0.1 | 0.72[1] |
| OX5242P | 72.7 | 918 | 1.3 | 0.25[1] |
| OX5257V | 70.0 | 654 | 4.1 | 0.04[1] |

“n”: number of individuals.
χ2: chi-square value.
P: significance value.
[df]: degrees of freedom.

(v) Summary of Selection of Ceratitis capitata Strains

Strains were selected as candidates to introgress with OX3864A into stacked trait product candidate strains, based on: penetrance and repressibility of the early bisex phenotype; ability of males to transfer fluorescently labelled sperm to females; indication of a single, autosomal insertion that is homozygous viable; and the ability to remove piggyflac ends. Strains that met these criteria included OX5242P, OX5242H, OX5242AC, OX5257V, and OX5257B. A summary is shown in Table 6.

TABLE 6

| | Selection Criteria | | | | | |
|---|---|---|---|---|---|---|
| | | | | | Homo- | |
| | Pene- | Repressi- | Sperm | Insertion | zygous | piggyBac |
| Strain | trance | bility | transfer | site | viability | removal |
| OX5242H | ✓ | ✓ | ✓ | ✓ | ✓ | ✓ |
| OX5257V | ✓ | ✓ | ✓ | ✓ | ✓ | ✓ |
| OX5242AC | ✓ | ✓ | ✓ | ✓ | ✓ | ✓ |
| OX5242P | ✓ | ✓ | x | ✓ | ✓ | ✓ |
| OX5242AL | ✓ | x | x | x | x | x |
| OX5257AX | ✓ | x | x | x | x | x |
| OX5242G | x | x | x | x | x | x |
| OX5242Y | x | x | x | x | x | x |
| OX5242(2)H | x | x | x | x | x | x |

### Example 2: Generation of Double Homozygous SLI Sterile Male Medflies

A. OX3864A Strain and Sex-Specific Survival Off-tet

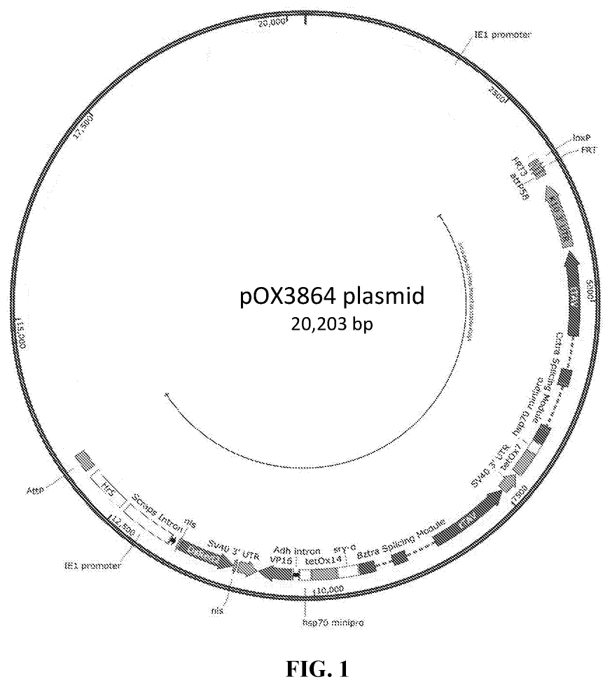

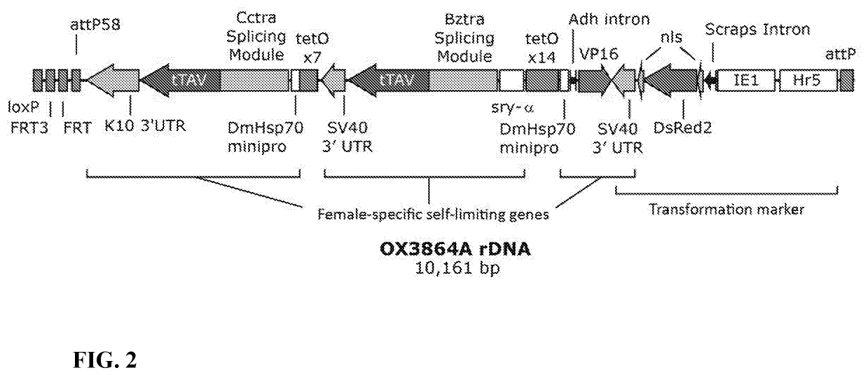

To obtain a C. capitata strain with two stacked self-limiting traits, male selection and male sterility, male selection was bred into the OX5242H and OX5257V strains. OX3864A, a conditional female-specific self-limiting strain of C. capitata shown diagrammatically in FIG. 1, was independently crossed with OX5242H and OX5257V, which contain an early bisex construct to provide male sterility shown diagrammatically in FIG. 2.

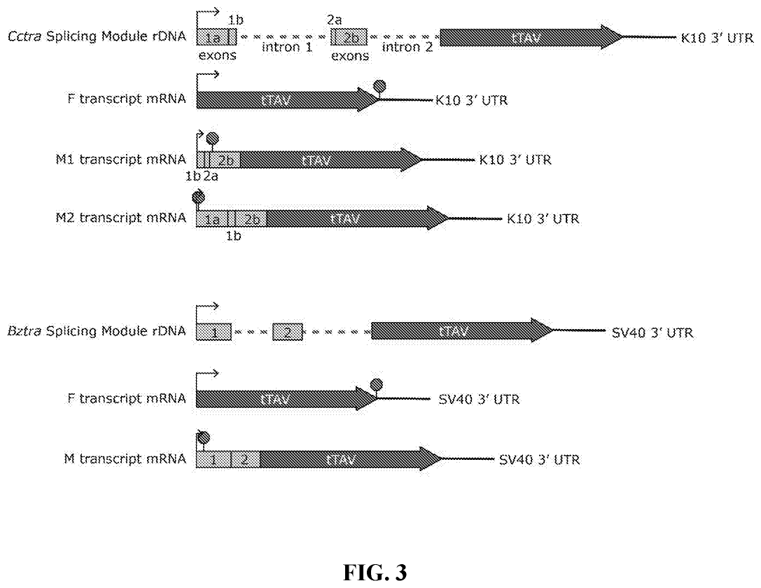

OX3864A construction was shown previously (WO 2015/185933). The OX3864A construct contains two splicing modules to provide sex-specific splicing of a tra-tTAVfusion such that the transcripts are spliced by females to obtain a functional tTAV protein whereas in males, the male splicing pattern results in a truncated, incomplete tTAV that is non-functional. The elements of OX3864A are shown in Table 7. The splicing pattern of the OX3864A splicing modules is shown diagrammatically in FIG. 3. In one case, the splicing module is a C. capitata-specific tra (Cctra) splicing module operably linked to a tTAV transcript. In another, it is a Bactrocera zonata-specific tra (Bztra) operably linked to a tTAVtranscript.

Table 7. Elements of OX3864A Bred into OX5257 C. capitata Strains

### B. Crosses and Generation of Stacked Trait Double Homozygous Flies

The piggyBac ends were first removed in the OX5242H and OX5257V strains, and confirmed absent by standard molecular methods (endpoint PCR and Sanger sequencing) (see Dafa'alla, T. H. et al. (2006) Nat. Biotechnol. 24(7):820-821). OX5242H-hemizygous or OX5257V-hemizygous individuals were crossed with OX3864A-homozygous individuals. F1 double hemizygous males and females were selected by fluorescence microscopy, and crossed with one another. F2 double homozygous individuals were selected by fluorescence microscopy and thereafter verified by PCR to confirm that all individuals were double-homozygous. Double homozygous, stacked trait colonies were generated for OX5242H-OX3864A (n=16 individuals) and OX5257V-OX3864A (n=14 individuals).

### Sequence Listing Free Text

### SEQ ID NO: 1: Synthetic DNA

### SEQ ID NO: 2 Synthetic DNA

### SEQ ID NO: 3: Synthetic DNA

### SEQ ID NO: 4: Synthetic DNA

SEQ ID NO: 6: Synthetic DNA encoding fusion of tet activator protein optimized for insect expression
SEQ ID NO: 15: Synthetic DNA contains 7 repeats of the Tn10 tet-operon
SEQ ID NO: 16: Synthetic DNA non-coding fragment based on Simian virus (SV40)
SEQ ID NO: 17: Synthetic DNA encoding the fusion tetracycline transactivator protein optimized for expression in insects
SEQ ID NO: 24: Synthetic DNA contains 14 repeats of the Tn10 tet-operon
SEQ ID NO: 26: Synthetic DNA used as a transcriptional activator

### SEQ ID NO: 27: Synthetic DNA

SEQ ID NO: 28: Synthetic DNA derived from Dicosoma (Clontech)

### SEQ ID NO: 32: Synthetic DNA

SEQ ID NO: 34: Synthetic DNA based on a fusion of sequences from E. coli (tetR-tetracycline repressor) and HSV-1 (VP16 transcriptional activator)
SEQ ID NO: 39: Synthetic DNA contains 21 repeats of the Tn10 tet-operon
SEQ ID NO: 47: Codon optimized FokI from Flavobacterium okeanokoites
SEQ ID NO: 50: Synthetic DNA encoding variant of red protein from Discocoma (Clontech)
SEQ ID NO: 53: Synthetic DNA encoding variant of green protein from Zoanthus (Clontech)
SEQ ID NO: 61: Synthetic DNA based on a fusion of sequences from E. coli (tetR-tetracycline repressor) and HSV-1 (VP16 transcriptional activator)
SEQ ID NO: 66: Synthetic DNA contains 21 repeats of the Tn10 tet-operon
SEQ ID NO: 73: Codon optimized FokI from Flavobacterium okeanokoites
SEQ ID NO: 76: Synthetic DNA derived from Dicosoma (Clontech)
SEQ ID NO: 79: Synthetic DNA encoding variant of green protein from Zoanthus (Clontech)
SEQ ID NO: 86: Synthetic DNA based on a fusion of sequences from E. coli (tetR-tetracycline repressor) and HSV-1 (VP16 transcriptional activator)
SEQ ID NO: 87: Synthetic DNA based on a fusion of sequences from E. coli (tetR-tetracycline repressor) and HSV-1 (VP16 transcriptional activator)
SEQ ID NO: 88: Synthetic protein based on a fusion of sequences from E. coli (tetR-tetracycline repressor) and HSV-1 (VP16 transcriptional activator)
SEQ ID NO: 89: Synthetic protein based on a fusion of sequences from E. coli (tetR-tetracycline repressor) and HSV-1 (VP16 transcriptional activator)
SEQ ID NO: 90: Synthetic protein based on a fusion of sequences from E. coli (tetR-tetracycline repressor) and HSV-1 (VP16 transcriptional activator)
SEQ ID NO: 94: Expression vector unit for integration
SEQ ID NO: 95: Expression vector unit for integration
SEQ ID NO: 96: A variant of red fluorescent protein originally identified in Discosoma (Clontech)
SEQ ID NO: 97: Exons 2 and 3 of Ceratitis capitata Protamine fused to FokI coding region
SEQ ID NO: 98: Fusion of Exons 2 and 3 of Ceratitis capitata Protamine with FokI
SEQ ID NO: 99: Exons 2 and 3 of Drosophila melanogaster Protamine B fused to FokI coding region
SEQ ID NO: 100: Fusion of Exons 2 and 3 of D. melanogaster ProtamineB with FokI
SEQ ID NO: 102: Fusion of sequences from E. coli (tetR—tetracycline repressor) and HSV-1 (VP16 transcriptional activator)

1-100. (canceled)

101. A gene expression system for controlled expression of an effector gene in an arthropod comprising:
(a) a first expression unit comprising:
i. a first promoter that functions in an arthropod operably linked to a 5′UTR/CDS gene sequence;
ii. an effector gene operably linked to said 5′UTR/CDS;
iii. a 3′UTR operably linked to said effector gene; and
iv. a repressible element operably linked to said promoter, wherein transcription of said effector gene is repressible;
(b) a second expression unit comprising a coding sequence for a transcription factor operably linked to an upstream regulatory element, said transcription factor capable of acting upon said first promoter of said first expression unit to drive expression of a said effector gene, wherein said upstream regulatory element comprises:
i. a first promoter/5′UTR comprising a gene promoter operably linked to a corresponding d gene 5′UTR;
ii. a second promoter/5′UTR operably linked to said first promoter/5′UTR wherein said second promoter/5′UTR is adjacent to a start site for the transcription of said transcription factor coding sequence;
wherein said first promoter/5′UTR and said second promoter/5′UTRare capable of being preferentially expressed in the arthropod testes, when used together; and wherein said upstream regulatory element drives sufficient expression of said transcription factor such that said transcription factor drives transcription of said effector gene and
(c) at least one third expression unit comprising:
i. a polynucleotide encoding a functional protein, the coding sequence of which is defined between a start codon and a stop codon;
ii. a second promoter capable of initiating transcription in said arthropod operably linked to said polynucleotide; and
iii. a splice control polynucleotide which, in cooperation with a spliceosome in said arthropod, is capable of sex-specifically mediating in said arthropod
(A) a first splicing of an RNA transcript of said polynucleotide to produce a first spliced mRNA product, which does not have a continuous open reading frame extending from said start codon to said stop codon; and
(B) an alternative splicing of said RNA transcript to yield an alternatively spliced mRNA product which comprises a continuous open reading frame extending from said start codon to said stop codon, wherein said functional protein has a lethal effect on said arthropod
wherein said third expression unit is repressible.

102. The gene expression system of claim 101, wherein the system is an inducible system, where induction or repression occurs by provision or absence of a chemical entity.

103. The gene expression system of claim 102, wherein said chemical entity is tetracycline or an analogue thereof.

104. The gene expression system of claim 101, wherein said first promoter is a minimal promoter selected from an HSP70 minipro promoter, a mini p35 promoter, a mini CMV promoter (CMVm), an Ac5 promoter, a polyhedron promoter, or a UAS promoter.

105. The gene expression system of claim 101, wherein said 5′UTR/CDS gene sequence is a protamine 5′UTR/CDS or Protamine B gene sequence.

106. The gene expression system of claim 101, wherein said 3′UTR is testes-specific or from the same gene as said 5′UTR/CDS gene sequence; and/or said 3′UTR is a protamine or protamine-like 3′UTR.

107. The gene expression system of claim 101, wherein the effector gene encodes a nuclease or an interfering RNA.

108. The gene expression system of claim 107, wherein said nuclease is a 3-Zn finger nuclease.

109. The gene expression system of claim 108, wherein said 3-Zn finger nuclease is a FokI nuclease.

110. The gene expression system of claim 101, wherein said first promoter/5′UTR comprises a topi, aly or β-tubulin promoter or homologue thereof, operably linked to a corresponding topi, aly or β-tubulin 5′UTR.

111. The gene expression system of claim 101 wherein said transcription factor in said second expression unit is tTA or a variant thereof selected from tTAV, tTAV2, or tTAV3.

112. The gene expression system of claim 101, wherein said transcription factor of said second expression unit is tTA or a variant thereof, and the first expression unit comprises a tet operator (tetO).

113. The gene expression system of claim 101, wherein said functional protein is an apoptosis-inducing factor, Hid, Reaper (Rpr), or NipplDm.

114. The gene expression system of claim 101, wherein said RNA transcript comprises two or more coding exons for said functional protein.

115. The gene expression system of claim 101, wherein said third expression unit comprises at least one positive feedback mechanism, having at least one functional protein to be differentially expressed, via alternative splicing, and at least one promoter therefor, wherein a product of a gene to be expressed serves as a positive transcriptional control factor for the at least one promoter therefor, and whereby the expression of said product is suppressible.

116. The gene expression system of claim 101, wherein an enhancer is associated with said second promoter, and wherein said functional protein enhances activity of said second promoter via said enhancer.

117. The gene expression system of claim 101, wherein splice control is determined by a tTA gene product or an analogue thereof, and wherein one or more tetO operator units is operably linked with the promoter and is the enhancer, tTA or its analogue serving to enhance activity of the promoter via tetO.

118. The gene expression system of claim 101, wherein the functional protein itself a transcriptional transactivator, such as the tTAV system, comprising tTAV, tTAV2 or tTAV3.

119. The gene expression system of claim 101, wherein said third expression unit is activated by the presence or absence of a chemical entity.

120. The gene expression system of claim 119, wherein said chemical entity is tetracycline or an analogue thereof.

121. The gene expression system of claim 101, wherein said second promoter is a srya embryo-specific promoter, or a homologue thereof, an Hsp70 promoter, or homologue thereof, or a Drosophila slow as molasses (slam) promoter or a homologue thereof.

122. The gene expression system of claim 101, wherein said splice control polynucleotide is derived from a tra gene selected from the Ceratitis capitata transformer gene (Cctra), the Drosophila transformer gene (Dmtra), the Ceratitis rosa transformer gene (Crtra), or the Bactrocera zonata transformer gene (Bztra), or from at least a fragment of a doublesex (dsx) gene, such as that derived from a Drosophila spp., Ceratitis spp., Bombyx mori, Pink Boll Worm, Codling Moth, or a mosquito, in particular Aedes gambiae or Aedes aegypti.

123. The gene expression system of claim 101, wherein said splice control polynucleotide comprises an intron and wherein said intron comprises on its 5′ end, a guanine (G) nucleotide, in RNA, or wherein said splice control polynucleotide comprises an intron and wherein said intron comprises on its 5′ end, UG nucleotides, and UT at its 3′ end, in RNA.

124. The gene expression system of claim 101, wherein said arthropod is an insect.

125. An arthropod comprising the arthropod gene expression system of claim 101.

126. The arthropod of claim 125 wherein said arthropod is an insect.

127. The arthropod of claim 126 wherein said insect is a Ceratitis capitata or Ceratitis rosa.

128. A method of suppressing a wild population of an arthropod comprising breeding a stock of male arthropods comprising the gene expression system of claim 101 and distributing said stock of male arthropods at a locus of a population of wild arthropods of the same species to be suppressed, whereby matings between said stock male arthropods and said wild arthropods are non-productive due to a detrimental effect on the sperm cells of said male arthropods, thereby suppressing said wild population.

129. The method according to claim 128, wherein the detrimental effect on said sperm cells of said male arthropods is conditional and occurs by expression of said effector gene, the expression of said effector gene being under the control of a repressible transactivator protein, the said breeding being under permissive conditions in the presence of achemical ligand, the chemical ligand being absent from the said natural environment and able to repress said transactivator.

130. The method of claim 129 wherein said chemical ligand is tetracycline or an analogue thereof.

131. A method of rearing sterile male arthropods comprising rearing a stock of male and female arthropods transformed with the gene expression system of claim 101 under conditions that activates transcription of the gene expression system, allowing survival of male, but not female arthropods.

132. The method of claim 131, wherein said arthropod is an insect selected from a Medfly (Ceratitis capitata), a Mexfly (Anastrepha ludens), an Oriental fruit fly (Bactrocera dorsalis), a Spotted-wing drosophila (Drosophila suzukii), an Olive fruit fly (Bactrocera oleae), a Melon fly (Bactrocera cucurbitae), a Natal fruit fly (Ceratitis rosa), a Cherry fruit fly (Rhagoletis cerasi), a Queensland fruit fly (Bactrocera tyroni), a Peach fruit fly (Bactrocera zonata), a Caribbean fruit fly (Anastrepha suspensa) or a West Indian fruit fly (Anastrepha obliqua).
